SEQUENCE LISTING (78 sequences)

SEQ ID NO: 1 - protein, 242 aa, Homo sapiens
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 2 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 3 - protein, 122 aa, Artificial Sequence
  Synthetic Construct
EVQLVESGGGLVQPGGSLRLSCAASGFTFSSTWIHWVRQAPGKGLEWVARISPYYYYSDYADSVKGRFTISADTSKNTAYLQMNSLRAEDTAVYYCARGLGKGSKRGAMDYWGQGTLVTVSS

SEQ ID NO: 4 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQSFYFPNTFGQGTKVEIKR

SEQ ID NO: 5 - protein, 122 aa, Artificial Sequence
  Synthetic Construct
EVQLVESGGGLVQPGGSLRLSCAASGFTFSSTWIHWVRQAPGKGLEWVARISPYSGYTNYADSVKGRFTISADTSKNTAYLQMNSLRAEDTAVYYCARGLGKGSKRGAMDYWGQGTLVTVSS

SEQ ID NO: 6 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 7 - protein, 122 aa, Artificial Sequence; identical to SEQ ID NO: 5
  Synthetic Construct

SEQ ID NO: 8 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYTALPYTFGQGTKVEIKR

SEQ ID NO: 9 - protein, 122 aa, Artificial Sequence; identical to SEQ ID NO: 5
  Synthetic Construct

SEQ ID NO: 10 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYSDVPYTFGQGTKVEIKR

SEQ ID NO: 11 - protein, 122 aa, Artificial Sequence; identical to SEQ ID NO: 5
  Synthetic Construct

SEQ ID NO: 12 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYSDLPYTFGQGTKVEIKR

SEQ ID NO: 13 - protein, 122 aa, Artificial Sequence
  Synthetic Construct
EVQLVESGGGLVQPGGSLRLSCAASGFTFSSTWIHWVRQAPGKGLEWVARISPYSGYTNYADSVKGRFTISADTSKNTAYLQMNSLRAEDTAVYYCARGINQGSKRGAMDYWGQGTLVTVSS

SEQ ID NO: 14 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQSRTARPTFGQGTKVEIKR

SEQ ID NO: 15 - protein, 118 aa, Artificial Sequence
  Synthetic Construct
EVQLVESGGGLVQPGGSLRLSCAASGFTFTSNYIHWVRQAPGKGLEWVASITPASGDTDYADSVKGRFTISADTSKNTAYLQMNSLRAEDTAVYYCARSRGAYRFAYWGQGTLVTVSS

SEQ ID NO: 16 - protein, 108 aa, Artificial Sequence; identical to SEQ ID NO: 6
  Synthetic Construct

SEQ ID NO: 17 - protein, 25 aa, Artificial Sequence [sequence omitted]

SEQ ID NO: 18 - protein, 13 aa, Artificial Sequence
  Synthetic Construct
WVRQAPGKGLEWV

SEQ ID NO: 19 - protein, 32 aa, Artificial Sequence
  Synthetic Construct
RFTISADTSKNTAYLQMNSLRAEDTAVYYCAR

SEQ ID NO: 20 - protein, 11 aa, Artificial Sequence
  Synthetic Construct
WGQGTLVTVSS

SEQ ID NO: 21 - protein, 23 aa, Artificial Sequence
  Synthetic Construct
DIQMTQSPSSLSASVGDRVTITC

SEQ ID NO: 22 - protein, 15 aa, Artificial Sequence
  Synthetic Construct
WYQQKPGKAPKLLIY

SEQ ID NO: 23 - protein, 32 aa, Artificial Sequence
  Synthetic Construct
GVPSRFSGSGSGTDFTLTISSLQPEDFATYYC

SEQ ID NO: 24 - protein, 12 aa, Artificial Sequence
  Synthetic Construct
TFGQGTKVEIKR

SEQ ID NO: 25 - protein, 118 aa, Artificial Sequence
  Synthetic Construct
EVQLVESGGGLVQPGGSLRLSCAASGFSFTSYGISWVRQAPGKGLEWVSHIDWYGGDTDYADSVKGRFTISADTSKNTAYLQMNSLRAEDTAVYYCARGGPDYAMDYWGQGTLVTVSS

SEQ ID NO: 26 - protein, 108 aa, Artificial Sequence
  Synthetic Construct
DIQMTQSPSSLSASVGDRVTITCRASQSISSYLAWYQQKPGKAPKLLIYGASSRASGVPSRFSGSGSGTDFTLTISSLQPEDFATYYCQQYWSEPVTFGQGTKVEIKR

SEQ ID NO: 27 - protein, 14 aa, Artificial Sequence
  Synthetic Construct
ARGINQGSKRGAMD

SEQ ID NO: 28 - protein, 14 aa, Artificial Sequence
  Synthetic Construct
ARGLGKGSKRGAMD

SEQ ID NO: 29 - protein, 10 aa, Artificial Sequence
  Synthetic Construct
ARSRGAYRFA

SEQ ID NO: 30 - protein, 7 aa, Artificial Sequence
  Synthetic Construct
GFTFSST

SEQ ID NO: 31 - protein, 7 aa, Artificial Sequence
  Synthetic Construct
GFTFTSN

SEQ ID NO: 32 - protein, 13 aa, Artificial Sequence
  Synthetic Construct
GINQGSKRGAMDY

SEQ ID NO: 33 - protein, 13 aa, Artificial Sequence
  Synthetic Construct
GLGKGSKRGAMDY

SEQ ID NO: 34 - protein, 10 aa, Artificial Sequence
  Synthetic Construct
LLIYSASFLY

SEQ ID NO: 35 - protein, 5 aa, Artificial Sequence
  Synthetic Construct
PASGD

SEQ ID NO: 36 - protein, 5 aa, Artificial Sequence
  Synthetic Construct
PYSGY

SEQ ID NO: 37 - protein, 5 aa, Artificial Sequence
  Synthetic Construct
PYYYY

SEQ ID NO: 38 - protein, 8 aa, Artificial Sequence
  Synthetic Construct
QQSFYFPN

SEQ ID NO: 39 - protein, 9 aa, Artificial Sequence
  Synthetic Construct
QQSFYFPNT

SEQ ID NO: 40 - protein, 8 aa, Artificial Sequence
  Synthetic Construct
QQSRTARP

SEQ ID NO: 41 - protein, 9 aa, Artificial Sequence
  Synthetic Construct
QQSRTARPT

SEQ ID NO: 42 - protein, 8 aa, Artificial Sequence
  Synthetic Construct
QQSYTTPP

SEQ ID NO: 43 - protein, 9 aa, Artificial Sequence
  Synthetic Construct
QQSYTTPPT

SEQ ID NO: 44 - protein, 8 aa, Artificial Sequence
  Synthetic Construct
QQYSDLPY

SEQ ID NO: 45 - protein, 9 aa, Artificial Sequence
  Synthetic Construct
QQYSDLPYT

SEQ ID NO: 46 - protein, 8 aa, Artificial Sequence
  Synthetic Construct
QQYSDVPY

SEQ ID NO: 47 - protein, 9 aa, Artificial Sequence
  Synthetic Construct
QQYSDVPYT

SEQ ID NO: 48 - protein, 8 aa, Artificial Sequence
  Synthetic Construct
QQYTALPY

SEQ ID NO: 49 - protein, 9 aa, Artificial Sequence
  Synthetic Construct
QQYTALPYT

SEQ ID NO: 50 - protein, 11 aa, Artificial Sequence
  Synthetic Construct
RASQDVSTAVA

SEQ ID NO: 51 - protein, 17 aa, Artificial Sequence
  Synthetic Construct
RISPYSGYTNYADSVKG

SEQ ID NO: 52 - protein, 17 aa, Artificial Sequence
  Synthetic Construct
RISPYYYYSDYADSVKG

SEQ ID NO: 53 - protein, 7 aa, Artificial Sequence
  Synthetic Construct
SASFLYS

SEQ ID NO: 54 - protein, 17 aa, Artificial Sequence
  Synthetic Construct
SITPASGDTDYADSVKG

SEQ ID NO: 55 - protein, 5 aa, Artificial Sequence
  Synthetic Construct
SNYIH

SEQ ID NO: 56 - protein, 9 aa, Artificial Sequence
  Synthetic Construct
SRGAYRFAY

SEQ ID NO: 57 - protein, 6 aa, Artificial Sequence
  Synthetic Construct
SSTWIH

SEQ ID NO: 58 - protein, 7 aa, Artificial Sequence
  Synthetic Construct
STAVAWY

SEQ ID NO: 59 - protein, 5 aa, Artificial Sequence
  Synthetic Construct
STWIH

SEQ ID NO: 60 - protein, 6 aa, Artificial Sequence
  Synthetic Construct
TSNYIH

SEQ ID NO: 61 - protein, 13 aa, Artificial Sequence
  Synthetic Construct
WVARISPYSGYTN

SEQ ID NO: 62 - protein, 13 aa, Artificial Sequence
  Synthetic Construct
WVARISPYYYYSD

SEQ ID NO: 63 - protein, 13 aa, Artificial Sequence
  Synthetic Construct
WVASITPASGDTD

SEQ ID NO: 64 - protein, 5 aa, Artificial Sequence
  Synthetic Construct
  VARIANT 2: Xaa = Asn or Thr
  VARIANT 3: Xaa = Tyr or Trp
SXXIH

SEQ ID NO: 65 - protein, 17 aa, Artificial Sequence
  Synthetic Construct
  VARIANT 1: Xaa = Ser or Arg
  VARIANT 3: Xaa = Thr or Ser
  VARIANT 5: Xaa = Ala or Tyr
  VARIANT 6: Xaa = Ser or Tyr
  VARIANT 7: Xaa = Gly or Tyr
  VARIANT 8: Xaa = Asp or Tyr
  VARIANT 9: Xaa = Thr or Ser
  VARIANT 10: Xaa = Asp or Asn
XIXPXXXXXXYADSVKG

SEQ ID NO: 66 - protein, 13 aa, Artificial Sequence
  Synthetic Construct
  VARIANT 2: Xaa = Leu or Ile
  VARIANT 3: Xaa = Gly or Asn
  VARIANT 4: Xaa = Lys or Gln
GXXXGSKRGAMDY

SEQ ID NO: 67 - protein, 11 aa, Artificial Sequence
  Synthetic Construct
  VARIANT 3: Xaa = Ser or Tyr
  VARIANT 5: Xaa = Tyr, Thr, Ser, Phe, or Arg
  VARIANT 7: Xaa = Thr, Ala, Asp, or Tyr
  VARIANT 8: Xaa = Thr, Leu, Val, Phe, or Ala
  VARIANT 9: Xaa = Pro or Arg
  VARIANT 10: Xaa = Pro, Tyr, or Asn
QQXIXPXXXXT

SEQ ID NO: 68 - protein, 108 aa, Artificial Sequence
  Synthetic Construct
EVQLVESGGGLVQPGGSLRLSCAASGFTFSSNWIHWVRQAPGKGLEWVARISPNSGYTDYADSVKGRFTISADTSKNTAYLQMNSLRAEDTAVYYCARSMRARRGFDY

SEQ ID NO: 69 - protein, 108 aa, Artificial Sequence; identical to SEQ ID NO: 6
  Synthetic Construct

SEQ ID NO: 70 - protein, 5 aa, Artificial Sequence
  Synthetic Construct
SNWIH

SEQ ID NO: 71 - protein, 17 aa, Artificial Sequence
  Synthetic Construct
RISPNSGYTDYADSVKG

SEQ ID NO: 72 - protein, 10 aa, Artificial Sequence
  Synthetic Construct
SMRARRGFDY

SEQ ID NO: 73 - protein, 5 aa, Artificial Sequence
  Synthetic Construct
SYGIS

SEQ ID NO: 74 - protein, 17 aa, Artificial Sequence
  Synthetic Construct
HIDWYGGDTDYADSVKG

SEQ ID NO: 75 - protein, 9 aa, Artificial Sequence
  Synthetic Construct
GGPDYAMDY

SEQ ID NO: 76 - protein, 11 aa, Artificial Sequence
  Synthetic Construct
RASQSISSYLA

SEQ ID NO: 77 - protein, 7 aa, Artificial Sequence
  Synthetic Construct
GASSRAS

SEQ ID NO: 78 - protein, 9 aa, Artificial Sequence
  Synthetic Construct
QQYWSEPVT

(19) United States
(12) Patent Application Publication
(10) Pub. No.: US 2018/0222974 A1
(43) Pub. Date: Aug. 9, 2018

(54) COMPOSITIONS AND METHODS FOR USING ANTI-IL-34 ANTIBODIES TO TREAT NEUROLOGICAL DISEASES

(71) Applicant: Genentech, Inc., South San Francisco, CA (US)

(72) Inventors: Courtney EASLEY-NEAL, San Mateo, CA (US); Robby WEIMER, Half Moon Bay, CA (US); Ali Akbar ZARRIN, Brisbane, CA (US)

(73) Assignee: Genentech, Inc., South San Francisco, CA (US)

(21) Appl. No.: 15/826,512

(22) Filed: Nov. 29, 2017

Related U.S. Application Data
(63) Continuation of application No. PCT/US2016/035342, filed on Jun. 1, 2016.
(60) Provisional application No. 62/335,028, filed on May 11, 2016.
(60) Provisional application No. 62/170,069, filed on Jun. 2, 2015.

(51) Int. Cl. C07K 16/24; A61P 25/28; C07K 16/46; A61K 39/395; A61K 38/17; C07K 16/28; A61P 25/14; A61P 25/16; A61P 25/00

(52) U.S. Cl. CPC C07K 16/244; A61K 2039/507; C07K 16/468; C07K 16/243; A61K 39/3955; A61K 38/1709; C07K 16/2866; A61P 25/14; A61P 25/16; A61P 25/00; C07K 2317/32; C07K 2317/76; C07K 2317/56; C07K 2317/31; A61P 25/28

(57) ABSTRACT

The invention provides methods for treating neurological diseases and reducing microglia density using anti-IL-34 antibodies.

### CROSS-REFERENCE TO RELATED APPLICATIONS

This application is a continuation of International Application No. PCT/US2016/035342 filed internationally on Jun. 1, 2016, which claims the benefit of U.S. Provisional Application No. 62/170,069, filed Jun. 2, 2015, and U.S. Provisional Application No. 62/335,028, filed May 11, 2016, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392031701SEQLIST.txt, date recorded: Nov. 29, 2017, size: 42 KB).

### FIELD OF THE INVENTION

The present invention relates to compositions and methods for using anti-IL-34 antibodies to treat neurological diseases.

### BACKGROUND

Neurological diseases, including neurodegenerative diseases, impact hundreds of millions of people worldwide. Neurological diseases are disorders of the central and peripheral nervous system, and involve the brain, spinal cord, cranial nerves, peripheral nerves, nerve roots, autonomic nervous system, neuromuscular junction, and muscles (WHO, February 2014). Neurodegenerative diseases, such as Alzheimer's disease, Parkinson's disease, and Huntington's disease, are characterized by the death or malfunction of nervous system cells, leading to symptoms such as cognitive and motor deficits.

Alzheimer's disease, the leading cause of dementia, is ranked as the sixth leading cause of death in the United States (National Center for Health Statistics, CDC, 2013). Over 5.4 million Americans are currently diagnosed with Alzheimer's disease, and over 500,000 people die each year due to the disease. Alzheimer's disease is accompanied by significant societal and healthcare costs. In 2013, it is estimated that 15.5 million caregivers provided $220 billion in unpaid care for Alzheimer's disease patients. In 2014, healthcare associated costs for Alzheimer's diseases were estimated to be $214 billion. Alzheimer's disease prevalence is expected to increase dramatically, with a predicated 16 million by 2050. (Alzheimer's Disease Foundation March 2015).

Alzheimer's disease is characterized by the accumulation of extracellular plaques, composed of beta-amyloid peptide, and neurofibrillary tangles, composed of the protein tau, in the brain. Subsequent death of neurons in the cerebral cortex and subcortical regions of the brain lead to neurodegeneration. Symptoms of Alzheimer's disease include memory loss, confusion, difficulty speaking, motor deficits, and changes in mood or personality. Parkinson's disease is a chronic, progressive neurodegenerative disease characterized by dementia and progressive motor dysfunction. Parkinson's disease is caused by the death of dopamine producing neurons in the central nervous system. In most people, Parkinson's disease is idiopathic (having no known cause). However, a small portion of cases have a genetic link. Huntington's disease is a neurodegenerative genetic disorder caused by an autosomal dominant mutation on either of the two copies of a gene located on chromosome 4, called huntingtin (htt). Expansion of a CAG (cytosine-adenine-guanine) triplet repeat stretch within the Huntingtin gene results in production of a modified form of the huntingtin protein, which progressively damages cells in the brain. As the disease progresses, symptoms include severe motor, cognitive, and psychiatric disturbances.

Neuroinflammation and microgliosis are believed to play a role in neurodegenerative diseases. Neuroinflammation is characterized by activation of central nervous system cells and production of inflammatory mediators. Microgliosis involves the abnormal proliferation or hypertrophy of microglia, resident central nervous system macrophages, in response to inflammatory signals. Neuroinflammation and microgliosis can promote the mechanisms underlying neurodegenerative diseases, such as plaque accumulation in Alzheimer's disease and neuronal death and dysfunction in Parkinson's disease and Huntington's disease (Block et al., (2005) Progress in Neurobiology 76 (2): 77-98; Moller (2010) J Neural Transm 117(8):1001-1008). Chronic neuroinflammation and microgliosis also occur in other neurodegenerative and neurodevelopmental diseases such as amyotrophic lateral sclerosis (ALS), prion disease, spinocerebellar ataxia, spinal muscular atrophy, autism, and autism spectrum disorders (Amor et al., (2014) Immunology 142(2):151-166; El-Ansary et al. (2012) J of Neuroinflammation 9:265).

There is currently no cure for Alzheimer's diseases or other neurological diseases. For example, current drugs for Alzheimer's disease focus on regulating neurotransmitters in order to treat symptoms of the disease, such as motor and cognitive deficits. However, these drugs show limited efficacy and do not halt disease progression.

Thus, an unmet need exists for novel therapeutic approaches for neurological diseases, in particular for approaches that target underlying disease pathology.

All references cited herein, including patent applications and publications, are hereby incorporated by reference in their entirety.

### SUMMARY

Described herein are methods of treating a neurological disease that meet the need for novel therapeutic approaches.

Thus one aspect includes methods of treating a neurological disease in an individual comprising administering to the individual an effective amount of an anti-IL-34 antibody. In some embodiments, the individual has the neurological disease or has been diagnosed with the neurological disease. In some embodiments, the density of microglia in the brain of the individual is reduced. In some embodiments, the density of dendritic spines near amyloid plaques in the brain of the individual is increased.

Another aspect includes methods of treating an individual exhibiting one or more symptoms of a neurological disease comprising administering to the individual an effective amount of an anti-IL-34 antibody. In some embodiments, the one or more symptoms are selected from a group consisting of memory loss, confusion, disorientation, mood changes, and behavior changes. In some embodiments, the one or more symptoms improve after administration of an effective amount of the anti-IL-34 antibody. In some embodiments, the one or more symptoms are measured using the Mini-Mental State Examination.

Yet another aspect includes methods of reducing the density of microglia in the brain of an individual comprising administering to the individual an effective amount of an anti-IL-34 antibody. In some embodiments, the density of microglia in the brain is reduced by at least 30%, by at least 40%, by at least 50%, by at least 60%, by at least 70%, or by at least 80%.

In some embodiments, the anti-IL-34 antibody is an isolated antibody that binds to human IL-34, which antibody binds to an epitope comprising at least one of amino acid residues Glu103, Leu109, Gln106, Asn150, Leu127, Asn128, Ser184, Leu186, Asn187, Lys44, Glu121, Asp107, Glu111, Ser104, Gln120, Trp116, and Asn61 of a human IL-34, wherein the position of the amino acid residues is based on the position in SEQ ID NO:1, and wherein the antibody inhibits the binding between human IL-34 and human CSF-1R.

In some embodiments, the anti-IL-34 antibody is an isolated antibody that binds to human IL-34, which antibody binds to an epitope comprising at least one of amino acid residues from Glu103 to Asn150 of a human IL-34, wherein the position of the amino acid residues is based on SEQ ID NO:1, and wherein the antibody inhibits the binding between human IL-34 and human CSF-1R.

In some embodiments, the antibody binds to an epitope comprising at least one of amino acid residues Glu103, Leu109, Gln106, and Asn150 of the human IL-34, wherein the position of the amino acid residues is based on the position in SEQ ID NO:1. In some embodiments, the epitope further comprises at least one of amino acid residues Ser100, Glu123, Trp116, Thr124, Leu127, Asn128, Gln131, and Thr134 of the human IL-34, wherein the position of the amino acid residues is based on the position in SEQ ID NO:1. In some embodiments, the antibody binds to amino acids within positions 100-108, 116-134, 109 and 150 of the human IL-34, and wherein the position of the amino acid residues is based on the position in SEQ ID NO:1.

In some embodiments, the antibody binds to an epitope comprising at least one of amino acid residues Asn128, Ser184, Leu186, Asn187, Lys44, and Glu121 of the human IL-34, wherein the position of the amino acid residues is based on the position in SEQ ID NO:1. In some embodiments, the epitope further comprises at least one of amino acid residues Phe40, Asp43, Leu125, Gln189, Thr36, and Val185 of the human IL-34, wherein the position of the amino acid residues is based on the position in SEQ ID NO:1. In some embodiments, the antibody binds to amino acids within positions 36-44, 121-128, and 184-187 of the human IL-34, and wherein the position of the amino acid residues is based on the position in SEQ ID NO:1.

In some embodiments, the antibody binds to an epitope comprising at least one of amino acid residues from Glu103-Leu127 of the human IL-34, wherein the position of the amino acid residues is based on the position in SEQ ID NO:1. In some embodiments, the antibody binds to an epitope comprising at least one of amino acid residues Asp107, Glu111, Ser104, Gln120, Glu103, Leu109, Trp116, and Asn61 of the human IL-34, wherein the position of the amino acid residues is based on the position in SEQ ID NO:1. In some embodiments, the epitope further comprises at least one of amino acid residues Pro152, Val108, Leu110, Gln106, Glu123, Leu127, Lys117, Ile60 and Lys55 of the human IL-34, wherein the position of the amino acid residues is based on the position in SEQ ID NO:1. In some embodiments, the antibody binds to amino acids within positions 55-61, 100-108, 109, 111-127 and 152 of the human IL-34, and wherein the position of the amino acid residues is based on the position in SEQ ID NO:1.

In some embodiments, the antibody comprises a heavy chain variable region sequence of at least 90% sequence identity to the amino acid sequence of SEQ ID NO:3 and/or a light chain variable region sequence of at least 90% sequence identity to the amino acid sequence of SEQ ID NO:4. In some embodiments, the antibody comprises a heavy chain variable region sequence of the amino acid sequence of SEQ ID NO:3 and/or a light chain variable region sequence of the amino acid sequence of SEQ ID NO:4. In some embodiments, the antibody comprises (a) a HVR-H3 comprising an amino acid sequence GLGKGSKRGAMDY (SEQ ID NO: 33); (b) a HVR-L3 comprising an amino acid sequence QQSFYFPNT (SEQ ID NO: 39); and (c) a HVR-H2 comprising an amino acid sequence RISPYYYYSDYADSVKG (SEQ ID NO: 52). In some embodiments, the antibody comprises (a) a HVR-H1 comprising an amino acid sequence STWIH (SEQ ID NO: 59); (b) a HVR-H2 comprising an amino acid sequence RISPYYYYSDYADSVKG (SEQ ID NO: 52); and (c) a HVR-H3 comprising an amino acid sequence GLGKGSKRGAMDY (SEQ ID NO: 33). In some embodiments, the antibody comprises (a) a HVR-L1 comprising an amino acid sequence RASQDVSTAVA (SEQ ID NO: 50); (b) a HVR-L2 comprising an amino acid sequence SASFLYS (SEQ ID NO: 53); and (c) a HVR-L3 comprising an amino acid sequence QQSFYFPNT (SEQ ID NO: 39).

In some embodiments, the antibody binds to a dimer of the IL-34. In some embodiments, the antibody binds to an epitope that spans over both protomers of the human IL-34 dimer. In some embodiments, the anti-IL-34 antibody is an isolated antibody that binds to a human IL-34, wherein the antibody inhibits the binding between human IL-34 and human CSF-1R, and wherein the antibody binds to a dimer of the IL-34.

In some embodiments, the antibody comprises (a) a HVR-H3 comprising an amino acid sequence GLGKGSKRGAMDY (SEQ ID NO: 33) or GINQGSKRGAMDY (SEQ ID NO: 32); (b) a HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41); and (c) a HVR-H2 comprising an amino acid sequence RISPYYYYSDYADSVKG (SEQ ID NO: 52) or RISPYSGYTNYADSVKG (SEQ ID NO: 51). In some embodiments, the antibody comprises (a) a HVR-H1 comprising an amino acid sequence STWIH (SEQ ID NO: 59); (b) a HVR-H2 comprising an amino acid sequence RISPYYYYSDYADSVKG (SEQ ID NO: 52) or RISPYSGYTNYADSVKG (SEQ ID NO: 51); and (c) a HVR-H3 comprising an amino acid sequence GLGKGSKRGAMDY (SEQ ID NO: 33) or GINQGSKRGAMDY (SEQ ID NO: 32). In some embodiments, the antibody comprises (a) a HVR-L1 comprising an amino acid sequence RASQDVSTAVA (SEQ ID NO: 50); (b) a HVR-L2 comprising an amino acid sequence SASFLYS (SEQ ID NO: 53); and (c) a HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41) or QQSFYFPN (SEQ ID NO: 38) or QQSYTTPP (SEQ ID NO: 42) or QQYTALPY (SEQ ID NO: 48) or QQYSDLPY (SEQ ID NO: 44) or QQYSDVPY (SEQ ID NO: 46) or QQSRTARP (SEQ ID NO: 40).

In some embodiments, the antibody comprises (a) a HVR-H3 comprising an amino acid sequence GLGKGSKRGAMDY (SEQ ID NO: 33); (b) a HVR-L3 comprising an amino acid sequence QQYSDLPYT (SEQ ID NO: 45); and (c) a HVR-H2 comprising an amino acid sequence RISPYSGYTNYADSVKG (SEQ ID NO: 51). In some embodiments, the antibody comprises (a) a HVR-H1 comprising an amino acid sequence of STWIH (SEQ ID NO: 59); (b) a HVR-H2 comprising an amino acid sequence RISPYSGYTNYADSVKG (SEQ ID NO: 51); and (c) a HVR-H3 comprising an amino acid sequence GLGKGSKRGAMDY (SEQ ID NO: 33). In some embodiments, the antibody comprises (a) a HVR-L1 comprising an amino acid sequence of RASQDVSTAVA (SEQ ID NO: 50); (b) a HVR-L2 comprising an amino acid sequence SASFLYS (SEQ ID NO: 53); and (c) a HVR-L3 comprising an amino acid sequence QQYSDLPYT (SEQ ID NO: 45).

In some embodiments, the antibody comprises a heavy chain variable region sequence of at least 90% sequence identity to the amino acid sequence of SEQ ID NO:5 and/or a light chain variable region sequence of at least 90% sequence identity to the amino acid sequence of SEQ ID NO:6. In some embodiments, the antibody comprises a heavy chain variable region sequence of the amino acid sequence of SEQ ID NO:5 and/or a light chain variable region sequence of the amino acid sequence of SEQ ID NO:6. In some embodiments, the antibody comprises a heavy chain variable region sequence of at least 90% sequence identity to the amino acid sequence of SEQ ID NO:7 and/or a light chain variable region sequence of at least 90% sequence identity to the amino acid sequence of SEQ ID NO:8. In some embodiments, the antibody comprises a heavy chain variable region sequence of the amino acid sequence of SEQ ID NO:7 and/or a light chain variable region sequence of the amino acid sequence of SEQ ID NO:8. In some embodiments, the antibody comprises a heavy chain variable region sequence of at least 90% sequence identity to the amino acid sequence of SEQ ID NO:9 and/or a light chain variable region sequence of at least 90% sequence identity to the amino acid sequence of SEQ ID NO:10. In some embodiments, the antibody comprises a heavy chain variable region sequence of the amino acid sequence of SEQ ID NO:9 and/or a light chain variable region sequence of the amino acid sequence of SEQ ID NO:10. In some embodiments, the antibody comprises a heavy chain variable region sequence of at least 90% sequence identity to the amino acid sequence of SEQ ID NO:11 and/or a light chain variable region sequence of at least 90% sequence identity to the amino acid sequence of SEQ ID NO:12. In some embodiments, the antibody comprises a heavy chain variable region sequence of the amino acid sequence of SEQ ID NO:11 and/or a light chain variable region sequence of the amino acid sequence of SEQ ID NO:12. In some embodiments, the antibody comprises a heavy chain variable region sequence of at least 90% sequence identity to the amino acid sequence of SEQ ID NO:13 and/or a light chain variable region sequence of at least 90% sequence identity to the amino acid sequence of SEQ ID NO:14. In some embodiments, the antibody comprises a heavy chain variable region sequence of the amino acid sequence of SEQ ID NO:13 and/or a light chain variable region sequence of the amino acid sequence of SEQ ID NO:14.

In some embodiments, the antibody binds to an epitope that spans over both protomers of the human IL-34 dimer. In some embodiments, the antibody neutralizes IL-34 activity. In some embodiments, the anti-IL-34 antibody is an isolated antibody that binds to human IL-34, wherein the antibody inhibits the binding between human IL-34 and human CSF-1R, and wherein the antibody neutralizes IL-34 activity.

In some embodiments, the antibody comprises (a) a HVR-H3 comprising an amino acid sequence SRGAYRFAY (SEQ ID NO: 56); (b) a HVR-L3 comprising an amino acid sequence QQSYTTPPT (SEQ ID NO: 43); and (c) a HVR-H2 comprising an amino acid sequence SITPASGDTDYADSVKG (SEQ ID NO: 54). In some embodiments, the antibody comprises (a) a HVR-H1 comprising an amino acid sequence SNYIH (SEQ ID NO: 55); (b) a HVR-H2 comprising an amino acid sequence SITPASGDTDYADSVKG (SEQ ID NO: 54); and (c) a HVR-H3 comprising an amino acid sequence SRGAYRFAY (SEQ ID NO: 56). In some embodiments, the antibody comprises (a) a HVR-L1 comprising an amino acid sequence RASQDVSTAVA (SEQ ID NO: 50); (b) a HVR-L2 comprising an amino acid sequence SASFLYS (SEQ ID NO: 53); and (c) a HVR-L3 comprising an amino acid sequence QQSYTTPPT (SEQ ID NO: 43). In some embodiments, the antibody comprises a heavy chain variable region sequence of at least 90% sequence identity to the amino acid sequence of SEQ ID NO:15 and/or a light chain variable region sequence of at least 90% sequence identity to the amino acid sequence of SEQ ID NO:16. In some embodiments, the antibody comprises a heavy chain variable region sequence of the amino acid sequence of SEQ ID NO:15 and/or a light chain variable region sequence of the amino acid sequence of SEQ ID NO:16. In some embodiments that may be combined with any of the preceding embodiments, the antibody does not inhibit the binding between human CSF-1 and human CSF-1R.

In some embodiments that may be combined with any of the preceding embodiments, the antibody is a monoclonal antibody. In some embodiments that may be combined with any of the preceding embodiments, the antibody is a human, humanized or chimeric antibody. In some embodiments that may be combined with any of the preceding embodiments, the antibody is a bispecific antibody. In some embodiments, the bispecific antibody comprises a second binding specificity to human CSF-1. In some embodiments, the bispecific antibody inhibits binding of human CSF-1 to human CSF-1R.

In some embodiments that may be combined with any of the preceding embodiments, the anti-IL-34 antibody is an antibody fragment that binds human IL-34. In some embodiments, the fragment is a Fab, Fab′, Fab′-SH, F(ab′)2, Fv or scFv fragment.

In some embodiments that may be combined with any of the preceding embodiments, the antibody is a one-armed antibody. In some embodiments that may be combined with any of the preceding embodiments, the antibody is a linear antibody. In some embodiments that may be combined with any of the preceding embodiments, the anti-IL-34 antibody is a full length IgG1 or an IgG4 antibody.

In some embodiments that may be combined with any of the preceding embodiments, the methods further comprise administering to the individual an effective amount of a CSF-1R inhibitor. In some embodiments, the CSF-1R inhibitor is a small molecule inhibitor. In some embodiments, the small molecule inhibitor is GW2580. In some embodiments, the CSF-1R inhibitor is an anti-CSF-1R antibody.

In some embodiments, the anti-CSF-1R antibody is an isolated antibody that binds human CSF-1R, which antibody binds to an epitope comprising at least one of amino acid residues Arg144, Gln248, Gln249, Ser250, Phe252, and Asn254 of human CSF-1R, wherein the position of amino acid residue is based on the position in SEQ ID NO:2, and wherein the antibody inhibits the binding between human IL-34 and human CSF-1R.

In some embodiments, the antibody binds to an epitope comprising amino acid residue Arg144 of CSF-1R, wherein the position of amino acid residue is based on the position in SEQ ID NO:2. In some embodiments, the epitope further comprises at least one of amino acid residues Arg142, Arg146, and Arg150 of human CSF-1R, and wherein the position of amino acid residues is based on the position in SEQ ID NO:2. In some embodiments, the epitope further comprises at least one of amino acid residues Ser172 and Arg192 of human CSF-1R, and wherein the position of amino acid residues is based on the position in SEQ ID NO:2. In some embodiments, the epitope further comprises at least one of amino acid residues Arg146, Met149, Arg150, Phe169, Ile170, and Gln173 of human CSF-1R, and wherein the position of amino acid residues is based on the position in SEQ ID NO:2. In some embodiments, the antibody binds to amino acids within positions 142-150 and 169-173, and wherein the position of amino acid residues is based on the position in SEQ ID NO:2.

In some embodiments, the antibody binds to an epitope comprising at least one of amino acid residues Gln248, Gln249, Ser250, Phe252, and Asn254 of human CSF-1R, wherein the position of amino acid residue is based on the position in SEQ ID NO:2. In some embodiments, the epitope further comprises amino acid residue Tyr257 of human CSF-1R, and wherein the position of amino acid residue is based on the position in SEQ ID NO:2. In some embodiments, the epitope further comprises at least one of amino acid residues Pro247, Gln258, and Lys259 of human CSF-1R, and wherein the position of amino acid residues is based on the position in SEQ ID NO:2. In some embodiments, the epitope further comprises at least one of amino acid residues Val231, Asp251, and Tyr257 of human CSF-1R, and wherein the position of amino acid residue is based on the position in SEQ ID NO:2. In some embodiments, the antibody binds to amino acid residues within positions 231, 248-252, and 254, and wherein the position of amino acid residues is based on the position in SEQ ID NO:2.

In some embodiments that may be combined with the preceding embodiments, the methods further comprise administering to the individual an effective amount of an anti-CSF-1 antibody. In some embodiments, the anti-CSF-1 antibody inhibits binding of human CSF-1 to human CSF-1R.

In some embodiments that may be combined with the preceding embodiments, the individual is a human.

In some embodiments that may be combined with the preceding embodiments, the neurological disease is selected from the group consisting of Alzheimer's disease, Parkinson's disease, Huntington's disease, amyotrophic lateral sclerosis, neuropathic pain, prion disease, spinocerebellar ataxia, spinal muscular atrophy, autism, and autism spectrum disorders. In some embodiments, the neurological disease is Alzheimer's disease. In some embodiments that may be combined with the preceding embodiments, the neurological disease is characterized by neuroinflammation and microgliosis.

Another aspect includes kits comprising a pharmaceutical composition comprising an anti-IL-34 antibody and a pharmaceutically acceptable carrier. In some embodiments, the pharmaceutical composition further comprises an inhibitor of CSF-1R. In some embodiments, the inhibitor of CSF-1R is a small molecule inhibitor. In some embodiments, the inhibitor of CSF-1R is an anti-CSF-1R antibody. In some embodiments, the kit further comprises instructions for administering an effective amount of the pharmaceutical composition to an individual for treating a neurological disease. In some embodiments, the neurological disease is selected from the group consisting of Alzheimer's disease, Parkinson's disease, Huntington's disease, amyotrophic lateral sclerosis, neuropathic pain, prion disease, spinocerebellar ataxia, spinal muscular atrophy, autism, and autism spectrum disorders. In some embodiments, the neurological disease is Alzheimer's disease. In some embodiments, the neurological disease is neuropathic pain. In some embodiments, the neurological disease is amyotrophic lateral sclerosis.

It is to be understood that one, some, or all of the properties of the various embodiments described herein may be combined to form other embodiments of the present invention. These and other aspects of the invention will become apparent to one of skill in the art.

### BRIEF DESCRIPTION OF THE FIGURES

FIGS. 1A-1B shows variable heavy (FIG. 1A) and light (FIG. 1B) chain sequences of anti-IL-34 Abs YW404.1, YW404.6, YW405.3, YW404.33, YW404.33.10, YW404.33.12, YW404.33.11, YW404.33.56, and YW404.33.93. Amino acid residues targeted for affinity-maturation for these antibodies are surrounded by a box. FIG. 1A shows the VH amino acid sequences for 404.1 (SEQ ID NO:15), 404.6 (SEQ ID NO: 68), 405.3 (SEQ ID NO:25), 404.33 (SEQ ID NO:5), 404.33.10 (SEQ ID NO:7), 404.33.12 (SEQ ID NO:11), 404.33.11 (SEQ ID NO:9), 404.33.56 (SEQ ID NO:3), and 404.33.93 (SEQ ID NO:13). CDR-H1 (SEQ ID NO: 55), CDR-H2 (SEQ ID NO: 54), and CDR-H3 (SEQ ID NO: 56) are indicated for 404.1, according to Kabat numbering. CDR-H1 (SEQ ID NO: 70), CDR-H2 (SEQ ID NO: 71), and CDR-H3 (SEQ ID NO: 72) are indicated for 404.6, according to Kabat numbering. CDR-H1 (SEQ ID NO: 73), CDR-H2 (SEQ ID NO: 74), and CDR-H3 (SEQ ID NO: 75) are indicated for 405.3, according to Kabat numbering. CDR-H1 (SEQ ID NO: 59), CDR-H2 (SEQ ID NO: 51), and CDR-H3 (SEQ ID NO: 33) are indicated for 404.33, according to Kabat numbering. CDR-H1 (SEQ ID NO: 59), CDR-H2 (SEQ ID NO: 51), and CDR-H3 (SEQ ID NO: 33) are indicated for 404.33.10, according to Kabat numbering. CDR-H1 (SEQ ID NO: 59), CDR-H2 (SEQ ID NO: 51), and CDR-H3 (SEQ ID NO: 33) are indicated for 404.33.12, according to Kabat numbering. CDR-H1 (SEQ ID NO: 59), CDR-H2 (SEQ ID NO: 51), and CDR-H3 (SEQ ID NO: 33) are indicated for 404.33.11, according to Kabat numbering. CDR-H1 (SEQ ID NO: 59), CDR-H2 (SEQ ID NO: 52), and CDR-H3 (SEQ ID NO: 33) are indicated for 404.33.56, according to Kabat numbering. CDR-H1 (SEQ ID NO: 59), CDR-H2 (SEQ ID NO: 51), and CDR-H3 (SEQ ID NO: 32) are indicated for 404.33.93, according to Kabat numbering. CDR-H1 (SEQ ID NO: 31), CDR-H2 (SEQ ID NO: 35), and CDR-H3 (SEQ ID NO: 56) are indicated for 404.1, according to Chothia numbering. CDR-H3 (SEQ ID NO: 72) is indicated for 404.6, according to Chothia numbering. CDR-H3 (SEQ ID NO: 75) is indicated for 405.3, according to Chothia numbering. CDR-H1 (SEQ ID NO: 30), CDR-H2 (SEQ ID NO: 36), and CDR-H3 (SEQ ID NO: 33) are indicated for 404.33, according to Chothia numbering. CDR-H1 (SEQ ID NO: 30), CDR-H2 (SEQ ID NO: 36), and CDR-H3 (SEQ ID NO: 33) are indicated for 404.33.10, according to Chothia numbering. CDR-H1 (SEQ ID NO: 30), CDR-H2 (SEQ ID NO: 36), and CDR-H3 (SEQ ID NO: 33) are indicated for 404.33.12, according to Chothia numbering. CDR-H1 (SEQ ID NO: 30), CDR-H2 (SEQ ID NO: 36), and CDR-H3 (SEQ ID NO: 33) are indicated for 404.33.11, according to Chothia numbering. CDR-H1 (SEQ ID NO: 30), CDR-H2 (SEQ ID NO: 37), and CDR-H3 (SEQ ID NO: 33) are indicated for 404.33.56, according to Chothia numbering. CDR-H1 (SEQ ID NO: 30), CDR-H2 (SEQ ID NO: 36), and CDR-H3 (SEQ ID NO: 32) are indicated for 404.33.93, according to Chothia numbering. CDR-H1 (SEQ ID NO: 60), CDR-H2 (SEQ ID NO: 63), and CDR-H3 (SEQ ID NO: 29) are indicated for 404.1, according to Contact numbering. CDR-H1 (SEQ ID NO: 57), CDR-H2 (SEQ ID NO: 61), and CDR-H3 (SEQ ID NO: 28) are indicated for 404.33, according to Contact numbering. CDR-H1 (SEQ ID NO: 57), CDR-H2 (SEQ ID NO: 61), and CDR-H3 (SEQ ID NO: 28) are indicated for 404.33.10, according to Contact numbering. CDR-H1 (SEQ ID NO: 57), CDR-H2 (SEQ ID NO: 61), and CDR-H3 (SEQ ID NO: 28) are indicated for 404.33.12, according to Contact numbering. CDR-H1 (SEQ ID NO: 57), CDR-H2 (SEQ ID NO: 61), and CDR-H3 (SEQ ID NO: 28) are indicated for 404.33.11, according to Contact numbering. CDR-H1 (SEQ ID NO: 57), CDR-H2 (SEQ ID NO: 62), and CDR-H3 (SEQ ID NO: 28) are indicated for 404.33.56, according to Contact numbering. CDR-H1 (SEQ ID NO: 57), CDR-H2 (SEQ ID NO: 61), and CDR-H3 (SEQ ID NO: 27) are indicated for 404.33.93, according to Contact numbering. FIG. 1B shows the VL amino acid sequences for 404.1 (SEQ ID NO:16), 404.6 (SEQ ID NO: 69), 405.3 (SEQ ID NO:26), 404.33 (SEQ ID NO:6), 404.33.10 (SEQ ID NO:8), 404.33.12 (SEQ ID NO:12), 404.33.11 (SEQ ID NO:10), 404.33.56 (SEQ ID NO:4), and 404.33.93 (SEQ ID NO:14). CDR-L1 (SEQ ID NO: 50), CDR-L2 (SEQ ID NO: 53), and CDR-L3 (SEQ ID NO: 43) are indicated for 404.1, according to Kabat numbering. CDR-L1 (SEQ ID NO: 50), CDR-L2 (SEQ ID NO: 53), and CDR-L3 (SEQ ID NO: 43) are indicated for 404.6, according to Kabat numbering. CDR-L1 (SEQ ID NO: 76), CDR-L2 (SEQ ID NO: 77), and CDR-L3 (SEQ ID NO: 78) are indicated for 405.3, according to Kabat numbering. CDR-L1 (SEQ ID NO: 50), CDR-L2 (SEQ ID NO: 53), and CDR-L3 (SEQ ID NO: 43) are indicated for 404.33, according to Kabat numbering. CDR-L1 (SEQ ID NO: 50), CDR-L2 (SEQ ID NO: 53), and CDR-L3 (SEQ ID NO: 49) are indicated for 404.33.10, according to Kabat numbering. CDR-L1 (SEQ ID NO: 50), CDR-L2 (SEQ ID NO: 53), and CDR-L3 (SEQ ID NO: 45) are indicated for 404.33.12, according to Kabat numbering. CDR-L1 (SEQ ID NO: 50), CDR-L2 (SEQ ID NO: 53), and CDR-L3 (SEQ ID NO: 47) are indicated for 404.33.11, according to Kabat numbering. CDR-L1 (SEQ ID NO: 50), CDR-L2 (SEQ ID NO: 53), and CDR-L3 (SEQ ID NO: 39) are indicated for 404.33.56, according to Kabat numbering. CDR-L1 (SEQ ID NO: 50), CDR-L2 (SEQ ID NO: 53), and CDR-L3 (SEQ ID NO: 41) are indicated for 404.33.93, according to Kabat numbering. CDR-L1 (SEQ ID NO: 50), CDR-L2 (SEQ ID NO: 53), and CDR-L3 (SEQ ID NO: 43) are indicated for 404.1, according to Chothia numbering. CDR-L1 (SEQ ID NO: 50), CDR-L2 (SEQ ID NO: 53), and CDR-L3 (SEQ ID NO: 43) are indicated for 404.6, according to Chothia numbering. CDR-L1 (SEQ ID NO: 76), CDR-L2 (SEQ ID NO: 77), and CDR-L3 (SEQ ID NO: 78) are indicated for 405.3, according to Chothia numbering. CDR-L1 (SEQ ID NO: 50), CDR-L2 (SEQ ID NO: 53), and CDR-L3 (SEQ ID NO: 43) are indicated for 404.33, according to Chothia numbering. CDR-L1 (SEQ ID NO: 50), CDR-L2 (SEQ ID NO: 53), and CDR-L3 (SEQ ID NO: 49) are indicated for 404.33.10, according to Chothia numbering. CDR-L1 (SEQ ID NO: 50), CDR-L2 (SEQ ID NO: 53), and CDR-L3 (SEQ ID NO: 45) are indicated for 404.33.12, according to Chothia numbering. CDR-L1 (SEQ ID NO: 50), CDR-L2 (SEQ ID NO: 53), and CDR-L3 (SEQ ID NO: 47) are indicated for 404.33.11, according to Chothia numbering. CDR-L1 (SEQ ID NO: 50), CDR-L2 (SEQ ID NO: 53), and CDR-L3 (SEQ ID NO: 39) are indicated for 404.33.56, according to Chothia numbering. CDR-L1 (SEQ ID NO: 50), CDR-L2 (SEQ ID NO: 53), and CDR-L3 (SEQ ID NO: 41) are indicated for 404.33.93, according to Chothia numbering. CDR-L1 (SEQ ID NO: 58), CDR-L2 (SEQ ID NO: 34), and CDR-L3 (SEQ ID NO: 42) are indicated for 404.1, according to Contact numbering. CDR-L1 (SEQ ID NO: 58), CDR-L2 (SEQ ID NO: 34), and CDR-L3 (SEQ ID NO: 42) are indicated for 404.6, according to Contact numbering. CDR-L1 (SEQ ID NO: 58), CDR-L2 (SEQ ID NO: 34), and CDR-L3 (SEQ ID NO: 42) are indicated for 404.33, according to Contact numbering. CDR-L1 (SEQ ID NO: 58), CDR-L2 (SEQ ID NO: 34), and CDR-L3 (SEQ ID NO: 48) are indicated for 404.33.10, according to Contact numbering. CDR-L1 (SEQ ID NO: 58), CDR-L2 (SEQ ID NO: 34), and CDR-L3 (SEQ ID NO: 44) are indicated for 404.33.12, according to Contact numbering. CDR-L1 (SEQ ID NO: 58), CDR-L2 (SEQ ID NO: 34), and CDR-L3 (SEQ ID NO: 46) are indicated for 404.33.11, according to Contact numbering. CDR-L1 (SEQ ID NO: 58), CDR-L2 (SEQ ID NO: 34), and CDR-L3 (SEQ ID NO: 38) are indicated for 404.33.56, according to Contact numbering. CDR-L1 (SEQ ID NO: 58), CDR-L2 (SEQ ID NO: 34), and CDR-L3 (SEQ ID NO: 40) are indicated for 404.33.93, according to Contact numbering. The heavy chain framework region sequences between Kabat HVRs are FR1 sequence (SEQ ID NO:17), FR2 sequence (SEQ ID NO:18), FR3 (SEQ ID NO:19), and FR4 (SEQ ID NO:20) shown in FIG. 1A. The light chain framework region sequences between Kabat HVRs are FR1 sequence (SEQ ID NO:21), FR2 sequence (SEQ ID NO:22), FR3 sequence (SEQ ID NO:23), and FR4 sequence (SEQ ID NO:24) shown in FIG. 1B. The anti-IL-34 Abs YW404.1, YW404.6, YW405.3, YW404.33, YW404.33.10, YW404.33.12, YW404.33.11, YW404.33.56, and YW404.33.93 described in FIG. 1 are described in PCT/US13/24998 (Publ No. WO/2013/119716).

FIG. 2 shows representative images of microglia in CX3CR1-GFP mice after treatment with anti-IL-34 antibody, anti-IL-34 antibody plus small molecule inhibitor GW2580, or anti-gp120 control antibody.

FIGS. 3A-3D shows microglia density (FIG. 3A), average soma size (FIG. 3B), cell perimeter (FIG. 3C), and average microglia size (FIG. 3D) in CX3CR1-GFP mice after treatment with anti-IL-34 antibody, anti-IL-34 antibody plus small molecule inhibitor GW2580, or anti-gp120 control antibody.

FIG. 4 shows representative images of microglia in CX3CR1-GFP mice after treatment with anti-IL-34 antibody IP plus small molecule inhibitor GW2580 PO, or anti-gp120 control antibody IP plus vehicle (methylcellulose Tween-80 (MCT)) PO.

FIG. 5 shows microglia density in CX3CR1-GFP mice after treatment with anti-IL-34 antibody plus small molecule inhibitor GW2580 or anti-gp120 control antibody IP plus MCT PO.

FIGS. 6A-6B shows Iba1 immunohistochemistry (FIG. 6A) and Iba1-positive cell counts (FIG. 6B) in microglia in CX3CR1-GFP mice after treatment with anti-IL-34 antibody, anti-IL-34 antibody plus small molecule inhibitor GW2580, or anti-gp120 control antibody. Iba1-positive cell counts confirm that anti-IL-34 antibody and anti-IL-34 antibody plus small molecule inhibitor GW2580 effectively deplete microglia and don't simply cause a loss of CX3CR1-GFP expression.

FIG. 7 shows that no change in Iba1 expression was seen after anti-IL-34 antibody or anti-IL-34 antibody plus small molecule inhibitor GW2580 treatments, suggesting that microglia depletion does not result in activation of the remaining microglia.

FIGS. 8A-8B shows GFAP immunohistochemistry (FIG. 8A) and GFAP positive cell counts (FIG. 8B) in microglia in CX3CR1-GFP mice after treatment with anti-IL-34 antibody, anti-IL-34 antibody plus small molecule inhibitor GW2580, or anti-gp120 control antibody. Despite the loss of significant numbers of microglia after anti-IL-34 antibody or anti-IL-34 antibody plus small molecule inhibitor GW2580 treatments, immunohistochemistry for GFAP shows no change in astrocytes, indicating that microglia depletion did not cause an astrocyte response.

FIG. 9 shows representative images of microglia in CX3CR1-GFP mice after treatment with anti-IL-34 antibody, anti-CSF1 antibody, anti-IL-34 antibody plus anti-CSF1 antibody, anti-IL-34 antibody plus small molecule inhibitor GW2580, or anti-gp120 control antibody.

FIGS. 10A-10D shows microglia density (FIG. 10A), average soma size (FIG. 10B), cell perimeter (FIG. 10C), and average microglia size (FIG. 10D) in CX3CR1-GFP mice after treatment with anti-IL-34 antibody, ant-CSF1 antibody, anti-IL-34 antibody plus anti-CSF1 antibody, anti-IL-34 antibody plus small molecule inhibitor GW2580, or anti-gp120 control antibody.

FIG. 11 shows representative images of microglia in cortical gray matter from CX3CR1-GFP mice after treatment with Compound X milled into chow, or control chow.

FIG. 12 shows microglia density in cortical gray matter from CX3CR1-GFP mice after treatment with Compound X milled into chow, or control chow.

FIG. 13 shows representative images of microglia in cortical gray matter from CX3CR1-GFP mice after treatment with anti-IL-34 antibody, anti-CSF1 antibody, anti-IL-34 antibody plus anti-CSF1 antibody, or anti-gp120 control antibody.

FIG. 14 shows microglia density in cortical gray matter from CX3CR1-GFP mice after treatment with anti-IL-34 antibody, anti-CSF1 antibody, anti-IL-34 antibody plus anti-CSF1 antibody, or anti-gp120 control antibody.

FIG. 15 shows representative images of microglia in white matter from the corpus callosum of CX3CR1-GFP mice after treatment with anti-IL-34 antibody, anti-CSF1 antibody, anti-IL-34 antibody plus anti-CSF1 antibody, or anti-gp120 control antibody. *p<0.05.

FIG. 16 shows microglia density in white matter from the corpus callosum of CX3CR1-GFP mice after treatment with anti-IL-34 antibody, anti-CSF1 antibody, anti-IL-34 antibody plus anti-CSF1 antibody, or anti-gp120 control antibody. *p<0.05, **p<0.00005.

FIG. 17 shows representative images of microglia in white matter from the hippocampal fimbria of CX3CR1-GFP mice after treatment with anti-IL-34 antibody, anti-CSF1 antibody, anti-IL-34 antibody plus anti-CSF1 antibody, or anti-gp120 control antibody.

FIG. 18 shows microglia density in white matter from the hippocampal fimbria of CX3CR1-GFP mice after treatment with anti-IL-34 antibody, anti-CSF1 antibody, anti-IL-34 antibody plus anti-CSF1 antibody, or anti-gp120 control antibody. *p<0.05.

FIG. 19 shows representative images of microglia in the hippocampus of CX3CR1-GFP mice after treatment with anti-IL-34 antibody, anti-CSF1 antibody, anti-IL-34 antibody plus anti-CSF1 antibody, or anti-gp120 control antibody.

FIG. 20 shows the percent microglia-labeled area in the hippocampus of CX3CR1-GFP mice after treatment with anti-IL-34 antibody, anti-CSF1 antibody, anti-IL-34 antibody plus anti-CSF1 antibody, or anti-gp120 control antibody. *p<0.05.

FIGS. 21A-21D shows the association of plaques and microglia in the PS2APP Alzheimer's disease mouse model. FIG. 21A shows representative images of dense-core amyloid plaques, blood vessels, and microglia in 13-32 week old PS2APP^{+/+} CX3CR1-GFP mice. FIG. 21B shows microglia density of plaque-associated microglia in 18-52 week old PS2APP^{+/+} CX3CR1-GFP mice. FIG. 21C shows the total microglia in 12-52 week old PS2APP^{+/+} and PS2APP^{-/-} mice.

FIG. 21D shows microglia proliferation in 12-52 week old PS2APP^{+/+} and PS2APP^{-/-} mice.

FIGS. 22A-22C shows the association of plaques and neurons/synapses in the PS2APP Alzheimer's disease mouse model. FIG. 22A shows representative images of dense-core amyloid plaques, blood vessels, and neurons/synapses in 13-32 week old PS2APP^{+/+} GFP-M mice. FIG. 22B shows dendritic spine density near to (spine density on dendritc segment within field of view with plaque), and away from (spine density on dendrite segment at least 100 microns from nearest plaque), plaques in 13-100 week old PS2APP^{+/+} and PS2APP^{-/-} mice. FIG. 22C shows plaque density and predicted relative synapse density in 12-100 week old PS2APP^{+/+} mice.

FIGS. 23A-23E shows the treatment regimen and results from depleting microglia in the PS2APP Alzheimer's disease mouse model. FIG. 23A shows a timeline of the treatment regimen for PS2APP^{+/+} CX3CR1-GFP mice treated with anti-IL-34 antibody plus small molecule inhibitor GW2580, or a vehicle control (anti-gp120 control antibody plus MCT vehicle). FIG. 23B shows representative images of microglia in PS2APP^{+/+} CX3CR1-GFP mice treated with anti-IL-34 antibody plus small molecule inhibitor GW2580, or a vehicle control (anti-gp120 control antibody plus MCT vehicle) for four weeks. FIG. 23C shows microglia density in PS2APP^{+/+} CX3CR1-GFP mice treated with anti-IL-34 antibody plus small molecule inhibitor GW2580, or a vehicle control (anti-gp120 control antibody plus MCT vehicle) using the indicated treatment regimen. FIG. 23D shows representative images of microglia, plaques, and neurons in PS2APP^{+/+} CX3CR1-GFP mice after treatment with anti-IL-34 antibody plus small molecule inhibitor GW2580, or a vehicle control (anti-gp120 control antibody plus MCT vehicle). Plaques were labeled with methoxy-X04, and neurons were labeled by in utero electroporation of a ds-red expressing plasmid at E16 to label layer 2/3 pyramidal neurons in the somatosensory cortex. FIG. 23E shows the spine density ratio in PS2APP^{+/+} CX3CR1-GFP mice after treatment with anti-IL-34 antibody plus small molecule inhibitor GW2580 (depletion), or a vehicle control (anti-gp120 control antibody plus MCT vehicle) using the indicated treatment regimen. *p<0.05, **p<0.001.

FIG. 24 shoes images and quantitation of plaque density in PS2APP^{+/+} CX3CR1-GFP mice treated with anti-IL-34 antibody plus small molecule inhibitor GW2580 (depletion) or a vehicle control (anti-gp120 control antibody plus MCT vehicle).

FIG. 25 shows plaque size in mice treated with anti-IL-34 antibody plus small molecule inhibitor GW2580 (depletion) or a vehicle control (anti-gp120 control antibody plus MCT vehicle).

FIG. 26 shows immunohistochemistry for GFAP, a marker for astrocytes, in mice treated with anti-IL-34 antibody plus small molecule inhibitor GW2580 (depleted) or a vehicle control (anti-gp120 control antibody plus MCT vehicle).

FIG. 27 shows horizontal activity in an open field task, which measures general locomotor behavior, for mice treated with anti-IL-34 antibody plus small molecule inhibitor GW2580 (depletion) or a vehicle control (anti-gp120 control antibody plus MCT vehicle).

FIG. 28 shows immunohistochemistry for Iba1 and confirms microglia depletion seen by GFP positive cell counts (*p=0.005) in mice treated with anti-IL-34 antibody plus small molecule inhibitor GW2580 (depleted) or a vehicle control (anti-gp120 control antibody plus MCT vehicle).

### DETAILED DESCRIPTION OF EMBODIMENTS OF THE INVENTION

### I. Definitions

The terms “anti-IL-34 antibody” and “an antibody that binds to IL-34” refer to an antibody that is capable of binding IL-34 with sufficient affinity such that the antibody is useful as a diagnostic and/or therapeutic agent in targeting IL-34. In some embodiments, the extent of binding of an anti-IL-34 antibody to an unrelated, non-IL-34 protein is less than about 10% of the binding of the antibody to IL-34 as measured, e.g., by a BIACORE assay or a BLI assay. In some embodiments, an antibody that binds to IL-34 has a dissociation constant (Kd) of ≤1 μM, ≤500 nM, ≤250 nM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³M, e.g., from 10⁻⁹ M to 10⁻¹³ M). In some embodiments, an anti-IL-34 antibody binds to an epitope of IL-34 that is conserved among IL-34 from different species.

The term “IL-34,” as used herein, refers to any native IL-34 from any vertebrate source, including mammals such as primates (e.g., humans) and rodents (e.g., mice and rats), unless otherwise indicated. The term encompasses “full-length,” unprocessed IL-34 as well as any form of IL-34 that results from processing in the cell. The term also encompasses naturally occurring variants of IL-34, e.g., splice variants or allelic variants. The amino acid sequence of an exemplary human IL-34 is shown in SEQ ID NO:1. In some embodiments, the human IL-34 comprises the amino acid sequence shown in SEQ ID NO:1, wherein amino acid Q at position 81 is deleted.

| (SEQ ID NO: 1) | |
|---|---|
| 1 | MPRGFTWLRY LGIFLGVALG NEPLEMWPLT QNEECTVTGF LRDKLQYRSR LQYMKHYFPI |
| 61 | NYKISVPYEG VFRIANVTRL QRAQVSEREL RYLWVLVSLS ATESVQDVLL EGHPSWKYLQ |
| 121 | EVETLLLNVQ QGLTDVEVSP KVESVLSLLN APGPNLKLVR PKALLDNCFR VMELLYCSCC |
| 181 | KQSSVLNWQD CEVPSPQSCS PEPSLQYAAT QLYPPPPWSP SSPPHSTGSV RPVRAQGEGL |
| 241 | LP. |

The terms “anti-CSF-1 antibody” and “an antibody that binds to CSF-1” refer to an antibody that is capable of binding CSF-1 with sufficient affinity such that the antibody is useful as a diagnostic and/or therapeutic agent in targeting CSF-1. In some embodiments, the extent of binding of an anti-CSF-1 antibody to an unrelated, non-CSF-1 protein is less than about 10% of the binding of the antibody to CSF-1 as measured, e.g., by a BIACORE assay or a BLI assay. In some embodiments, an antibody that binds to CSF-1 has a dissociation constant (Kd) of ≤1 μM, ≤500 nM, ≤250 nM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M). In some embodiments, an anti-CSF-1 antibody binds to an epitope of CSF-1 that is conserved among CSF-1 from different species.

The term “CSF-1,” as used herein, refers to any native CSF-1 from any vertebrate source, including mammals such as primates (e.g., humans) and rodents (e.g., mice and rats), unless otherwise indicated. The term encompasses “full-length,” unprocessed CSF-1 as well as any form of CSF-1 that results from processing in the cell. The term also encompasses naturally occurring variants of CSF-1, e.g., splice variants or allelic variants. An exemplary human CSF-1 is described in Takahashi et al., Biochem. Biophys. Res. Commun. 161 (2), 892-901 (1989).

The terms “anti-CSF-1R antibody” and “an antibody that binds to CSF-1R” refer to an antibody that is capable of binding CSF-1R with sufficient affinity such that the antibody is useful as a diagnostic and/or therapeutic agent in targeting CSF-1R. In some embodiments, the extent of binding of an anti-CSF-1R antibody to an unrelated, non-CSF-1R protein is less than about 10% of the binding of the antibody to CSF-1R as measured, e.g., by a BIACORE assay or a BLI assay. In some embodiments, an antibody that binds to CSF-1R has a dissociation constant (Kd) of ≤1 μM, ≤500 nM, ≤250 nM, ≤100 nM, ≤10 nM, ≤1 nM, ≤0.1 nM, ≤0.01 nM, or ≤0.001 nM (e.g., 10⁻⁸ M or less, e.g., from 10⁻⁸M to 10⁻¹³M, e.g., from 10⁻⁹M to 10⁻¹³ M). In some embodiments, an anti-CSF-1R antibody binds to an epitope of CSF-1R that is conserved among IL-34 from different species.

The term “CSF-1R” or “CSF1R” as used herein, refers to any native CSF-1R from any vertebrate source, including mammals such as primates (e.g., humans) and rodents (e.g., mice and rats), unless otherwise indicated. The term encompasses “full-length,” unprocessed CSF-1R as well as any form of CSF-1R that results from processing in the cell. The term also encompasses naturally occurring variants of CSF-1R, e.g., splice variants or allelic variants. The amino acid sequence of an exemplary human CSF-1R is shown in SEQ ID NO:2.

(SEQ ID NO: 2)
mgpgvlllll vatawhgqgi pviepsvpel vvkpgatvtl
rcvgngsvew dgppsphwtl ysdgsssils tnnatfqntg
tyrctepgdp lggsaaihly vkdparpwnv laqevvvfed
qdallpcllt dpvleagvsl vrvrgrplmr htnysfspwh
gftihrakfi qsqdyqcsal mggrkvmsis irlkvqkvip
gppaltivpa elvrirgeaa qivcsassvd vnfdvflqhn
ntklaipqqs dfhnnryqkv ltlnldqvdf qhagnyscva
snvqgkhsts mffrvvesay lnlsseqnli qevtvgegln
lkvmveaypg lqgfnwtylg pfsdhqpepk lanattkdty
rhtftlslpr lkpseagrys flarnpggwr altfeltlry
ppevsviwtf ingsgtllca asgypqpnvt wlqcsghtdr
cdeaqvlqvw ddpypevlsq epfhkvtvqs lltvetlehn
qtyecrahns vgsgswafip isagahthpp deflftpvvv
acmsimalll lllllllyky kqkpkyqvrw kiiesyegns
ytfidptqlp ynekwefprn nlqfgktlga gafgkvveat
afglgkedav lkvavkmlks tahadekeal mselkimshl
gqhenivnll gacthggpvl viteyccygd llnflrrkae
amlgpslspg qdpeggvdyk nihlekkyvr rdsgfssqgv
dtyvemrpvs tssndsfseq dldkedgrpl elrdllhfss
qvaqgmafla skncihrdva arnvlltngh vakigdfgla
rdimndsnyi vkgnarlpvk wmapesifdc vytvqsdvws
ygillweifs lglnpypgil vnskfyklvk dgyqmaqpaf
apkniysimq acwalepthr ptfqqicsfl qeqaqedrre
rdytnlpsss rsggsgssss eleeessseh ltcceqgdia
QPLLQPNNYQ FC

A therapeutic agent according to this invention includes an agent that can bind to the target identified herein above, such as a polypeptide(s) (e.g., an antibody, an immunoadhesin or a peptibody), an aptamer or a small molecule that can bind to a protein or a nucleic acid molecule that can bind to a nucleic acid molecule encoding a target identified herein (i.e., siRNA).

The term “CSF1-R pathway inhibitor” refers to a therapeutic agent that inhibits CSF1-R signaling. In one embodiment, the CSF1-R pathway inhibitor binds to CSF-1, IL-34, CSF1-R or CSF-1 and IL-34. In one embodiment, the agent that binds CSF-1, IL-34 or CSF-1 and IL-34 inhibits the binding of such protein(s) to CSF1-R. In another embodiment, the agent that binds CSF1-R inhibits the binding of CSF1-R to IL-34 and CSF-1. In one embodiment, a reduction in kinase activity of CSF1-R indicates a reduction in CSF-1R signaling. In one embodiment, the CSF1-R pathway inhibitor is an antibody of the present disclosure. In another embodiment, the CSF-1R pathway inhibitor is a small molecule inhibitor of CSF1-R. In another embodiment, the CSF1-R pathway inhibitor is a CSF1-R extracellular domain fused to an Fc.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. An HVR as used herein can comprise residues located within positions 24-36 (for L1), 46-56 (for L2), 89-97 (for L3), 26-35B (for H1), 47-65 (for H2), and 93-102 (for H3). For example, an HVR can include residues in positions described previously:

A) 24-34 (L1), 50-56 (L2), 89-97 (L3), 26-32 (H1), 52-56 (H2), and 95-102 (H3) (Chothia and Lesk, J. Mol. Biol. 196:901-917 (1987);

B) 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, Md. (1991); and

C) 30-36 (L1), 46-55 (L2), 89-96 (L3), 30-35 (H1), 47-58 (H2), 93-101 (H3) (MacCallum et al. J. Mol. Biol. 262:732-745 (1996).

Unless otherwise indicated, HVR residues and other residues in the variable domain (e.g., FR residues) are numbered herein according to Kabat et al., supra.

Unless otherwise indicated, HVR residues and other residues in the variable domain (e.g., FR residues) are numbered herein according to Kabat et al., supra.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human HVRs and amino acid residues from human FRs. In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

A “naked antibody” refers to an antibody that is not conjugated to a heterologous moiety (e.g., a cytotoxic moiety) or radiolabel. The naked antibody may be present in a pharmaceutical formulation.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-IL-34 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “effective amount” of an agent, e.g., a pharmaceutical formulation, refers to an amount effective, at dosages and for periods of time necessary, to achieve the desired therapeutic or prophylactic result.

As is understood in the clinical context, an effective amount of a therapeutic agent (e.g., an antibody provided herein), drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

### II. Compositions and Methods

In one aspect, the invention provides a method of treating a neurological disease in an individual, treating an individual exhibiting one or more symptoms of a neurological disease, or reducing the density of microglia in the brain of an individual, by administering an anti-IL-34 antibody.

A. Exemplary Antibodies and Inhibitors

### Anti-IL-34 Antibodies

In one aspect, the invention provides a method of treating a neurological disease in an individual, treating an individual exhibiting one or more symptoms of a neurological disease, or reducing the density of microglia in the brain of an individual, comprising administering to the individual an effective amount of an anti-IL-34 antibody. In some embodiments, the anti-IL-34 antibody is an isolated antibody that binds to IL-34 (e.g., human IL-34). In some embodiments, the anti-IL-34 antibody is clone YW404.33.1. In some embodiments, the anti-IL-34 antibody isotype is mouse IgG2A.

The anti-IL-34 antibodies described herein may have one or more of the following characteristics: (i) inhibition of binding of IL-34 (e.g., human IL-34) to CSF-1R (e.g., human CSF-1R); (ii) neutralization of IL-34 activity (e.g., human IL-34 activity); (iii) inhibition of IL-34 induced proliferation of peripheral blood mononuclear cells; (iv) binding to a dimer of IL-34 (e.g., human IL-34); (v) binding to an epitope that spans over both protomers of IL-34 (e.g., human IL-34); (vi) no inhibition of binding of CSF-1 (e.g., human CSF-1) to CSF-1R (e.g., human CSF-1R). In some embodiments, the extent of binding of an anti-IL-34 antibody to an unrelated, non-IL-34 protein is less than about 10% of the binding of the antibody to IL-34 as measured, e.g., by a BIACORE assay or a biolayer interferometry (BLI) assay. In some embodiments, the antibody that binds to IL-34 has a dissociation constant (Kd) of ≤1 μM, ≤500 nM, ≤250 nM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³M, e.g., from 10⁻⁹M to 10⁻¹³ M). In some embodiments, the anti-IL-34 antibody has a Kd value of less than about 500 nM. In some embodiments, the anti-IL-34 antibody has a Kd value of less than about 100 nM or 10 nM. In some embodiments, the anti-IL-34 antibody has a Kd value of less than about 1 nM. In some embodiments, the IL-34 antibody has a Kd value of less than about 100 pM. In some embodiments, an anti-IL-34 antibody has a Kd of about 100-200 pM, about 100-500 pM, about 100 pM-1 nM, or of about 1 nM-50 nM. In some embodiments, an anti-IL-34 antibody has a Kd of about 17 nM. In some embodiments, an anti-IL-34 antibody has a Kd of about 120 nM. In some embodiments, the anti-IL-34 antibody binds to an epitope of IL-34 that is conserved among IL-34 from different species.

In one aspect, provided herein is an anti-IL-34 antibody, which binds to an epitope comprising at least any one of one, two, three, four, five, six, seven, eight, nine, ten, eleven, twelve, thirteen, fourteen, fifteen, or sixteen, or seventeen of amino acid residues Glu103, Leu109, Gln106, Asn150, Leu127, Asn128, Ser184, Leu186, Asn187, Lys44, Glu121, Asp107, Glu111, Ser104, Gln120, Trp116, and Asn61 of a human IL-34. In one aspect, provided herein is an anti-IL-34 antibody, which binds to an epitope comprising at least one of amino acid residues from Glu103 to Asn150 of a human IL-34. In one aspect, provided herein is an anti-IL-34 antibody, which binds to an epitope comprising at least any one of one, two, or three, or four of amino acid residues Glu103, Leu109, Gln106, and Asn150 of a human IL-34. In any of the aspects above, the anti-IL-34 antibody may bind to an epitope further comprising at least any one of one, two, three, four, five, six, or seven, or eight of amino acid residues Ser100, Glu123, Trp116, Thr124, Leu127, Asn128, Gln131, and Thr134 of a human IL-34. In some embodiments, the anti-IL-34 antibody binds to amino acids within positions 100-108, 116-134, 109 and 150 of a human IL-34. In some embodiments, the anti-IL-34 antibody inhibits binding between human IL-34 and human CSF-1R. In some embodiments, the anti-IL-34 antibody neutralizes human IL-34 activity. In some embodiments, the anti-IL-34 antibody binds to a dimer of human IL-34. In some embodiments, the anti-IL-34 antibody binds to an epitope that spans both protomers of human IL-34. In some embodiments, the anti-IL-34 antibody is a monoclonal antibody. In some embodiments, the anti-IL-34 antibody is a human, humanized, or chimeric antibody. In some embodiments, the anti-IL-34 antibody is an antibody fragment that binds to human IL-34. As used herein, the residue position herein corresponds to the residue position in SEQ ID NO:1.

In one aspect, provided herein is an anti-IL-34 antibody, which binds to an epitope comprising at least any one of one, two, three, four, five, six, seven, eight, nine, ten, eleven, twelve, thirteen, fourteen, fifteen, or sixteen, or seventeen of amino acid residues Glu103, Leu109, Gln106, Asn150, Leu127, Asn128, Ser184, Leu186, Asn187, Lys44, Glu121, Asp107, Glu111, Ser104, Gln120, Trp116, and Asn61 of a human IL-34. In one aspect, provided herein is an anti-IL-34 antibody, which binds to an epitope comprising at least any one of one, two, three, four, or five, or six of amino acid residues Asn128, Ser184, Leu186, Asn187, Lys44, and Glu121 of a human IL-34. In any of the aspects above, the anti-IL-34 antibody may bind to an epitope further comprising at least any one of one, two, three, four, or five, or six of amino acid residues Phe40, Asp43, Leu125, Gln189, Thr36, and Val185 of a human IL-34. In some embodiments, the anti-IL-34 antibody binds to amino acids within positions 36-44, 121-128, and 184-187 of a human IL-34. In some embodiments, the anti-IL-34 antibody inhibits binding between human IL-34 and human CSF-1R. In some embodiments, the anti-IL-34 antibody neutralizes human IL-34 activity. In some embodiments, the anti-IL-34 antibody binds to a dimer of human IL-34. In some embodiments, the anti-IL-34 antibody binds to an epitope that spans both protomers of human IL-34. In some embodiments, the anti-IL-34 antibody is a monoclonal antibody. In some embodiments, the anti-IL-34 antibody is a human, humanized, or chimeric antibody. In some embodiments, the anti-IL-34 antibody is an antibody fragment that binds to human IL-34. As used herein, the residue position herein corresponds to the residue position in SEQ ID NO:1.

In one aspect, provided herein is an anti-IL-34 antibody that binds to an epitope comprising at least one of amino acid residues from Glu103-Leu127 of a human IL-34. In one aspect, provided herein is an anti-IL-34 antibody that binds to an epitope comprising at least any one of one, two, three, four, five, six, or seven, or eight of amino acid residues Asp107, Glu111, Ser104, Gln120, Glu103, Leu109, Trp116, and Asn61 of a human IL-34. In any of the aspects provided above, the antibody may bind to an epitope which further comprises at least any one of one, two, three, four, five, six, seven, or eight, or nine of amino acid residues Pro152, Val108, Leu110, Gln106, Glu123, Leu127, Lys117, Ile60 and Lys55 of a human IL-34. In some embodiments, the antibody binds to amino acids within positions 55-61, 100-108, 109, 111-127 and 152 of a human IL-34. In some embodiments, the anti-IL-34 antibody inhibits binding between human IL-34 and human CSF-1R. In some embodiments, the anti-IL-34 antibody neutralizes human IL-34 activity. In some embodiments, the anti-IL-34 antibody binds to a dimer of human IL-34. In some embodiments, the anti-IL-34 antibody binds to an epitope that spans both protomers of human IL-34. In some embodiments, the anti-IL-34 antibody is a monoclonal antibody. In some embodiments, the anti-IL-34 antibody is a human, humanized, or chimeric antibody. In some embodiments, the anti-IL-34 antibody is an antibody fragment that binds to human IL-34. As used herein, the residue position herein corresponds to the residue position in SEQ ID NO:1.

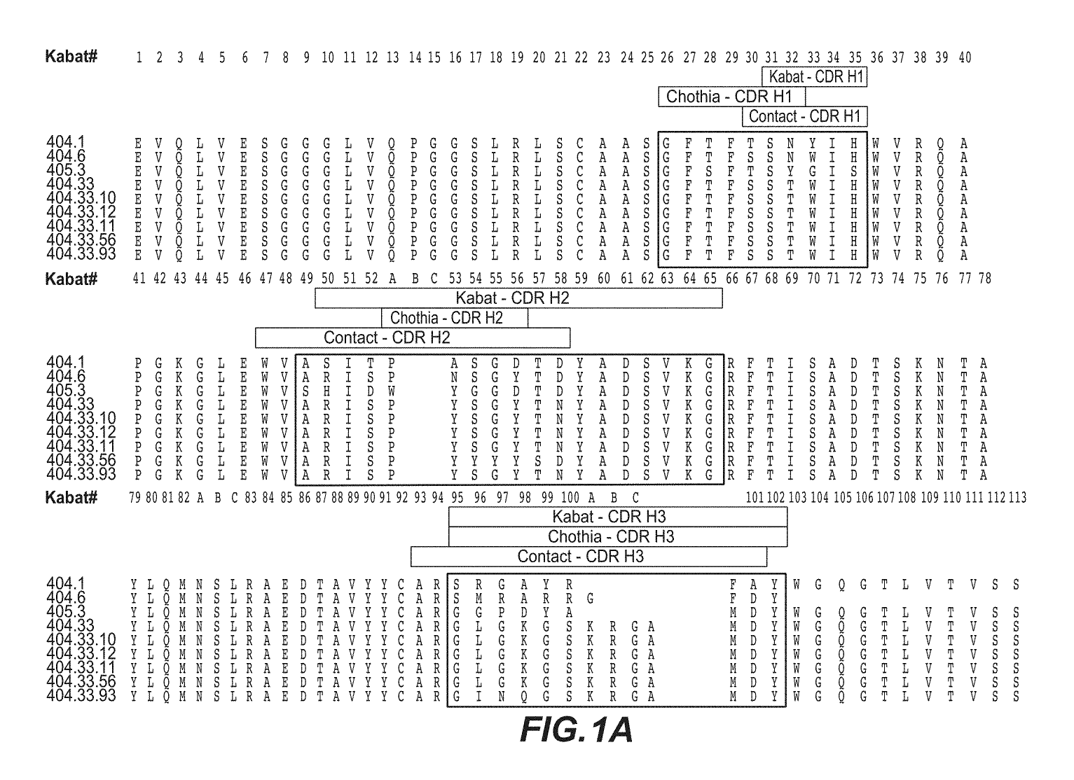

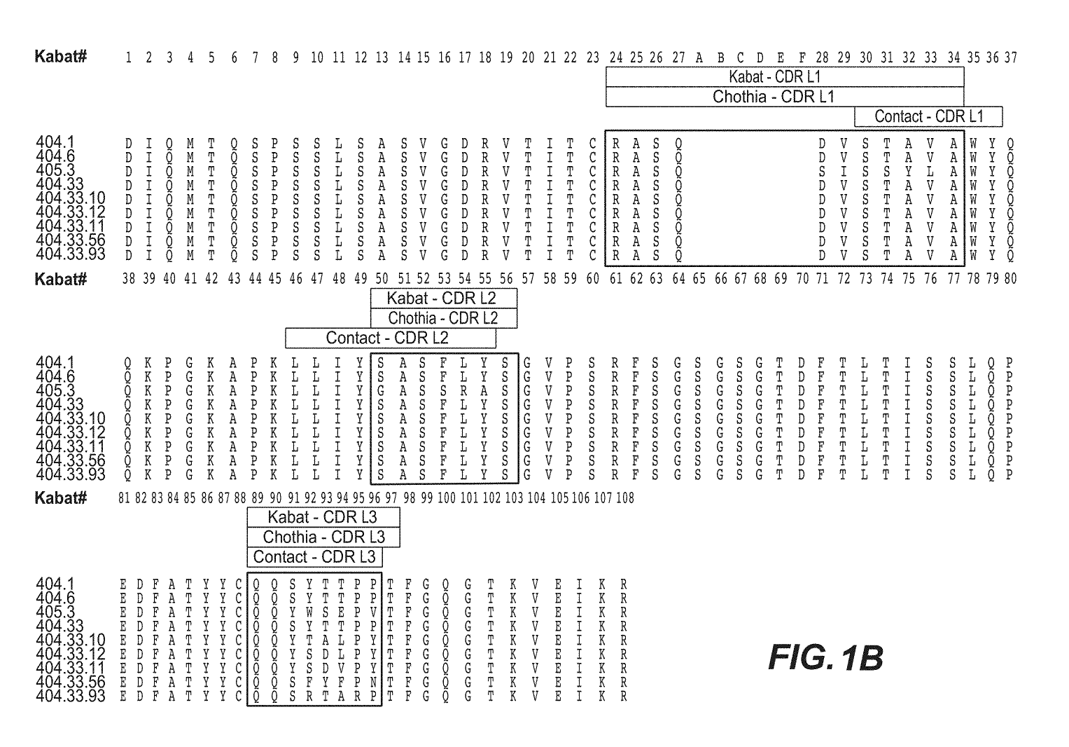

In one aspect, the invention provides an anti-IL-34 antibody comprising at least any one of one, two, three, four, or five, or six HVRs in any combination as shown in FIG. 1A and FIG. 1B. In some embodiments, the anti-IL-34 antibody comprises at least any one of one, two, three, four, or five, or six HVRs selected from (a) HVR-H1 comprising an amino acid sequence of STWIH (SEQ ID NO: 59); (b) HVR-H2 comprising an amino acid sequence RISPYYYYSDYADSVKG (SEQ ID NO: 52); (c) HVR-H3 comprising an amino acid sequence GLGKGSKRGAMDY (SEQ ID NO: 33); (d) HVR-L1 comprising an amino acid sequence RASQDVSTAVA (SEQ ID NO: 50); (e) HVR-L2 comprising an amino acid sequence SASFLYS (SEQ ID NO: 53); and (f) HVR-L3 comprising an amino acid sequence QQSFYFPNT (SEQ ID NO: 39). In some embodiments, the anti-IL-34 antibody comprises at least any one of one, two, three, four, or five, or six HVRs selected from (a) HVR-H1 comprising an amino acid sequence STWIH (SEQ ID NO: 59) or GFTFSST (SEQ ID NO: 30) or SSTWIH (SEQ ID NO: 57), (b) HVR-H2 comprising an amino acid sequence RISPYYYYSDYADSVKG (SEQ ID NO: 52) or PYYYY (SEQ ID NO: 37) or WVARISPYYYYSD (SEQ ID NO: 62); (c) HVR-H3 comprising an amino acid sequence GLGKGSKRGAMDY (SEQ ID NO: 33) or ARGLGKGSKRGAMD (SEQ ID NO: 28); (d) HVR-L1 comprising an amino acid sequence RASQDVSTAVA (SEQ ID NO: 50) or STAVAWY (SEQ ID NO: 58); (e) HVR-L2 comprising an amino acid sequence SASFLYS (SEQ ID NO: 53) or LLIYSASFLY (SEQ ID NO: 34); and (f) HVR-L3 comprising an amino acid sequence of QQSFYFPNT (SEQ ID NO: 39) or QQSFYFPN (SEQ ID NO: 38).

In some embodiments, the anti-IL-34 antibody comprises at least any one of one, two, three, four, or five, or six HVRs selected from (a) HVR-H1 comprising an amino acid sequence of STWIH (SEQ ID NO: 59); (b) HVR-H2 comprising an amino acid sequence RISPYSGYTNYADSVKG (SEQ ID NO: 51); (c) HVR-H3 comprising an amino acid sequence GLGKGSKRGAMDY (SEQ ID NO: 33); (d) HVR-L1 comprising an amino acid sequence of RASQDVSTAVA (SEQ ID NO: 50); (e) HVR-L2 comprising an amino acid sequence SASFLYS (SEQ ID NO: 53); and (f) HVR-L3 comprising an amino acid sequence QQYSDLPYT (SEQ ID NO: 45). In some embodiments, the anti-IL-34 antibody comprises at least any one of one, two, three, four, or five, or six HVRs selected from (a) HVR-H1 comprising an amino acid sequence STWIH (SEQ ID NO: 59) or GFTFSST (SEQ ID NO: 30) or SSTWIH (SEQ ID NO: 57)(b) HVR-H2 comprising an amino acid sequence RISPYSGYTNYADSVKG (SEQ ID NO: 51) or PYSGY (SEQ ID NO: 36) or WVARISPYSGYTN (SEQ ID NO: 61); (c) HVR-H3 comprising an amino acid sequence GLGKGSKRGAMDY (SEQ ID NO: 33) or ARGLGKGSKRGAMD (SEQ ID NO: 28); (d) HVR-L1 comprising an amino acid sequence of RASQDVSTAVA (SEQ ID NO: 50) or STAVAWY (SEQ ID NO: 58); (e) HVR-L2 comprising the amino acid sequence of SASFLYS (SEQ ID NO: 53) or LLIYSASFLY (SEQ ID NO: 34); and (f) HVR-L3 comprising an amino acid sequence QQYSDLPYT (SEQ ID NO: 45) or QQYSDLPY (SEQ ID NO: 44).

In some embodiments, the anti-IL-34 antibody comprises at least any one of one, two, three, four, five, or six HVRs selected from (a) a HVR-H1 comprising an amino acid sequence STWIH (SEQ ID NO: 59); (b) a HVR-H2 comprising an amino acid sequence RISPYYYYSDYADSVKG (SEQ ID NO: 52) or RISPYSGYTNYADSVKG (SEQ ID NO: 51); (c) a HVR-H3 comprising an amino acid sequence GLGKGSKRGAMDY (SEQ ID NO: 33) or GINQGSKRGAMDY (SEQ ID NO: 32); (d) a HVR-L1 comprising an amino acid sequence RASQDVSTAVA (SEQ ID NO: 50); (e) a HVR-L2 comprising an amino acid sequence SASFLYS (SEQ ID NO: 53); and (f) a HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41). In some embodiments, the anti-IL-34 antibody comprises (a) a HVR-H3 comprising an amino acid sequence GLGKGSKRGAMDY (SEQ ID NO: 33) or GINQGSKRGAMDY (SEQ ID NO: 32); (b) a HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41); and (c) a HVR-H2 comprising an amino acid sequence RISPYYYYSDYADSVKG (SEQ ID NO: 52) or RISPYSGYTNYADSVKG (SEQ ID NO: 51). In some embodiments, the anti-IL-34 antibody comprises at least any one of one, two, three, four, or five, or six HVRs selected from (a) HVR-H1 comprising an amino acid sequence STWIH (SEQ ID NO: 59) or GFTFSST (SEQ ID NO: 30) or SSTWIH (SEQ ID NO: 57); (b) HVR-H2 comprising an amino acid sequence RISPYYYYSDYADSVKG (SEQ ID NO: 52) or RISPYSGYTNYADSVKG (SEQ ID NO: 51) or PYYYY (SEQ ID NO: 37) or PYSGY (SEQ ID NO: 36) or WVARISPYYYYSD (SEQ ID NO: 62) or WVARISPYSGYTN (SEQ ID NO: 61); and (c) HVR-H3 comprising an amino acid sequence GLGKGSKRGAMDY (SEQ ID NO: 33) or GINQGSKRGAMDY (SEQ ID NO: 32) or ARGLGKGSKRGAMD (SEQ ID NO: 28) or ARGINQGSKRGAMD (SEQ ID NO: 27); (d) HVR-L1 comprising an amino acid sequence RASQDVSTAVA (SEQ ID NO: 50) or STAVAWY (SEQ ID NO: 58); (e) HVR-L2 comprising an amino acid sequence SASFLYS (SEQ ID NO: 53) or LLIYSASFLY (SEQ ID NO: 34); and (f) HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41) or QQSFYFPN (SEQ ID NO: 38) or QQSYTTPP (SEQ ID NO: 42) or QQYTALPY (SEQ ID NO: 48) or QQYSDLPY (SEQ ID NO: 44) or QQYSDVPY (SEQ ID NO: 46) or QQSRTARP (SEQ ID NO: 40).

In some embodiments, the anti-IL-34 antibody comprises at least any one of one, two, three, four, or five, or six HVRs selected from (a) a HVR-H1 comprising an amino acid sequence SNYIH (SEQ ID NO: 55); (b) a HVR-H2 comprising an amino acid sequence SITPASGDTDYADSVKG (SEQ ID NO: 54); (c) a HVR-H3 comprising an amino acid sequence SRGAYRFAY (SEQ ID NO: 56); (d) a HVR-L1 comprising an amino acid sequence RASQDVSTAVA (SEQ ID NO: 50); (e) a HVR-L2 comprising an amino acid sequence SASFLYS (SEQ ID NO: 53); and (f) a HVR-L3 comprising an amino acid sequence QQSYTTPPT (SEQ ID NO: 43). In some embodiments, the anti-IL-34 antibody comprises (a) a HVR-H3 comprising an amino acid sequence SRGAYRFAY (SEQ ID NO: 56); (b) a HVR-L3 comprising an amino acid sequence QQSYTTPPT (SEQ ID NO: 43); and (c) a HVR-H2 comprising an amino acid sequence SITPASGDTDYADSVKG (SEQ ID NO: 54). In some embodiments, the anti-IL-34 antibody comprises at least any one of one, two, three, four, or five, or six HVRs selected from (a) HVR-H1 comprising an amino acid sequence SNYIH (SEQ ID NO: 55) or GFTFTSN (SEQ ID NO: 31) or TSNYIH (SEQ ID NO: 60); (b) HVR-H2 comprising an amino acid sequence SITPASGDTDYADSVKG (SEQ ID NO: 54) or PASGD (SEQ ID NO: 35) or WVASITPASGDTD (SEQ ID NO: 63); (c) HVR-H3 comprising an amino acid sequence SRGAYRFAY (SEQ ID NO: 56) or ARSRGAYRFA (SEQ ID NO: 29); (d) HVR-L1 comprising an amino acid sequence RASQDVSTAVA (SEQ ID NO: 50) or STAVAWY (SEQ ID NO: 58); (e) HVR-L2 comprising an amino acid sequence SASFLYS (SEQ ID NO: 53) or LLIYSASFLY (SEQ ID NO: 34); and (f) HVR-L3 comprising an amino acid sequence QQSYTTPPT (SEQ ID NO: 43) or QQSYTTPP (SEQ ID NO: 42).

In one aspect, the invention provides an anti-IL-34 antibody comprising at least one, at least two, or all three VH HVR sequences selected from (a) HVR-H1 comprising an amino acid sequence STWIH (SEQ ID NO: 59); (b) HVR-H2 comprising an amino acid sequence RISPYYYYSDYADSVKG (SEQ ID NO: 52); (c) HVR-H3 comprising an amino acid sequence GLGKGSKRGAMDY (SEQ ID NO: 33). In some embodiments, the anti-IL-34 antibody comprises HVR-H3 comprising an amino acid sequence GLGKGSKRGAMDY (SEQ ID NO: 33). In some embodiments, the anti-IL-34 antibody comprises (a) HVR-H3 comprising an amino acid sequence GLGKGSKRGAMDY (SEQ ID NO: 33), and (b) a HVR-L3 comprising an amino acid sequence QQSFYFPNT (SEQ ID NO: 39). In some embodiments, the anti-IL-34 antibody comprises (a) HVR-H3 comprising an amino acid sequence GLGKGSKRGAMDY (SEQ ID NO: 33); (b) HVR-L3 comprising an amino acid sequence QQSFYFPNT (SEQ ID NO: 39); and (c) HVR-H2 comprising an amino acid sequence RISPYYYYSDYADSVKG (SEQ ID NO: 52). In some embodiments, the antibody comprises (a) HVR-H1 comprising an amino acid sequence STWIH (SEQ ID NO: 59); (b) HVR-H2 comprising an amino acid sequence RISPYYYYSDYADSVKG (SEQ ID NO: 52); and (c) HVR-H3 comprising an amino acid sequence GLGKGSKRGAMDY (SEQ ID NO: 33).

In another aspect, the invention provides an anti-IL-34 antibody comprising at least one, at least two, or all three VL HVR sequences selected from (a) HVR-L1 comprising an amino acid sequence RASQDVSTAVA (SEQ ID NO: 50); (b) HVR-L2 comprising an amino acid sequence SASFLYS (SEQ ID NO: 53); and (c) HVR-L3 comprising an amino acid sequence QQSFYFPNT (SEQ ID NO: 39). In some embodiments, the antibody comprises (a) HVR-L1 comprising an amino acid sequence RASQDVSTAVA (SEQ ID NO: 50); (b) HVR-L2 comprising an amino acid sequence SASFLYS (SEQ ID NO: 53); and (c) HVR-L3 comprising an amino acid sequence QQSFYFPNT (SEQ ID NO: 39).

In another aspect, an anti-IL-34 antibody of the invention comprises (a) a VH domain comprising at least one, at least two, or all three VH HVR sequences selected from (i) HVR-H1 comprising an amino acid sequence STWIH (SEQ ID NO: 59), (ii) HVR-H2 comprising an amino acid sequence RISPYYYYSDYADSVKG (SEQ ID NO: 52), and (iii) HVR-H3 comprising an amino acid sequence GLGKGSKRGAMDY (SEQ ID NO: 33); and (b) a VL domain comprising at least one, at least two, or all three VL HVR sequences selected from (i) HVR-L1 comprising an amino acid sequence RASQDVSTAVA (SEQ ID NO: 50), (ii) HVR-L2 comprising an amino acid sequence SASFLYS (SEQ ID NO: 53), and (iii) HVR-L3 comprising an amino acid sequence QQSFYFPNT (SEQ ID NO: 39).

In another aspect, the invention provides an anti-IL-34 antibody comprising (a) HVR-H1 comprising an amino acid sequence of STWIH (SEQ ID NO: 59); (b) HVR-H2 comprising an amino acid sequence RISPYYYYSDYADSVKG (SEQ ID NO: 52); (c) HVR-H3 comprising an amino acid sequence GLGKGSKRGAMDY (SEQ ID NO: 33); (d) HVR-L1 comprising an amino acid sequence RASQDVSTAVA (SEQ ID NO: 50); (e) HVR-L2 comprising an amino acid sequence SASFLYS (SEQ ID NO: 53); and (f) HVR-L3 comprising an amino acid sequence QQSFYFPNT (SEQ ID NO: 39).

In one aspect, the invention provides an anti-IL-34 antibody comprising at least one, at least two, or all three VH HVR sequences selected from (a) HVR-H1 comprising an amino acid sequence STWIH (SEQ ID NO: 59); (b) HVR-H2 comprising an amino acid sequence RISPYSGYTNYADSVKG (SEQ ID NO: 51); (c) HVR-H3 comprising an amino acid sequence GLGKGSKRGAMDY (SEQ ID NO: 33). In some embodiments, the anti-IL-34 antibody comprises HVR-H3 comprising an amino acid sequence GLGKGSKRGAMDY (SEQ ID NO: 33). In some embodiments, the anti-IL-34 antibody comprises (a) HVR-H3 comprising an amino acid sequence GLGKGSKRGAMDY (SEQ ID NO: 33), and (b) a HVR-L3 comprising an amino acid sequence QQYSDLPYT (SEQ ID NO: 45). In some embodiments, the anti-IL-34 antibody comprises (a) HVR-H3 comprising an amino acid sequence GLGKGSKRGAMDY (SEQ ID NO: 33); (b) HVR-L3 comprising an amino acid sequence QQYSDLPYT (SEQ ID NO: 45); and (c) HVR-H2 comprising an amino acid sequence RISPYSGYTNYADSVKG (SEQ ID NO: 51). In some embodiments, the antibody comprises (a) HVR-H1 comprising an amino acid sequence STWIH (SEQ ID NO: 59); (b) HVR-H2 comprising an amino acid sequence RISPYSGYTNYADSVKG (SEQ ID NO: 51); and (c) HVR-H3 comprising an amino acid sequence GLGKGSKRGAMDY (SEQ ID NO: 33).

In another aspect, the invention provides an anti-IL-34 antibody comprising at least one, at least two, or all three VL HVR sequences selected from (a) HVR-L1 comprising an amino acid sequence of RASQDVSTAVA (SEQ ID NO: 50); (b) HVR-L2 comprising an amino acid sequence SASFLYS (SEQ ID NO: 53); and (c) HVR-L3 comprising an amino acid sequence QQYSDLPYT (SEQ ID NO: 45). In some embodiments, the antibody comprises (a) HVR-L1 comprising an amino acid sequence RASQDVSTAVA (SEQ ID NO: 50); (b) HVR-L2 comprising an amino acid sequence SASFLYS (SEQ ID NO: 53); and (c) HVR-L3 comprising an amino acid sequence QQYSDLPYT (SEQ ID NO: 45).

In another aspect, an anti-IL-34 antibody of the invention comprises (a) a VH domain comprising at least one, at least two, or all three VH HVR sequences selected from (i) HVR-H1 comprising an amino acid sequence STWIH (SEQ ID NO: 59), (ii) HVR-H2 comprising an amino acid sequence RISPYSGYTNYADSVKG (SEQ ID NO: 51), (iii) HVR-H3 comprising an amino acid sequence GLGKGSKRGAMDY (SEQ ID NO: 33); and (b) a VL domain comprising at least one, at least two, or all three VL HVR sequences selected from (i) HVR-L1 comprising an amino acid sequence of RASQDVSTAVA (SEQ ID NO: 50); (ii) HVR-L2 comprising an amino acid sequence SASFLYS (SEQ ID NO: 53); and (iii) HVR-L3 comprising an amino acid sequence QQYSDLPYT (SEQ ID NO: 45).

In another aspect, the invention provides an anti-IL-34 antibody comprising (a) HVR-H1 comprising an amino acid sequence STWIH (SEQ ID NO: 59); (b) HVR-H2 comprising an amino acid sequence RISPYSGYTNYADSVKG (SEQ ID NO: 51); (c) HVR-H3 comprising an amino acid sequence GLGKGSKRGAMDY (SEQ ID NO: 33); (d) HVR-L1 comprising an amino acid sequence of RASQDVSTAVA (SEQ ID NO: 50); (e) HVR-L2 comprising an amino acid sequence SASFLYS (SEQ ID NO: 53); and (f) HVR-L3 comprising an amino acid sequence QQYSDLPYT (SEQ ID NO: 45).

In another aspect, the invention provides an anti-IL-34 antibody comprising (a) HVR-H1 comprising an amino acid sequence SNWIH (SEQ ID NO:70), (b) HVR-H2 comprising an amino acid sequence RISPNSGYTDYADSVKG (SEQ ID NO: 71); (c) HVR-H3 comprising an amino acid sequence SMRARRGFDY (SEQ ID NO: 72); (d) HVR-L1 comprising an amino acid sequence of RASQDVSTAVA (SEQ ID NO: 50); (e) HVR-L2 comprising an amino acid sequence SASFLYS (SEQ ID NO: 53); and (f) HVR-L3 comprising an amino acid sequence QQSYTTPPT (SEQ ID NO: 43).

In another aspect, the invention provides an anti-IL-34 antibody derived from an anti-IL-34 antibody exemplified herein.

In some embodiments, the anti-IL-34 antibody comprises any one or any combination of two, three, four, five, or six of the following HVRs:

HVR-H1: SX₁X₂IH, wherein X₁ is N or T, and X₂ is Y or W (SEQ ID NO: 64);

HVR-H2: X₁IX₂PX₃X₄X₅X₆X₇X₈YADSVKG, wherein X₁ is S or R; and X₂ is T or S; X₃ is A or Y; X₄ is S or Y; X₅ is G or Y; X₆ is D or Y; X₇ is T or S; and X₈ is D or N (SEQ ID NO: 65);

HVR-H3: SRGAYRFAY (SEQ ID NO: 56), or GX₁X₂X₃GSKRGAMDY, wherein X₁ is L or I; X₂ is G or N; X₃ is K or Q (SEQ ID NO: 66);

HVR-L1: RASQDVSTAVA (SEQ ID NO: 50);

HVR-L2: SASFLYS (SEQ ID NO: 53);

HVR-L3: QQ X₁IX₂PX₃X₄X₅X₆T, wherein the X₁ is S or Y; and X₂ is Y, T, S, F, or R; X₃ is T, A, D, or Y; X₄ is T, L, V, F, or A; X₅ is P or R; X₆ is P, Y, or N (SEQ ID NO: 67).

In some embodiments, one or more amino acid residues in HVRs may be substituted. In some embodiments, the substitutions are conservative substitutions, as provided herein.

In any of the above embodiments, an anti-IL-34 antibody is humanized. In some embodiments, an anti-IL-34 antibody comprises HVRs as in any of the above embodiments, and further comprises an acceptor human framework, e.g., a human immunoglobulin framework or a human consensus framework. In another embodiment, an anti-IL-34 antibody comprises HVRs as in any of the above embodiments, and further comprises a VH comprising an FR1 sequence of SEQ ID NO:17, an FR2 sequence of SEQ ID NO:18, an FR3 sequence of SEQ ID NO:19, a FR4 sequence of SEQ ID NO:20 and/or a VL comprising an FR1 sequence of SEQ ID NO:21, an FR2 sequence of SEQ ID NO:22, an FR3 sequence of SEQ ID NO:23, a FR4 sequence of SEQ ID NO:24.

In another aspect, an anti-IL-34 antibody comprises a heavy chain variable domain (VH) sequence having at least any one of 90%, 91%, 92%, 93%, 94%, 95%, 96%, 97%, 98%, or 99%, or 100% sequence identity to the amino acid sequence of SEQ ID NO:3 (VH amino acid sequence of antibody 404.33.56 shown in FIG. 1A). In some embodiments, a VH sequence having at least any one of 90%, 91%, 92%, 93%, 94%, 95%, 96%, 97%, or 98%, or 99% identity contains substitutions (e.g., conservative substitutions), insertions, or deletions relative to the reference sequence, but an anti-IL-34 antibody comprising that sequence retains the ability to bind to IL-34. In some embodiments, a total of 1 to 10 amino acids have been substituted, inserted and/or deleted in SEQ ID NO:3. In some embodiments, substitutions, insertions, or deletions occur in regions outside the HVRs (i.e., in the FRs). Optionally, the anti-IL-34 antibody comprises the VH sequence in SEQ ID NO:3, including post-translational modifications of that sequence. In a particular embodiment, the VH comprises one, two or three HVRs selected from: (a) HVR-H1 comprising an amino acid sequence STWIH (SEQ ID NO: 59); (b) HVR-H2 comprising an amino acid sequence RISPYYYYSDYADSVKG (SEQ ID NO: 52); (c) HVR-H3 comprising an amino acid sequence GLGKGSKRGAMDY (SEQ ID NO: 33).

In another aspect, an anti-IL-34 antibody is provided, wherein the antibody comprises a light chain variable domain (VL) having at least any one of 90%, 91%, 92%, 93%, 94%, 95%, 96%, 97%, 98%, or 99%, or 100% sequence identity to the amino acid sequence of SEQ ID NO:4 (VL amino acid sequence of antibody 404.33.56 shown in FIG. 1B). In some embodiments, a VL sequence having at least any one of 90%, 91%, 92%, 93%, 94%, 95%, 96%, 97%, or 98%, or 99% identity contains substitutions (e.g., conservative substitutions), insertions, or deletions relative to the reference sequence, but an anti-IL-34 antibody comprising that sequence retains the ability to bind to IL-34. In some embodiments, a total of 1 to 10 amino acids have been substituted, inserted and/or deleted in SEQ ID NO:4. In some embodiments, the substitutions, insertions, or deletions occur in regions outside the HVRs (i.e., in the FRs). Optionally, the anti-IL-34 antibody comprises the VL sequence in SEQ ID NO:4, including post-translational modifications of that sequence. In a particular embodiment, the VL comprises one, two or three HVRs selected from (a) HVR-L1 comprising an amino acid sequence RASQDVSTAVA (SEQ ID NO: 50); (b) HVR-L2 comprising an amino acid sequence SASFLYS (SEQ ID NO: 53); and (c) HVR-L3 comprising an amino acid sequence QQSFYFPNT (SEQ ID NO: 39).

In another aspect, an anti-IL-34 antibody comprises a heavy chain variable domain (VH) sequence having at least any one of 90%, 91%, 92%, 93%, 94%, 95%, 96%, 97%, 98%, or 99%, or 100% sequence identity to the amino acid sequence of SEQ ID NO:11 (VH amino acid sequence of antibody 404.33.12 shown in FIG. 1A). In some embodiments, a VH sequence having at least any one of 90%, 91%, 92%, 93%, 94%, 95%, 96%, 97%, or 98%, or 99% identity contains substitutions (e.g., conservative substitutions), insertions, or deletions relative to the reference sequence, but an anti-IL-34 antibody comprising that sequence retains the ability to bind to IL-34. In some embodiments, a total of 1 to 10 amino acids have been substituted, inserted and/or deleted in SEQ ID NO:11. In some embodiments, substitutions, insertions, or deletions occur in regions outside the HVRs (i.e., in the FRs). Optionally, the anti-IL-34 antibody comprises the VH sequence in SEQ ID NO:11, including post-translational modifications of that sequence. In a particular embodiment, the VH comprises one, two or three HVRs selected from: (a) HVR-H1 comprising an amino acid sequence of STWIH (SEQ ID NO: 59); (b) HVR-H2 comprising an amino acid sequence RISPYSGYTNYADSVKG (SEQ ID NO: 51); (c) HVR-H3 comprising an amino acid sequence GLGKGSKRGAMDY (SEQ ID NO: 33).

In another aspect, an anti-IL-34 antibody is provided, wherein the antibody comprises a light chain variable domain (VL) having at least any one of 90%, 91%, 92%, 93%, 94%, 95%, 96%, 97%, 98%, or 99%, or 100% sequence identity to the amino acid sequence of SEQ ID NO:12 (VL amino acid sequence of antibody 404.33.12 shown in FIG. 1B). In some embodiments, a VL sequence having at least any one of 90%, 91%, 92%, 93%, 94%, 95%, 96%, 97%, or 98%, or 99% identity contains substitutions (e.g., conservative substitutions), insertions, or deletions relative to the reference sequence, but an anti-IL-34 antibody comprising that sequence retains the ability to bind to IL-34. In some embodiments, a total of 1 to 10 amino acids have been substituted, inserted and/or deleted in SEQ ID NO:12. In some embodiments, the substitutions, insertions, or deletions occur in regions outside the HVRs (i.e., in the FRs). Optionally, the anti-IL-34 antibody comprises the VL sequence in SEQ ID NO:12, including post-translational modifications of that sequence. In a particular embodiment, the VL comprises one, two or three HVRs selected from (a) HVR-L1 comprising an amino acid sequence of RASQDVSTAVA (SEQ ID NO: 50); (b) HVR-L2 comprising an amino acid sequence SASFLYS (SEQ ID NO: 53); and (c) HVR-L3 comprising an amino acid sequence QQYSDLPYT (SEQ ID NO: 45).

In another aspect, an anti-IL-34 antibody is provided, wherein the antibody comprises a VH as in any of the embodiments provided above, and a VL as in any of the embodiments provided above. In some embodiments, the antibody comprises the VH and VL sequences in SEQ ID NO:3 and SEQ ID NO:4, respectively, including post-translational modifications of those sequences. In some embodiments, the antibody comprises the VH and VL sequences in SEQ ID NO: 11 and SEQ ID NO:12, respectively, including post-translational modifications of those sequences. In some embodiments, the antibody comprises the VH and VL sequences in SEQ ID NO:5 and SEQ ID NO:6, respectively, including post-translational modifications of those sequences. In some embodiments, the antibody comprises the VH and VL sequences in SEQ ID NO: 7 and SEQ ID NO:8, respectively, including post-translational modifications of those sequences. In some embodiments, the antibody comprises the VH and VL sequences in SEQ ID NO: 9 and SEQ ID NO:10, respectively, including post-translational modifications of those sequences. In some embodiments, the antibody comprises the VH and VL sequences in SEQ ID NO:13 and SEQ ID NO:14, respectively, including post-translational modifications of those sequences. In some embodiments, the antibody comprises the VH and VL sequences in SEQ ID NO:15 and SEQ ID NO:16, respectively, including post-translational modifications of those sequences. In some embodiments, the antibody comprises the VH and VL sequences in SEQ ID NO:68 and SEQ ID NO:69, respectively, including post-translational modifications of those sequences.

In a further aspect, the invention provides an antibody that binds to the same epitope as an anti-IL-34 antibody provided herein. For example, in some embodiments, an antibody is provided that binds to the same epitope as an anti-IL-34 antibody selected from the of an anti-IL-34 antibody comprising a VH sequence of SEQ ID NO:3 and a VL sequence of SEQ ID NO:4, an anti-IL-34 antibody comprising a VH sequence of SEQ ID NO:11 and a VL sequence of SEQ ID NO:12, an anti-IL-34 antibody comprising a VH sequence of SEQ ID NO:5 and a VL sequence of SEQ ID NO:6, an anti-IL-34 antibody comprising a VH sequence of SEQ ID NO:7 and a VL sequence of SEQ ID NO:8, an anti-IL-34 antibody comprising a VH sequence of SEQ ID NO:9 and a VL sequence of SEQ ID NO:10, an anti-IL-34 antibody comprising a VH sequence of SEQ ID NO:13 and a VL sequence of SEQ ID NO:14, or an anti-IL-34 antibody comprising a VH sequence of SEQ ID NO:15 and a VL sequence of SEQ ID NO:16. In some embodiments, the anti-IL-34 antibody binds to the same epitope as an anti-IL-34 antibody comprising a VH sequence of SEQ ID NO:3 and a VL sequence of SEQ ID NO:4. In some embodiments, the anti-IL-34 antibody binds to the same epitope as an anti-IL-34 antibody comprising a VH sequence of SEQ ID NO:11 and a VL sequence of SEQ ID NO:12. In some embodiments, the epitope is a conformational epitope In some embodiments, the anti-IL-34 antibody binds to the same epitope as an anti-IL-34 antibody comprising a VH sequence of SEQ ID NO:68 and a VL sequence of SEQ ID NO:69. In some embodiments, the epitope is a conformational epitope. In some embodiments, the epitope is a linear epitope.

In a further aspect of the invention, an anti-IL-34 antibody according to any of the above embodiments is a monoclonal antibody, including a chimeric, humanized or human antibody. In some embodiments, an anti-IL-34 antibody is an antibody fragment, e.g., a Fv, Fab, Fab′, scFv, diabody, or F(ab′)₂ fragment. In another embodiment, the antibody is a full length antibody, e.g., an intact IgG1 or IgG4 antibody or other antibody class or isotype as defined herein.

In a further aspect, an anti-IL-34 antibody according to any of the above embodiments may incorporate any of the features, singly or in combination, as described in Sections 1-7 below:

### Anti-CSF-1R Inhibitors

In another aspect, the invention provides a method of treating a neurological disease in an individual, treating an individual exhibiting one or more symptoms of a neurological disease, or reducing the density of microglia in the brain of an individual by administering to the individual an effective amount of an anti-IL-34 antibody and an effective amount of a CSF-1R inhibitor. In some embodiments, the CSF-1R inhibitor is a small molecule inhibitor, including without limitation, GW2580. In some embodiments, the CSF-1R inhibitor is an isolated antibody that binds to CSF-1R (e.g., human CSF-1R). In some embodiments, provided herein is an anti-CSF-1R antibody, which binds to an epitope comprising at least any one of one, two, three, four, or five, or six of amino acid residues Arg144, Gln248, Gln249, Ser250, Phe252, and Asn254 of human CSF-1R. In one aspect, provided herein is an anti-CSF-1R antibody, which binds to an epitope comprising amino acid residue Arg144 of human CSF-1R. In one aspect, provided herein is an anti-CSF-1R antibody, which binds to an epitope comprising at least any one of one, two, or three, or four of amino acid residues Arg144, Arg142, Arg146, and Arg250 of human CSF-1R. The anti-CSF-1R antibody of any of the aspects above may bind to an epitope further comprising at least one, or two of amino acid residues Ser172 and Arg192 of human CSF-1R. The anti-CSF-1R antibody of any of the aspects above may bind to an epitope further comprising at least any one of one, two, three, four, or five, or six of amino acid residues Arg146, Met149, Arg150, Phe169, Ile170, and Gln173 of human CSF-1R. In some embodiments, the anti-CSF-1R antibody binds to amino acids within positions 142-150 and 169-172 of CSF-1R. As used herein, the residue position herein corresponds to the residue position in SEQ ID NO:2. In some embodiments, the anti-CSF-1R antibody inhibits binding between human IL-34 and/or human CSF-1 to human CSF-1R.

In another aspect, provided herein is an anti-CSF-1R antibody, which binds to an epitope comprising at least any one of one, two, three, four, or five, or six of amino acid residues Arg144, Gln248, Gln249, Ser250, Phe252, and Asn 254 of human CSF-1R. In one aspect, provided herein is an anti-CSF-1R antibody, which binds to an epitope comprising at least any one of one, two, three, or four, or five of amino acid residues Gln248, Gln249, Ser250, Phe252, and Asn254 of human CSF-1R. In one aspect, provided herein is an anti-CSF-1R antibody, which binds to an epitope comprising at least any one of one, two, three, four, or five, or six of amino acid residues Gln248, Gln249, Ser250, Phe252, Asn254, and Tyr257 of human CSF-1R. The anti-CSF-1R antibody of any of the aspects above may bind to an epitope further comprising at least one, at least two, or three of amino acid residues Pro247, Gln258, and Lys259 of human CSF-1R. The anti-CSF-1R antibody of any of the aspects above may bind to an epitope further comprising at least one, at least two, or three of amino acid residues Val231, Asp251, and Tyr257 of human CSF-1R. In some embodiments, the anti-CSF-1R antibody binds to amino acids within positions 231, 248-252 and 254 of CSF-1R. As used herein, the residue position herein corresponds to the residue position in SEQ ID NO:2. In some embodiments, the anti-CSF-1R antibody inhibits binding between human IL-34 and/or human CSF-1 to human CSF-1R.

In a further aspect of the invention, an anti-CSF-1R antibody according to any of the above embodiments is a monoclonal antibody, including a chimeric, humanized or human antibody. In some embodiments, anti-CSF-1R antibody is an antibody fragment, e.g., a Fv, Fab, Fab′, scFv, diabody, or F(ab′)₂ fragment. In another embodiment, the antibody is a full length antibody, e.g., an intact IgG1 or IgG4 antibody or other antibody class or isotype as defined herein.

In a further aspect, an anti-IL-34 antibody or anti-CSF-1R antibody according to any of the above embodiments may incorporate any of the features, singly or in combination, as described in Sections 1-7 below:

1. Antibody Affinity

In some embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20®) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to another embodiment, the Kd is measured using a BLI assay, for example, as described herein.

2. Antibody Fragments

In some embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (Domantis, Inc., Waltham, Mass.; see, e.g., U.S. Pat. No. 6,248,516 B1).

In some embodiments, an antibody fragment is a monovalent antibody that has an in vivo half-life substantially similar to an intact antibody. For example, such an antibody fragment may comprise one antigen binding arm linked to an Fc sequence capable of conferring in vivo stability to the fragment. In one embodiment, an antibody of the invention is a one-armed antibody as described in WO2005/063816. In one embodiment, the one-armed antibody comprises Fc mutations constituting “knobs” and “holes” as described in WO2005/063816.

The antibody fragment may also be a “linear antibody”, e.g., as described in U.S. Pat. No. 5,641,870. Such linear antibody fragments may be monospecific or bispecific.

Antibody fragments can be made by various techniques, including but not limited to proteolytic digestion of an intact antibody as well as production by recombinant host cells (e.g., E. coli or phage), as described herein.

3. Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall' Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA, 89:4285 (1992); and Presta et al., J. Immunol., 151:2623 (1993)); human mature (somatically mutated) framework regions or human germline framework regions (see, e.g., Almagro and Frans son, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In some embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J., Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the present disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

### Bispecific Antibodies

Bispecific antibodies are monoclonal antibodies that have binding specificities for two different antigens. In some embodiments, bispecific antibodies are human or humanized antibodies. In some embodiments, one of the binding specificities is for IL-34 (e.g., human IL-34) and the other is for any other antigen. In some embodiments, bispecific antibodies may bind to two different epitopes of IL-34 (e.g., human IL-34). In some embodiments, bispecific antibodies comprise a first binding specificity to IL-34 (e.g., human IL-34) and a second binding specificity to CSF-1 (e.g., human CSF-1). In some embodiments, bispecific antibodies bind to the same epitope on IL-34 as any of the anti-IL-34 antibodies described herein. In some embodiments, bispecific antibodies comprise at least any one of one, two, three, four, or five or six HVRs of any one of the anti-IL-34 antibodies described herein. Bispecific antibodies can be prepared as full length antibodies or antibody fragments (e.g., F(ab′)₂ bispecific antibodies).

Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305: 537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBOJ., 10: 3655 (1991).

According to a different approach, antibody variable domains with the desired binding specificities (antibody-antigen combining sites) are fused to immunoglobulin constant domain sequences. The fusion, for example, is with an immunoglobulin heavy chain constant domain, comprising at least part of the hinge, CH2, and CH3 regions. In some embodiments, the first heavy-chain constant region (CH1), containing the site necessary for light chain binding, is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach, the interface between a pair of antibody molecules can be engineered to maximize the percentage of heterodimers which are recovered from recombinant cell culture. The interface comprises at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/00373, and EP 03089). Heteroconjugate antibodies may be made using any convenient cross-linking method. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)2′ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

According to one embodiment, one polypeptide comprising an antigen binding domain of this invention comprises a heterodimerization domain. As used herein, “heteromultimerization domain” refers to alterations or additions to a biological molecule so as to promote heteromultimer formation and hinder homomultimer formation. Any heterodimerization domain having a strong preference for forming heterodimers over homodimers is within the scope of the invention. Illustrative examples include but are not limited to, for example, US Patent Application 20030078385 (Arathoon et al.; describing knob-into-holes); WO2007147901 (Kjergaard et al.; describing ionic interactions); WO 2009089004 (Kannan et al.; describing electrostatic steering effects); WO2011/034605 (Christensen et al.; describing coiled coils). See also, for example, Pack, P. & Plueckthun, A., Biochemistry 31, 1579-1584 (1992) describing leucine zipper or Pack et al., Bio/Technology 11, 1271-1277 (1993) describing the helix-turn-helix motif. The phrase “heteromultimerization domain” and “heterodimerization domain” are used interchangeably herein.

The term “knob-into-hole” or “KnH” technology as mentioned herein refers to the technology directing the pairing of two polypeptides together in vitro or in vivo by introducing a pertuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (e.g., US2007/0178552, WO 96/027011, WO 98/050431 and Zhu et al. (1997) Protein Science 6:781-788).

Further techniques for making multispecific, e.g., bispecific, antibodies include, but are not limited to, “knob-in-hole” engineering (see, e.g., U.S. Pat. No. 5,731,168), engineering using electrostatic steering effects for making antibody Fc-heterodimeric molecules (WO 2009/089004A1).

Antibodies with more than two valencies are contemplated. For example, trispecific antibodies can be prepared. Tutt et al., J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to IL-34 as well as another, different antigen (e.g., CSF-1) (see, US2008/0069820, for example).

7. Antibody Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In some embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b) Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., U.S. Patent Publication Nos. US 2003/0157108 (Presta, L.); U.S. 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); U.S. Pat Appl No US 2003/0157108 A1, Presta, L; and WO 2004/056312 A1, Adams et al., Acta crystallographica Section D, Biological crystallography 66: 213-221 (2010), especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In some embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al., Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al., J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan et al., Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In some embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

e) Antibody Derivatives

In some embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In some embodiments, isolated nucleic acid encoding an anti-IL-34 antibody, a bispecific anti-IL-34/CSF-1 antibody or an anti-CSF-1R antibody described herein is provided. Such nucleic acid may encode an amino acid sequence comprising the VL and/or an amino acid sequence comprising the VH of the antibody (e.g., the light and/or heavy chains of the antibody). In some embodiments, one or more vectors (e.g., expression vectors) comprising such nucleic acid are provided. In some embodiments, a host cell comprising such nucleic acid is provided. In some embodiments, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g., a Chinese Hamster Ovary (CHO) cell or lymphoid cell (e.g., Y0, NS0, Sp20 cell). In some embodiments, a method of making an anti-IL-34 antibody, a bispecific anti-IL-34/CSF-1 antibody or an anti-CSF-1R antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-IL-34 antibody, a bispecific anti-IL-34/CSF-1 antibody or an anti-CSF-1R antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

C. Assays

Anti-IL-34 antibodies, bispecific anti-IL-34/CSF-1 antibodies and anti-CSF-1R antibodies provided herein may be identified, screened for, or characterized for their physical/chemical properties and/or biological activities by various assays known in the art.

1. Binding Assays and Other Assays

In one aspect, an antibody of the present disclosure is tested for its antigen binding activity, e.g., by known methods such as ELISA, Western blot, etc.

In another aspect, competition assays may be used to identify an anti-IL-34 antibody or a bispecific anti-IL-34/CSF-1 antibody that competes with, for example, an anti-IL-34 antibody described herein. For example, antibodies that compete with an anti-IL-34 antibody comprising aVH sequence of SEQ ID NO:5 and a VL sequence of SEQ ID NO:6 for binding to IL-34. In some embodiments, such a competing antibody binds to the same epitope (e.g., a linear or a conformational epitope) that is bound by, for example, an anti-IL-34 antibody comprising aVH sequence of SEQ ID NO:5 and a VL sequence of SEQ ID NO:6. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized IL-34 is incubated in a solution comprising a first labeled antibody that binds to IL-34 (e.g., an anti-IL-34 antibody comprising aVH sequence of SEQ ID NO:5 and a VL sequence of SEQ ID NO:6) and a second unlabeled antibody that is being tested for its ability to compete with the first antibody for binding to IL-34. The second antibody may be present in a hybridoma supernatant. As a control, immobilized IL-34 is incubated in a solution comprising the first labeled antibody but not the second unlabeled antibody. After incubation under conditions permissive for binding of the first antibody to IL-34, excess unbound antibody is removed, and the amount of label associated with immobilized IL-34 is measured. If the amount of label associated with immobilized IL-34 is substantially reduced in the test sample relative to the control sample, then that indicates that the second antibody is competing with the first antibody for binding to IL-34. See Harlow and Lane (1988) Antibodies: A Laboratory Manual ch. 14 (Cold Spring Harbor Laboratory, Cold Spring Harbor, N.Y.).

In one aspect, assays are provided for identifying anti-IL-34 antibodies, bispecific anti-IL-34/CSF-1 antibodies or anti-CSF1R antibodies having biological activities. Biological activity may include, e.g., inhibition of proliferation of human peripheral blood mononuclear cells (PBMCs), inhibition of binding of IL-34 to CSF-1R, or inhibition of binding of CSF-1 to CSF-1R. Antibodies having such biological activity in vivo and/or in vitro are also provided.

In some embodiments, an antibody of the present disclosure is tested for such biological activity. For example, the neutralizing activity of an anti-IL-34 antibody, a bispecific anti-IL-34/CSF-1 antibody or anti-CSF-1R antibody can be measured using a cell proliferation assay by CellTiter-Glo. hIL-34 or mIL-34 is combined with serial dilutions of anti-IL-34 mAbs, bispecific anti-IL-34/CSF-1 antibodies or anti-CSF1 antibodies before adding onto cells, such as peripheral blood mononuclear cells (PBMCs). The antibody inhibition activity is obtained by measuring RLU after incubating the plates at 37° C. for 72 hours. The Half Maximal Inhibitory Concentration (IC50), defined as the concentration of antibody required to yield half maximal inhibition of IL-34 activity on cells, when IL-34 is present at a concentration to elicit 70-80% proliferation response, can be calculated with KaleidaGraph.

Inhibition of binding of IL-34 or CSF-1 to CSF-1R by an antibody provided herein may be tested in ELISA assays using immobilized IL-34 or CSF-1 and soluble CSF-1R in the presence of serial dilution of the antibody, e.g., an anti-IL-34 antibody, bispecific IL-34/CSF-1 antibody or anti-CSF-1 antibody.

D. Pharmaceutical Compositions

Pharmaceutical compositions of the present disclosure may contain an anti-IL-34 antibody and may further contain additional agents such as a bispecific anti-IL-34/CSF-1 antibody, an inhibitor of CSF-1R, and/or an anti-CSF-1 antibody as described herein. Pharmaceutical compositions are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO2006/044908, the latter composition including a histidine-acetate buffer.

The pharmaceutical composition herein may also contain more than one active ingredients as necessary for the particular indication being treated, preferably those with complementary activities that do not adversely affect each other. For example, it may be desirable to further provide an inhibitor of CSF-1R in addition to an anti-IL-34 antibody. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules.

The compositions to be used for in vivo administration are generally sterile. Sterility may be readily accomplished, e.g., by filtration through sterile filtration membranes.

E. Therapeutic Methods and Compositions

Further aspects of the present disclosure provide methods for treating a neurological disease in an individual comprising administering to the individual an effective amount of an anti-IL-34 antibody, treating an individual exhibiting one or more symptoms of a neurological disease comprising administering to the individual an effective amount of an anti-IL-34 antibody, and reducing the density of microglia in the brain of an individual comprising administering an effective amount of an anti-IL-34 antibody. In some embodiments the methods further include administering to the individual an effective amount of a CSF-1R inhibitor. In some embodiments the methods further include administering to the individual an effective amount of an anti-CSF-1 antibody. In some embodiments the anti-CSF-1 antibody inhibits binding of human CSF-1 to human CSF-1R. In certain embodiments, the individual is a mammal. In preferred embodiments, the individual is a human. Methods of treatment include, without limitation, preventing a disease or its symptoms, reducing the occurrence or recurrence of a disease, slowing the progress of a disease, relieving symptoms of a disease, diminishing any direct or indirect pathological consequences of the disease, increasing the life expectancy of an individual with a disease, ameliorating or palliating the disease state, improving prognosis, or curing a disease. In some embodiments of methods for treating a neurological disease in an individual, the density of microglia in the brain of the individual is reduced. In some embodiments of methods for treating a neurological disease in an individual, the density of dendritic spines near amyloid plaques in the brain of the individual is increased.

Neurological diseases include pathologic conditions that affect and impair normal electrical impulses throughout the brain and/or nervous system. General symptoms that may occur during the course of a neurological disease include malfunction of the motor system and sensory network and impairment of normal voluntary and involuntary movement, cognitive function, memory, and abstract thinking. Examples of neurological diseases include Alzheimer's disease, Huntington's disease, Parkinsonism, amyotrophic lateral sclerosis, prion disease, spinocerebellar ataxia, spinal muscular atrophy, autism, autism spectrum disorders, stroke, hypoglycemia, cerebral ischemia, cardiac arrest, spinal cord trauma, head trauma, perinatal hypoxia, cardiac arrest, hypoglycemic neuronal damage, epilepsy, pain, chronic pain, neuropathic pain, fibromyalgia, schizophrenia, depression, bipolar disorder, anxiety, ADHD, dementia, mood disorders, mental disorders, PTSD, sleeplessness, anger management, mania, psychosis, epilepsy, and migraine. In certain preferred embodiments the neurological disease is Alzheimer's disease, Huntington's disease, Parkinsonism, neuropathic pain, amyotrophic lateral sclerosis, prion disease, spinocerebellar ataxia, spinal muscular atrophy, autism, or an autism spectrum disorder. In some embodiments the neurological disease is Alzheimer's disease.

In some embodiments, the neurological disease is characterized by neuroinflammation and microgliosis. Neuroinflammation, or inflammation of the nervous system, involves microglial and astrocyte activation, inflammatory cytokine and reactive oxygen species production, endothelial cell activation, and tissue edema. Neuroinflammation occurs in response to factors including, without limitation, injury, aging, infection, toxins, or autoimmune responses. Neuroinflammation may contribute to neurodegenerative diseases such as Alzheimer's disease by leading to microglial activation, accumulation of amyloid plaques, and synapse loss. Microgliosis involves the abnormal proliferation or hypertrophy of microglia in response to injury or activation signals.

In one aspect of the disclosure, a method of treating an individual exhibiting one or more symptoms of a neurological disease is provided. In some embodiments, the one or more symptoms include, without limitation, memory loss, confusion, disorientation, mood changes, behavior changes, muscle weakness, motor dysfunction, ataxia, speech changes, dementia, rigidity, muscle wasting, tremors, paralysis, repetitive behaviors, communication difficulties, and social skill difficulties. In some embodiments, the one or more symptoms improve after administration of an effective amount of the anti-IL-34 antibody. In some embodiments, the one or more symptoms are measured using the Mini-Mental State Examination. The Mini-Mental State Examination (MMSE) or Folstein test is a brief 30-point questionnaire test that is used to assess cognition. In the time span of about 10 minutes it samples various functions including memory and orientation. The MMSE test includes simple questions and problems in a number of areas: the time and place of the test, repeating lists of words, language use and comprehension, and basic motor skills. Any score of 27 or higher (out of 30) is effectively normal; 20-26 indicates mild dementia; 10-19 moderate dementia, and below 10 severe dementia. The MMSE is a standardized test.

In some aspects of the present disclosure, methods for treating a neurological disease in an individual or methods for treating an individual exhibiting one or more symptoms of a neurological disease are provided. In some embodiments, the neurological disease is Alzheimer's disease (AD). AD is a condition characterized by slowly progressive dementia and gross cerebral cortical atrophy. The incidence of AD averages between four and five percent of the U.S. population. This translates to approximately 1.3 million cases of severe AD and an additional 2.8 million patients with mild to moderate impairment. The presence of β-amyloid neuritic plaques, intraneuronal neurofibrillary tangles, and amyloid angiopathy are hallmarks of AD and are observed at postmortem examination. AD may be heritable in a Familial manifestation, or may be sporadic. AD includes familial, sporadic, as well as intermediates and subgroups thereof based on phenotypic manifestations. Familial AD typically has an early-onset (before age 65) while sporadic AD typically is late-onset (age 65 and later). An individual may be diagnosed as having AD, or at risk of developing AD, by exhibiting phenotypes associated with AD. Phenotypes associated with AD may be cognitive or psychiatric. Examples of cognitive phenotypes include, but are not limited to, amnesia, aphasia, apraxia and agnosia. Examples of psychiatric symptoms include, but are not limited to, personality changes, depression, hallucinations and delusions. Phenotypic manifestations of AD may also be physical, such as by the direct (imaging) or indirect (biochemical) detection of amyloid-β plaques.

Quantitation of amyloid-β (1-40) in the peripheral blood has been demonstrated using high-performance liquid chromatography coupled with tandem mass spectrometry in a linear ion trap (Du et ah, J Biomol Tech. 16(4):356-63 (2005)). Detection of single β-amyloid protein aggregates in the cerebrospinal fluid of Alzheimer's patients by fluorescence correlation spectroscopy also has been described (Pitschke et ah, Nature Medicine 4: 832-834 (1998). U.S. Pat. No. 5,593,846 describes a method for detecting soluble amyloid-β. Indirect detection of amyloid-β peptide and receptor for advanced glycation end products (RAGE) using antibodies also has been described. Lastly, biochemical detection of increased BACE-1 activity in cerebrospinal fluid using chromogenic substrates also has been postulated as a diagnostic or prognostic indicator of AD (Verheijen et al, Clin Chem. April 13 [Epub.] (2006)).

In vivo imaging of β-amyloid can be achieved using radioiodinated flavone derivatives as imaging agents, Ono et al, J Med Chem. 48(23):7253-60 (2005), and with amyloid binding dyes such as putrescein conjugated to a 40-residue radioiodinated A peptide (yielding ¹²⁵I-PUT-A 1-40), which was shown to cross the blood-brain barrier and bind to αβ plaques. Wengenack et al, Nature Biotechnology. 18(8):868-72 (2000). Imaging of β-amyloid was also shown using stilbene SB-13 and the benzothiazole 6-OH-BTA-1 (also known as PIB). Nicholaas et al, Am J Geriatr Psychiatry, 12:584-595 (2004).

In some embodiments, the neurological disease is Parkinson's disease. Parkinson's disease is a chronic, progressive central nervous system disorder which usually appears in the latter decades of life. The disease produces a slowly increasing disability in purposeful movement. It is characterized by four major clinical features of tremor, bradykinesia, rigidity and a disturbance of posture. Often patients have an accompanying dementia. In idiopathic Parkinsonism, there is usually a loss of cells in the substantia nigra, locus ceruleus, and other pigmented neurons of the brain, and a decrease of dopamine content in nerve axon terminals of cells projecting from the substantia nigra. After a number of years the disability, bradykinesia, weakness and rigidity progress to the point of complete invalidism.

In some embodiments, the neurological disease is Huntington's disease. Huntington's Disease (HD), also known as Huntington's Chorea, is a progressive disorder of motor, cognitive and psychiatric disturbances. The mean age of onset for this disease is age 35-44 years, although in about 10% of cases, onset occurs prior to age 21, and the average lifespan post-diagnosis of the disease is 15-18 years. Prevalence is about 3 to 7 for every 100,000 people of western European descent. The disease is caused by an autosomal dominant mutation on either of the two copies of a gene located on the short arm of chromosome 4 at 4p16.3, called huntingtin (htt).

HD is one of several diseases which involve a trinucleotide repeat, leading to the presence of a repeated section in the htt gene. The repeat is that of a sequence of three DNA bases, cytosine-adenine-guanine (CAG), (i.e. . . . CAGCAGCAG . . . ), CAG being the triplet which encodes the amino acid glutamine. Thus, the CAG series results in the production of a chain of glutamines known as a polyglutamine tract (or polyQ tract), and the repeated part of the gene is identified as the polyQ region. This causes striatal and cortical degeneration.

In some embodiments, the neurological disease is neuropathic pain. Neuropathic pain is a chronic condition in which NMDA receptors in neural pain pathways have an abnormally high level of sensitivity so that they spontaneously convey nerve messages that the patient perceives as pain even though no painful stimulus has been inflicted. Neuropathic pain includes any form of pain associated with a neuropathic disease or condition caused by injury or primary irritation of a nerve, including degenerative, toxic, metabolic, ischaemic, and mechanical forms of injury. Neuropathic conditions include all forms of neuritis and polyneuritis. The neuropathic conditions can be hereditary, such as hereditary sensorimotor neuropathy and hereditary sensory and autonomic neuropathy. Neuropathy can also be a result of non-neuropathic conditions such as diabetes (diabetic neuropathy), rheumatic disease, viral infection, multiple sclerosis, some strokes, nutritional deficiencies, metabolic disorders, immune-mediated disorders, and cancer. Myofacial pain is a form of neuropathic pain. One of the distinguishing characteristics of neuropathic pain is that morphine and related pain-killing drugs which are effective in controlling other types of pain are usually ineffective in controlling neuropathic pain (Backonja 1994).

In some embodiments, the neurological disease is amyotrophic lateral sclerosis (ALS). ALS (also called Motor Neuron Disease (MND), Lou Gehrig's disease, or Maladie de Charcot) is a progressive fatal neuromuscular disorder that is characterized by weakness, muscle wasting, and fasciculations (increased reflexes). Cognitive function is retained except where ALS is associated with dementia. The disease primarily affects motor neurons and is characterized by progressive degeneration of the motor neurons in the cerebral cortex, brainstem nuclei and anterior horns of the spinal cord. Individuals afflicted by the disease exhibit weakness of limbs and difficulty in speech and swallowing. The weakness progresses to respiratory impairment, and the disease is usually fatal. Half of all patients die within about 3 years of onset of symptoms. About 5-10% of ALS patients exhibit familial traits. About 20-30% of familial ALS patients exhibit a mutation in their copper/zinc superoxide dismutase (SOD1) gene. However, in greater than 90% of ALS patients, the disease is sporadic and the patients do not exhibit familial traits. Current treatments for ALS are only palliative.

In some embodiments, the neurological disease is a prion disease. Prion diseases are a group of rapidly progressive, fatal, and untreatable neurodegenerative syndromes. Human prion diseases include classical Creutzfeldt-Jakob disease (CJD), which has sporadic, iatrogenic, and familial forms. More recently, a variant CJD (vCJD) has been recognized in the United Kingdom, France, the Republic of Ireland, Hong Kong, Italy and the United States, likely derived from the consumption of cattle tissues contaminated with the agent of bovine spongiform encephalopathy (BSE). The prion diseases are neurodegenerative syndromes characterized by spongiform change (e.g., microcavitation of the brain, usually predominant in gray matter), neuronal cell loss, astrocytic proliferation disproportionate to neuronal loss, and accumulation of an abnormal amyloidogenic protein, sometimes in discrete plaques in the brain. Prions, the infectious agents that transmit these diseases differ markedly from viruses and viroids in that no chemical or physical evidence for a nucleic acid component has been reproducibly detected in infectious materials.

In some embodiments, the neurological disease is spinocerebellar ataxia (SCA). SCAs are a complex group of heterogeneous autosomal dominant neurodegenerative disorders characterized by cerebellar dysfunction alone or in combination with other neurological abnormalities. In spinocerebellar ataxia, the expansions of CAG trinucleotide repeats encoding a polyglutamine (polyQ) stretch have been shown to cause dominantly inherited SCA1, SCA2, SCA3, SCA6, SCAT, SCA17 and dentatorubropallidoluy-sianatrophy (DRPLA). These polyQ-mediated genetic disorders in SCAs have shown selective progressive degeneration of the cerebellum, brainstem, and spinal cord tract, with the prominent pathological hallmark of intranuclear and cytoplasmic accumulation of aggregated polyQ proteins inside degenerated neurons, thereby causing the dysfunction and degeneration of specific neurons. The clinical symptoms include ataxia, dysarthria, ophthalmoparesis, and variable degrees of motor weakness. The symptoms usually begin during the third or fourth decade of life, however, juvenile onset has been identified. Typically, the disease worsens gradually, often resulting in complete disability and death 10-20 years after the onset of symptoms. Individuals with juvenile onset spinocerebellar ataxias, however, typically have more rapid progression of the phenotype than the late onset cases.

In some embodiments, the neurological disease is spinal muscular atrophy (SMA). SMA is an autosomal recessive neurological disorder that results from loss of function of the anterior horn cells in the spinal cord, manifesting as progressive motor weakness, muscle wasting, and paralysis. SMA is caused by insufficient levels of the survival motor neuron (SMN) protein. The SMN locus on chromosome 5q13 contains two inverted copies of SMN called SMN1 and SMN2. Most cases of SMA harbor homozygous deletions of the SMN1 gene and retain at least one copy of SMN2. SMA manifests across a continuous spectrum of severity and age of disease onset that has been divided into four groups: type I (severe infantile acute SMA, or Werdnig-Hoffmann disease); type 11, (infantile chronic SMA); type III (juvenile SMA, or Wohlfart-Kugelberg-Welander disease); and type IV (adult-onset SMA).

In some embodiments, the neurological disease is autism or autism spectrum disorders. Autism spectrum disorders (ASDs) are pervasive neurodevelopmental disorders diagnosed in early childhood when acquired skills are lost or the acquisition of new skills becomes delayed. ASDs onset in early childhood and are associated with varying degrees of dysfunctional communication and social skills, in addition to repetitive and stereotypic behaviors. In many cases (25%-50%), a period of seemingly normal development drastically shifts directions as acquired skills are lost or the acquisition of new skills becomes delayed. In recent years, the number of people with an ASD has increased considerably to approximately 1 in 150 children. Although the neurobiological basis for autism remains poorly understood, several lines of research now support the view that genetic, environmental, neurological, and immunological factors contribute to its development.

In a further aspect, the present disclosure provides a method for reducing the density of microglia in the brain. In some embodiments, the microglia density is reduced by at least 30%, at least 40%, at least 50%, at least 60%, at least 70%, or at least 80%. Microglia are resident brain and spinal cord macrophages involved in central nervous system development and homeostasis. They compose 10-15% of brain cells and reside throughout the central nervous system, including the brain, spinal cord, and retina. Microglia use phagocytic and cytotoxic mechanisms to destroy dead cells and infectious agents present in the central nervous system. Microglia also enhance the immune response by functioning as antigen-presenting cells and secreting cytokines and signaling molecules.

Antibodies of the present disclosure can be used either alone or in combination with other agents in a method of treatment. For instance, an antibody of the present disclosure may be co-administered with at least one additional therapeutic agent. In some embodiments, an additional therapeutic agent is a CSF-1R inhibitor. In some embodiments, the CSF-1R inhibitor is a small molecule inhibitor. In some embodiments, the small molecule inhibitor is GW2580. GW2580 is commercially available from FISHER SCIENTIFIC, INC. In some embodiments, the CSF-1R inhibitor is an anti-CSF-1R antibody. In some embodiments, an additional therapeutic agent is an anti-CSF1-antibody.

Such combination therapies noted above encompass combined administration (where two or more therapeutic agents are included in the same or separate formulations), and separate administration, in which case, administration of the antibody of the present disclosure can occur prior to, simultaneously, and/or following, administration of the additional therapeutic agent and/or adjuvant.

An antibody of the present disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the present disclosure are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the present disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the antibody). An initial higher loading dose, followed by one or more lower doses may be administered. An exemplary dosing regimen comprises administering. However, other dosage regimens may be useful. The progress of this therapy is easily monitored by conventional techniques and assays.

F. Articles of Manufacture or Kit

In another aspect of the present disclosure, an article of manufacture or kit including a pharmaceutical composition containing an anti-IL-34 antibody and a pharmaceutically acceptable carrier is provided. In some embodiments the pharmaceutical composition further contains an inhibitor of CSF-1R. In some embodiments the inhibitor of CSF-1R is a small molecule inhibitor. In some embodiments the small molecule inhibitor is GW2580. In another embodiment the inhibitor of CSF-1R is an anti-CSF-1R antibody.

In some embodiments, the kit contains instructions for administering an effective amount of the pharmaceutical composition to an individual for treating a neurological disease. In some embodiments, the neurological disease is selected from, without limitation, Alzheimer's disease, Parkinson's disease, Huntington's disease, amyotrophic lateral sclerosis, neuropathic pain, prion disease, spinocerebellar ataxia, spinal muscular atrophy, autism, and autism spectrum disorders. In some embodiments, the neurological disease is Alzheimer's disease.

The article of manufacture or kit typically includes a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease and may have a sterile access port (for example the container may be an intravenous solution bag or a vial having a stopper pierceable by a hypodermic injection needle). At least one active agent in the composition is an antibody of the present disclosure. The label or package insert indicates that the composition is used for treating the disease of choice. Moreover, the article of manufacture or kit may comprise (a) a first container with a composition contained therein, wherein the composition comprises an antibody of the present disclosure; and (b) a second container with a composition contained therein, wherein the composition comprises an additional therapeutic agent. The article of manufacture in this embodiment of the present disclosure may further comprise a package insert indicating that the compositions can be used to treat a particular disease. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### Example 1: Anti-IL-34 Antibody Decreased Microglia Density

The CSF1R signaling pathway has been shown to be required for microglia proliferation and survival (Gomez-Nicola et al, 2013, Elmore et al, 2014). The effect of inhibiting the CSF1R signaling pathway on microglia density and shape was evaluated. The CSF1R pathway was inhibited by either targeting the receptor with a small molecule inhibitor and/or with a neutralizing antibody to one of its ligands, IL-34.

### Methods

Il-34 Depletion

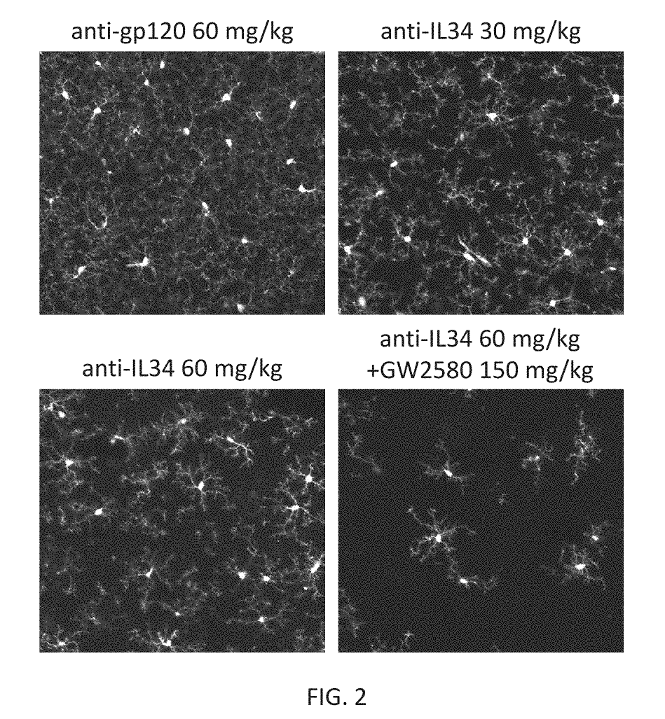

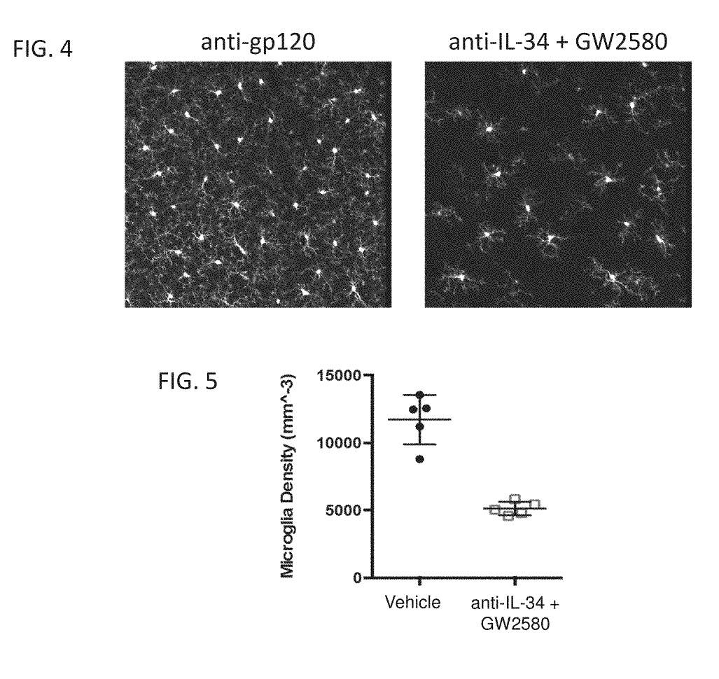

2 month old male CX3CR1-GFP mice, which express GFP in microglia, were dosed intraperitoneally (IP) with anti-IL-34 antibody (30 or 60 mg/kg) or anti-gp120 (control mIgG2a antibody, 60 mg/kg) twice per week for 3 weeks. An additional group of mice received a combination of anti-IL-34 antibody (60 mg/kg), dosed as described above, and CSF1R small molecule inhibitor GW2580 (150 mg/kg), dosed per oral (PO) once (FIG. 4) or twice (FIG. 2) per day for 3 weeks. 5 mice were treated per group.

Microglia Imaging

Mice were anesthetized, perfused with saline, and brains were collected and fixed in 4% paraformaldehyde+10% sucrose at 4 degrees Celsius overnight. After fixation, brains were embedded in agarose and immersed in PBS, then imaged en bloc using a 2-photon microscope (Prairie Technologies Ultima IV microscope powered by a Spectra Physics MaiTai DeepSee laser) with a 20× immersion objective (Olympus). Imaging was performed at a field-of-view of 1024 by 1024 pixels and z-axial step size of 1.5 μm through a depth of 100 μm in the somatosensory cortex using 910 nm laser wavelength. Microglia density and morphometry were quantified in MATLAB (Mathworks) using custom image analysis routines.

### Results

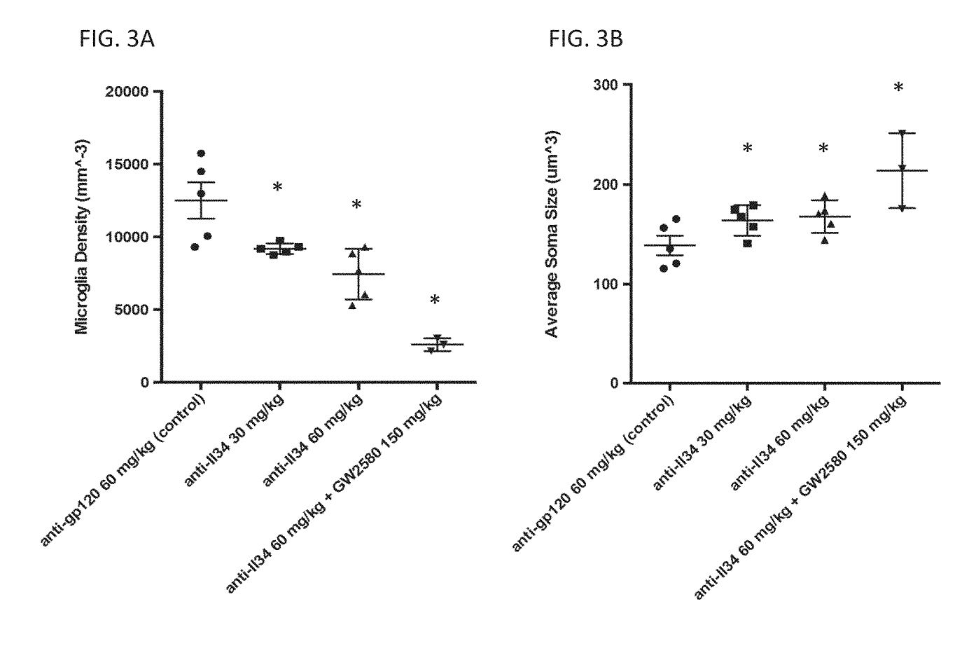

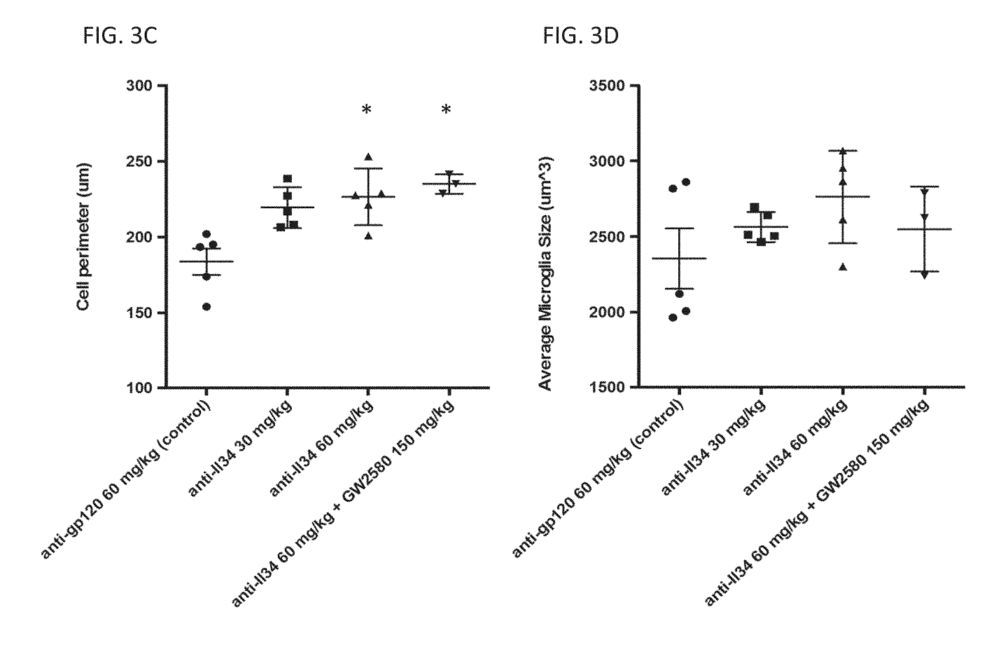

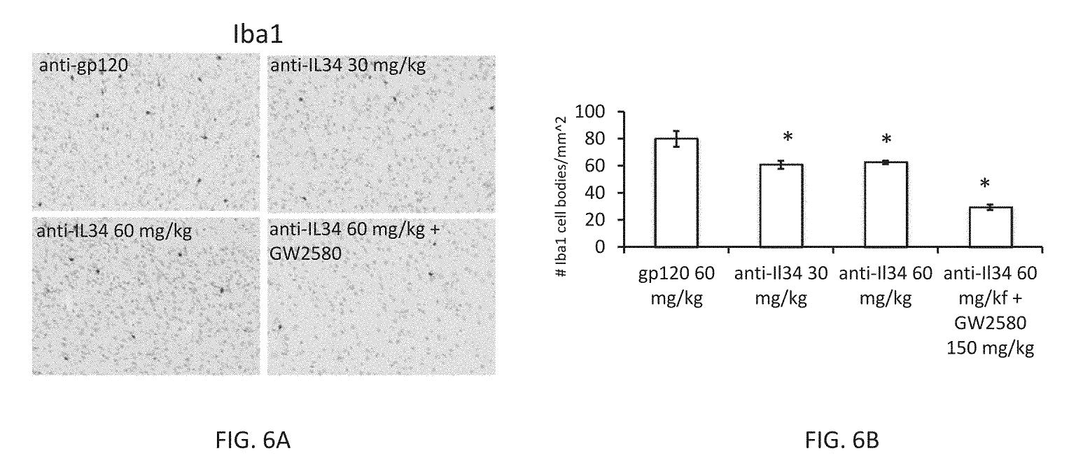

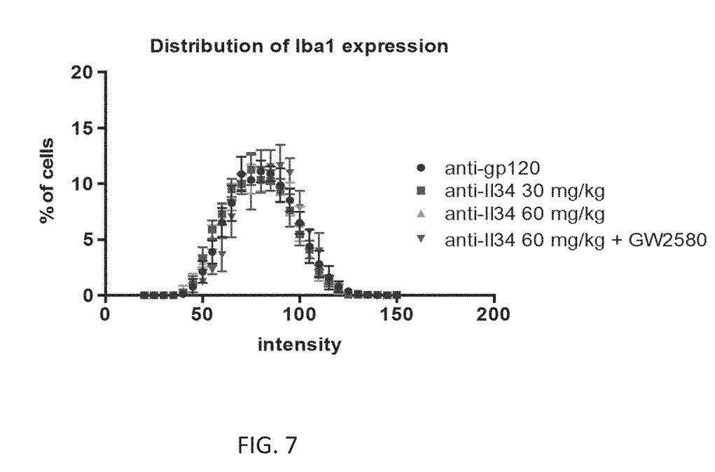

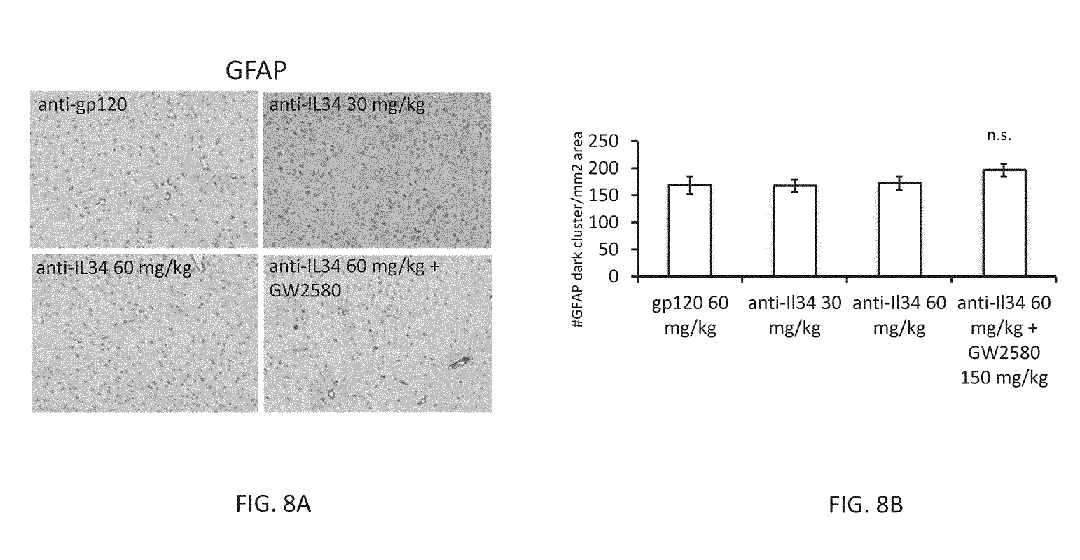

Anti-IL-34 antibody treatment, both alone and in combination with GW2580, reduced microglia density compared to anti-gp120 control antibody (FIG. 2 and FIG. 4). A dose dependent effect was observed; the 30 and 60 mg/kg doses reduced microglia density by about 27% and 40%, respectively. Combined anti-IL-34 and GW2580 treatment reduced microglia density by about 60% when dosed once per day and about 80% when dosed twice per day (FIG. 3A and FIG. 5). Anti-IL-34 antibody treatment, both alone and in combination with GW2580, altered microglia shape, as indicated by an increased average soma size (FIG. 3B), increased cell perimeter (FIG. 3C), and increased average microglia size (FIG. 3D). No evidence of microglia activation or astrogliosis in response to microglia depletion was observed (FIGS. 6A and 6B, FIG. 7, FIGS. 8A and 8B). Microglia depletion was also observed in the spinal cord.

### Example 2: Anti-IL-34 Antibody Decreased Microglia Density, Whereas Anti-CSF1 Antibody had No Effect on Microglia Density

The effect of an anti-IL-34 antibody or an anti-CSF1 antibody on microglia density and shape was evaluated.

### Methods

IL-34 and CSF Depletion

2 month old male CX3CR1-GFP mice, which express GFP in microglia, were dosed intraperitoneally (IP) with anti-IL-34 antibody (10 or 100 mg/kg), anti-CSF1 antibody (10 or 100 mg/kg), anti-IL-34 antibody (10 mg/kg) plus anti-CSF1 antibody (10 mg/kg), or anti-gp120 (control mIgG2a antibody, 100 mg/kg) twice per week for 3 weeks. An additional group of mice received a combination of anti-IL-34 antibody (60 mg/kg), dosed as described above, and CSF1R small molecule inhibitor GW2580 (150 mg/kg), dosed per oral (PO) once daily for 21 days. 5 mice were treated per group. Microglia imaging was performed in the somatosensory cortex as described in Example 1, from the cortical surface down through 100 microns with 1.5 micron steps.

### Results

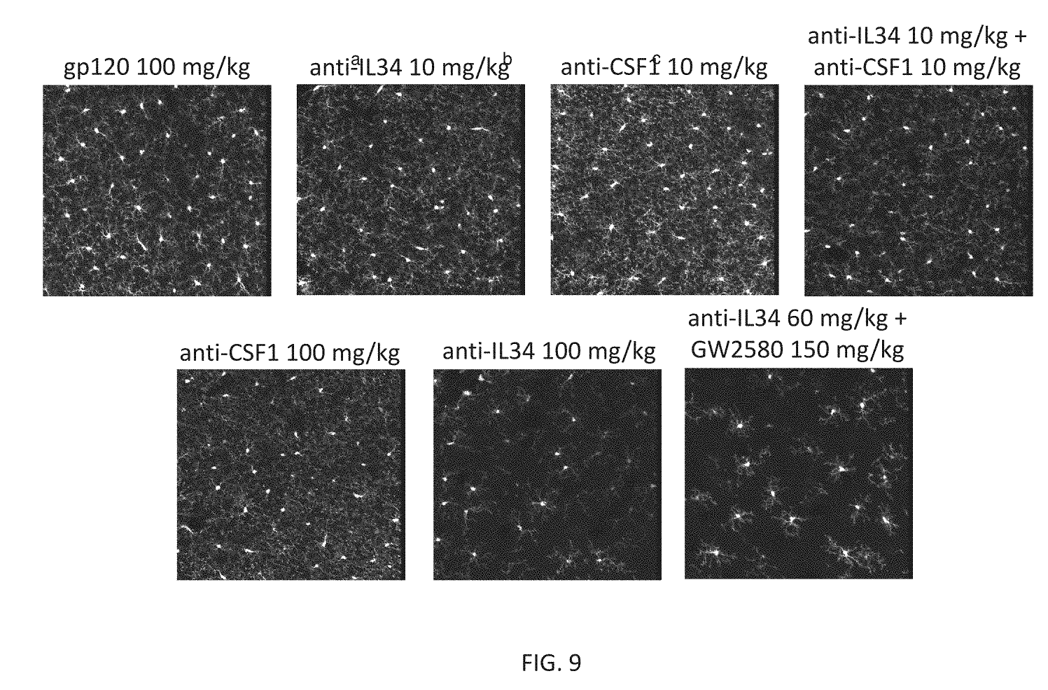

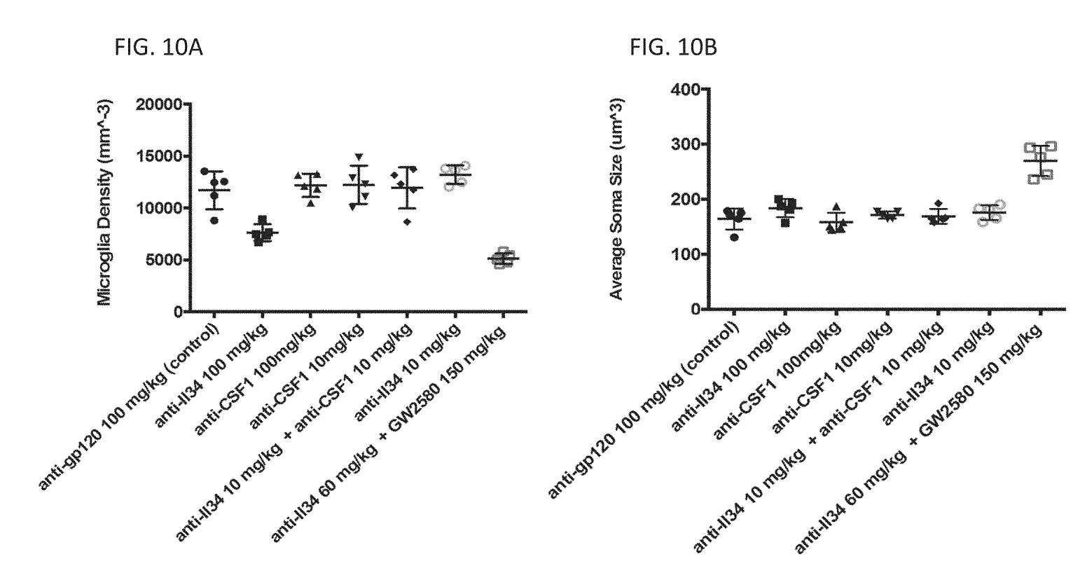

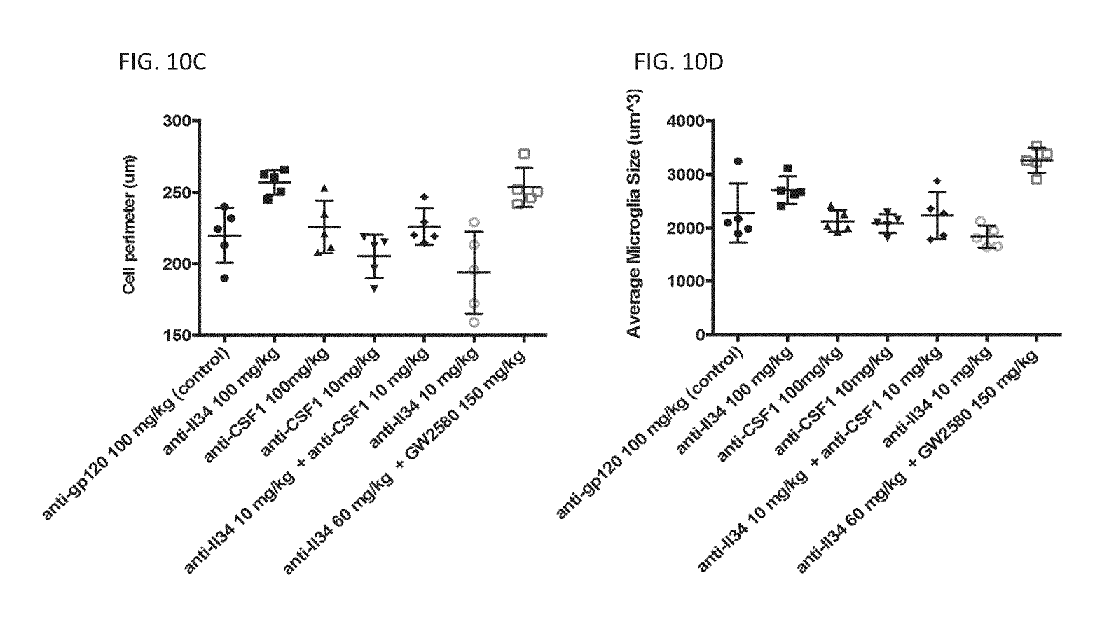

Anti-IL-34 antibody treatment (100 mg/kg dose) and anti-IL-34 plus GW2580 treatment reduced microglia density compared to anti-gp120 control antibody (FIG. 9 and FIG. 10A). Anti-CSF1 antibody treatment, either alone or in combination with anti-IL-34 antibody, had no effect on microglia density (FIG. 9 and FIG. 10A). Anti-IL-34 antibody treatment alone (100 mg/kg dose) and anti-IL-34 plus GW2580 treatment altered microglia shape, as indicated by an increased cell perimeter (FIG. 10C) and increased average microglia size (FIG. 10D). Anti-IL-34 plus GW2580 treatment also increased average soma size (FIG. 10B). A trend towards increased soma size was observed in the anti-IL-34 100 mg/kg group, but it did not reach statistical significance (FIG. 10B). Anti-CSF1 antibody treatment, either alone or in combination with anti-IL-34 antibody, had no effect on microglia shape (FIGS. 10B-10D). Without wishing to be bound by theory, the results described herein with regard to the anti-CSF1 antibody treatment may have been affected by an incorrectly folded or an incorrectly purified anti-CSF1 antibody protein. Microglia cell spread, eccentricity, roundness, and average microglia intensity measurements indicated a healthy cellular phenotype.

### Example 3: Anti-IL-34 Antibody and Anti-CSF1 Antibody Decreased Microglia Density in Different Regions of the Brain

The effect of an anti-IL-34 antibody or an anti-CSF1 antibody on microglia density in different regions of the brain was evaluated.

### Methods

IL-34 and CSF-1 Depletion

2 month old male CX3CR1-GFP mice, which express GFP in microglia, were dosed intraperitoneally (IP) with anti-IL-34 antibody (60 mg/kg), anti-CSF1 antibody (100 mg/kg), anti-IL-34 antibody (60 mg/kg) plus anti-CSF1 antibody (100 mg/kg), or anti-gp120 (control mIgG2a antibody, 60 mg/kg) twice per week for 3 weeks. An additional group of mice were fed for 21 days with control mouse chow or chow containing Compound X, a receptor tyrosine kinase inhibitor with specificity for CSF-1R and for c-kit, milled into the chow (290 mg/kg chow). 5 mice were treated per group.

### Results

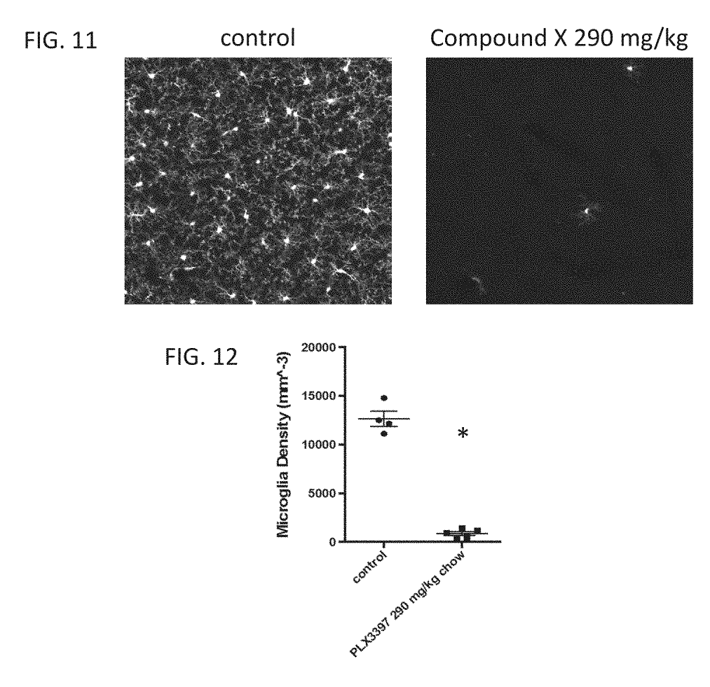

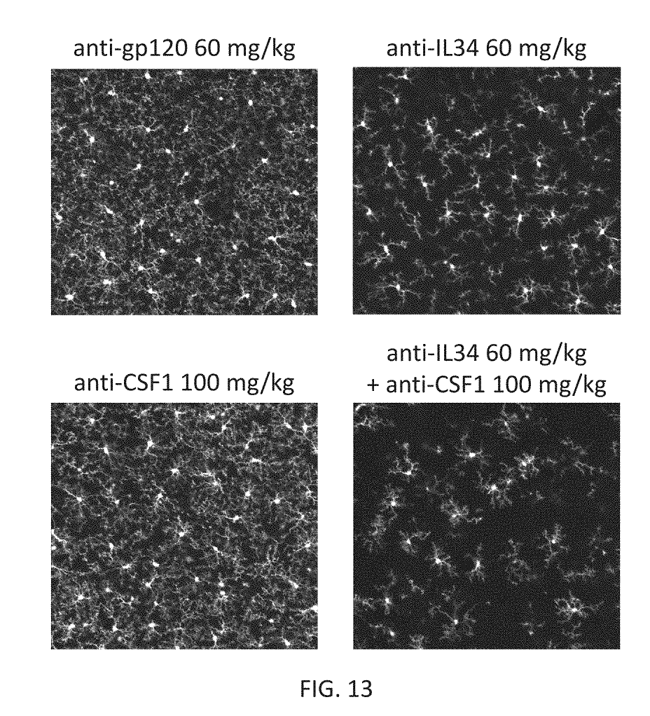

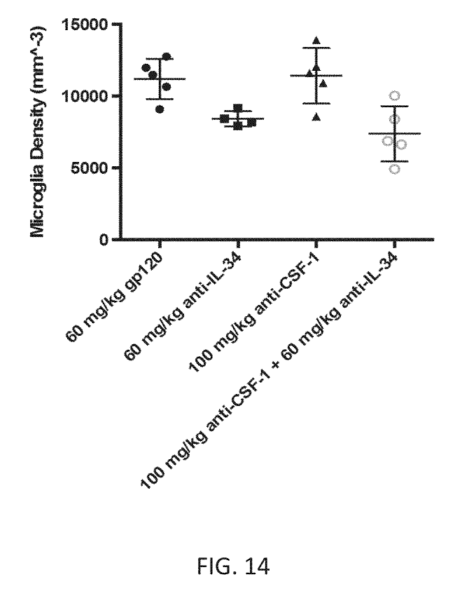

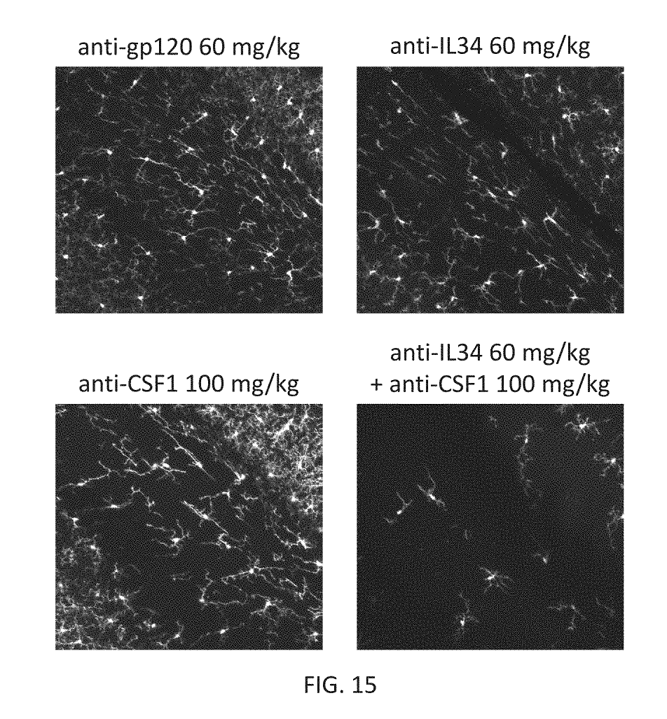

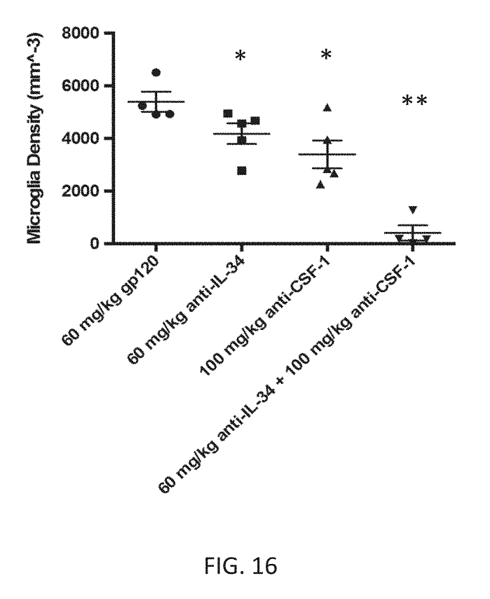

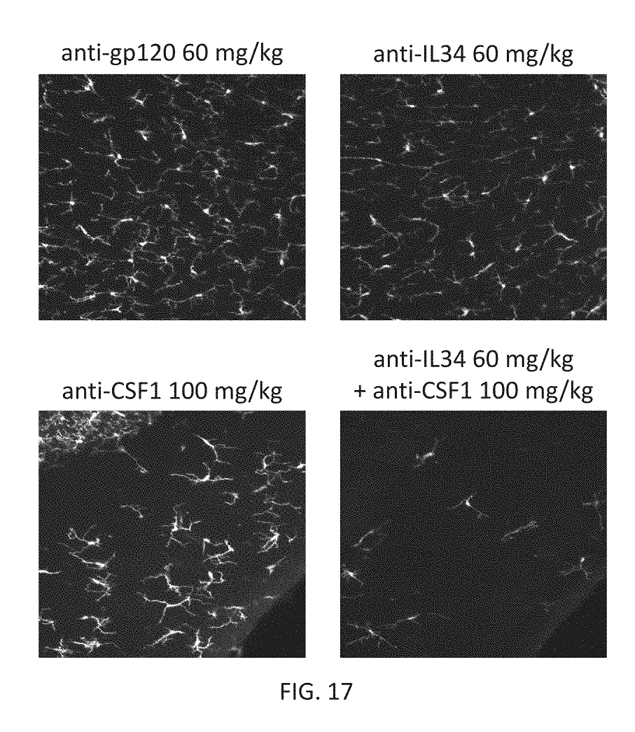

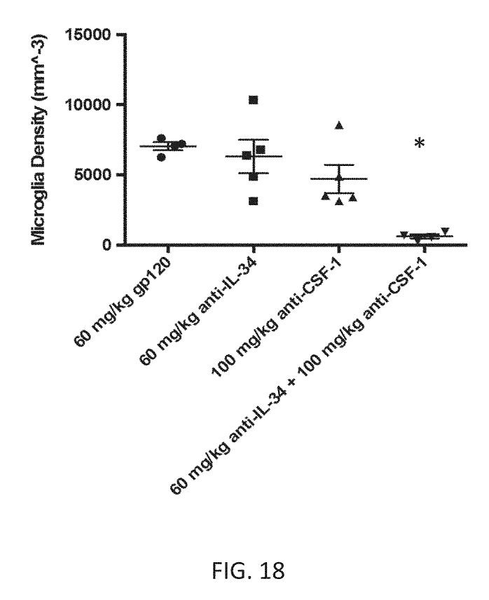

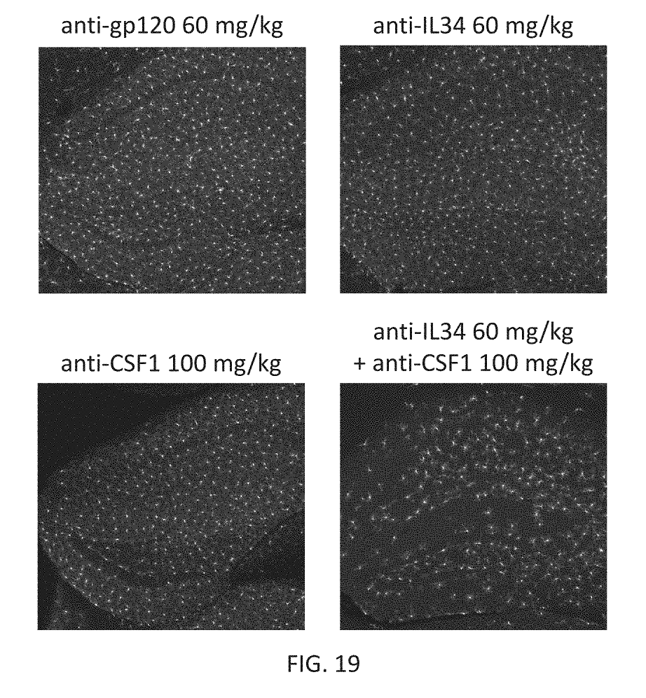

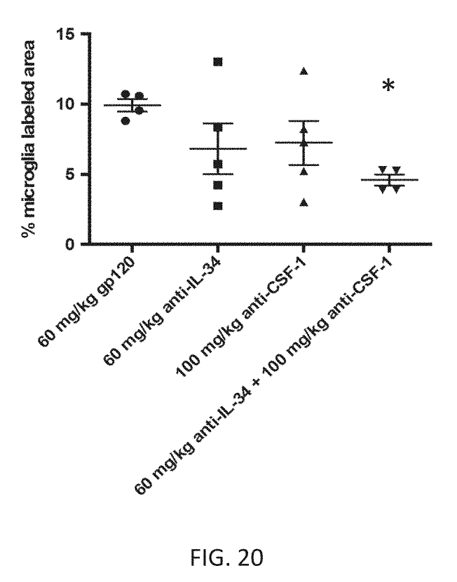

CX3CR1-GFP mice fed for 21 days with mouse chow containing Compound X (290 mg/kg chow) showed a marked reduction of microglia density in cortical gray matter, reducing microglia density >90% compared to mice fed on control chow (FIG. 11 and FIG. 12). To test the effects of antibodies that deplete the two known ligands of CSF1R in cortical gray matter, CX3CR1-GFP mice were treated with anti-IL-34 antibody, anti-CSF1 antibody, anti-IL-34 antibody plus anti-CSF1 antibody, or control antibody. Anti-IL-34 antibody treatment (60 mg/kg) alone, or in combination with anti-CSF1 antibody (100 mg/kg), reduced microglia density in cortical gray matter when compared to anti-gp120 antibody treatment (60 mg/kg), while anti-CSF1 antibody treatment (100 mg/kg) alone had no effect on microglia density in this tissue (FIG. 13 and FIG. 14). In contrast to the results observed in cortical gray matter, anti-CSF1 antibody treatment reduced microglia density in white matter tracts of the corpus callosum, resulting in a greater reduction in microglia density than that observed with anti-IL-34 antibody treatment alone (FIG. 15 and FIG. 16). Treatment of mice with both anti-IL-34 and anti-CSF1 antibodies resulted in an approximately ten-fold reduction in microglia density in white matter of the corpus callosum when compared to the anti-gp120 control antibody treatment. In line with these results, a larger reduction in microglia density was observed in white matter of the hippocampal fimbria from mice treated with anti-CSF1 antibody compared to anti-IL-34 antibody, and an additive effect of treatment with both antibodies was observed (FIG. 17 and FIG. 18). Treatment with anti-IL-34 antibody and anti-CSF1 antibody, alone or in combination, reduced the area taken up by microglia in the hippocampus compared to anti-gp120 antibody treatment (FIG. 19 and FIG. 20).

Taken together, these data suggested that peripheral administration of antibodies that depleted the two known ligands of CSF1R, CSF1 and IL-34, resulted in region-specific depletion of microglia. Treatment with anti-IL-34 antibody reduced microglia in cortical gray matter by approximately 40%, but minimal depletion was observed in white matter tracts. Conversely, treatment with anti-CSF1 antibody had minimal effect on cortical gray matter microglia density, but reduced microglia density in white matter tracts up to 45%. Without wishing to be bound by theory, this data suggests that pharmacological targeting of CSF1R ligands would enable brain region-specific depletion of microglia.

### Example 4: Association of Microglia, Plaques, and Dendritic Spine Loss in Alzheimer's Disease Pathology in a Mouse Model

Microglia constitute 10% of the total cells in the brain, and play numerous roles in both tissue homeostasis and response to damage. Evidence from patients has long implied a role for microglia in Alzheimer's disease (AD).

### Methods

Microglia, Plaque, and Neuron/Synapse Imaging

PS2APP^{+/+} CX3CR1-GFP mice, a model of AD in which GFP is expressed in microglia, and PS2APP^{+/+} GFP-M mice, a model of AD in which GFP is expressed in neurons/synapses, were injected with methoxy-X04 to label amyloid plaques 1 day before takedown. The mice were anesthetized, perfused with saline, followed by 4% PFA. A mixture of gelatin and 1% BSA-AlexaFluor680 was then perfused to label blood vessels. Carcasses are placed on ice to solidify vascular casts, and brains were collected and fixed in 4% paraformaldehyde+10% sucrose at 4 degrees Celsius overnight. After fixation, brains were embedded in agarose and immersed in PBS, then imaged en bloc using a 2-photon microscope (Prairie Technologies Ultima IV microscope powered by a Spectra Physics MaiTai DeepSee laser) with a 20× immersion objective (Olympus). Imaging was performed at a field-of-view of 1024 by 1024 pixels and z-axial step size of 1.5 μm through a depth of 100 μm using 840 nm and 1020 nm laser wavelength. Microglia density and morphometry were quantified in MATLAB (Mathworks) using custom image analysis routines.

EdU Labeling of Microglia

Mice were injected with 5-ethynyl-2′-deoxyuridine (EdU) to label dividing cells. Mice were injected with EdU once per day for three days, IP (50 mg/kg). Three weeks after the final injection the mice were anesthetized and perfused with saline, followed by 4% PFA, and brains were collected and fixed in 4% paraformaldehyde+10% sucrose at 4 degrees Celsius overnight. Immunohistochemitry for Iba1 (Wako Chemical, item #019-19741) and a Click-it reaction to detect EdU (Thermo Fischer Scientific, item #C10338) were performed. Images were captured on a Zeiss LSM710 confocal with a 63× objective.

### Results

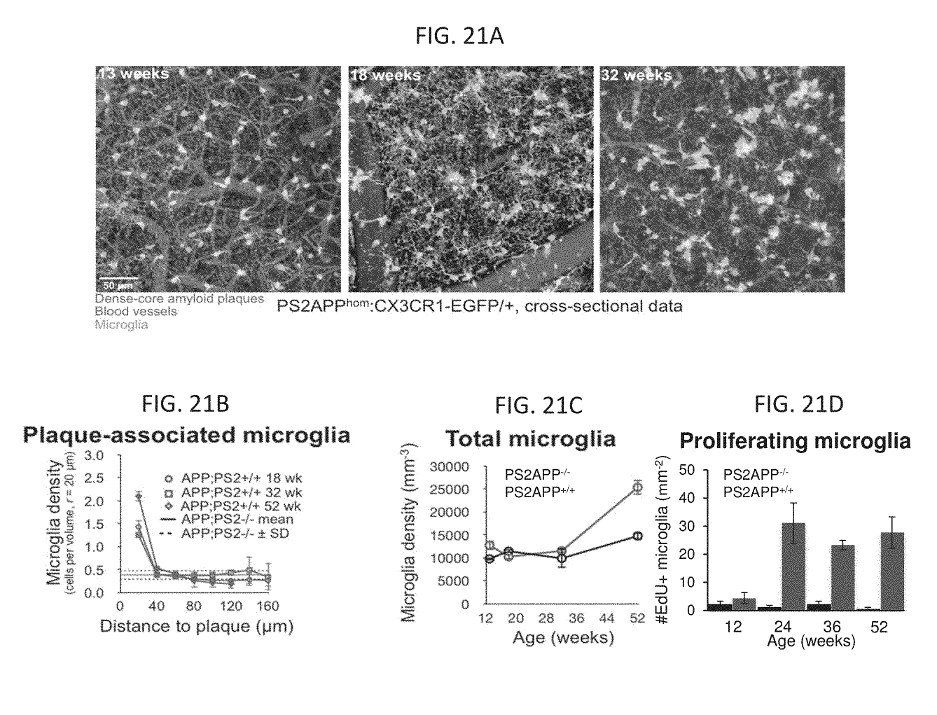

Microglia, blood vessels, and dense-core amyloid plaques were imaged in PS2APP^{+/+} CX3CR1-GFP mice between 13 and 32 weeks of age (FIG. 21A). Increased dense-core amyloid plaque formation was observed in the older mice, with a concomitant increase in microglia numbers. To further characterize the association of microglia and amyloid plaques in older mice, microglia density was measured as a function of distance from the amyloid plaques in PS2APP^{+/+} CX3CR1-GFP mice between 18 and 52 weeks of age (FIG. 21B). Interestingly, a substantial increase in microglia density was observed within 40 μm of the amyloid plaques, suggesting significant accumulation of microglia around these plaques. Beginning around 32 weeks of age, a significant increase in total microglia numbers were observed in the brains of PS2APP^{+/+} CX3CR1-GFP relative to their wild-type counterparts (FIG. 21C), with a sharp rise in microglia proliferation observed beginning at about 24 weeks of age in PS2APP^{+/+} mice (FIG. 21D).

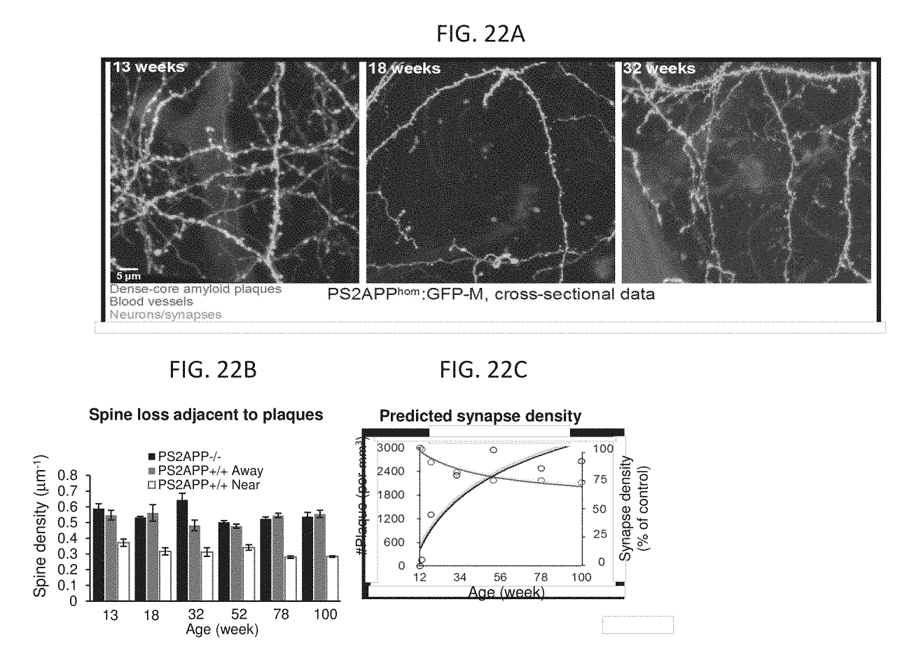

To test the effects of plaques on dendritic spine loss and synapse density, neurons/synapses, blood vessels, and dense-core amyloid plaques were imaged in PS2APP^{+/+} GFP-M mice (FIG. 22A). As these mice aged, dendritic spine density decreased near the dense-core amyloid plaques, but the dendritic spine density far away from the plaques was similar to PS2APP^{-/-} mice (FIG. 22B). A strong correlation was observed between plaque number/density and synapse density as the PS2APP^{+/+} GFP-M mice aged (FIG. 22C and FIG. 22D). An approximate 33% decrease in synapse density was observed for synapses within 40 μm of amyloid plaques. Taken together, this data suggests that dendritic loss is focal to dense-core amyloid plaques, and the increased microglia proliferation/accumulation observed near dense-core amyloid plaques is coincident with dendritic spine loss.

### Example 5: Effect of Anti-IL-34 Antibody on Alzheimer's Disease Pathology in a Mouse Model

Microglia constitute 10% of the total cells in the brain, and play numerous roles in both tissue homeostasis and response to damage. Evidence from patients has long implied a role for microglia in Alzheimer's disease (AD). The effect of depleting microglia in a mouse model of AD was evaluated.

### Methods

Il-34 Depletion

PS2APP^{+/+} CX3CR1-GFP mice, a model of AD in which GFP is expressed in microglia, were treated with GW2580 and anti-IL-34 to deplete microglia. 5 treatment groups were utilized, with 10 PS2APP mice per group. Table 2 shows the antibody dosages for each treatment group.

TABLE 2
PS2APP^{+/+} CX3CR1-GFP mice treatment groups

| Group | Treatment |
|---|---|
| 1 | vehicle + 60 mg/kg anti-gp120 |
| 2 | 150 mg/kg GW2580 + 60 mg/kg anti-IL-34 |
| 3 | vehicle + 60 mg/kg anti-gp120 |
| 4 | 150 mg/kg GW2580 + 60 mg/kg anti-IL-34 |
| 5 | 150 mg/kg GW2580 + 60 mg/kg anti-IL-34 |

Mice were treated with CSF1R small molecule inhibitor GW2580 (suspended in MCT), PO, once per day at 150 mg/kg (<0.25 mL), and anti-IL-34 antibody, IP, twice per week at 60 mg/kg. Control mice were treated with vehicle PO and anti-gp120 following the same dosing schedule.

Groups 1 and 2 were dosed for 4 weeks and euthanized 3-8 hours after the final dose of GW2580 or vehicle was administered for tissue collection. Groups 3 and 4 were dosed for 8 weeks and euthanized 3-8 hours after the final dose of GW2580 or vehicle was administered for tissue collection. Group 5 was dosed for 4 weeks, allowed to recover for 4 weeks after the final dose was administered, and euthanized.

Open Field Assessment of Activity

The effects of treatment on general activity were assessed by testing mice in the open field. Assessment of spontaneous locomotor activity in an open field reveals changes in neural transmission, motor function and/or learning and memory. Mice were placed in a novel open chamber (40 cm×40 cm) made of gray plastic and allowed to explore it freely for 15 minutes. Activity was recorded by video tracking from a camera mounted overhead.

This test is performed prior to dosing and 1-2 h after dosing on the final week of treatment in order to assess the time course of habituation to the environment. A maximum of 1 trial/day was conducted for each mouse. Since the animal is free to move about the chamber for the duration of the test, no adverse effects or significant discomfort to the animal was expected.

AD Disease Pathology

The effect of microglia depletion on AD pathology in the PS2APP mouse model, including dendritic spine density, dendritic arborization, and amyloid plaque density/size, was evaluated.

### Results

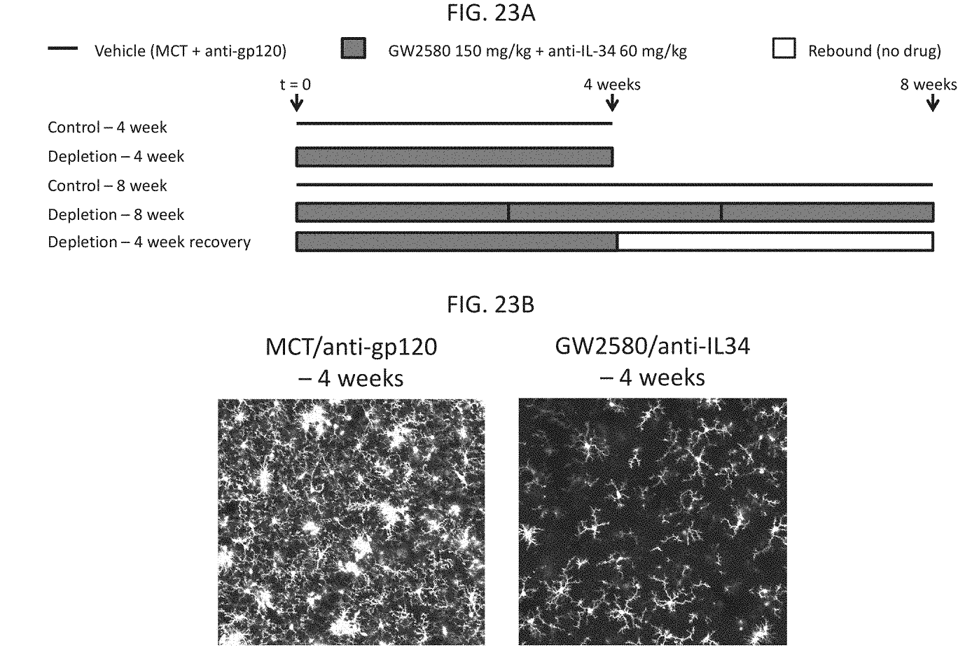

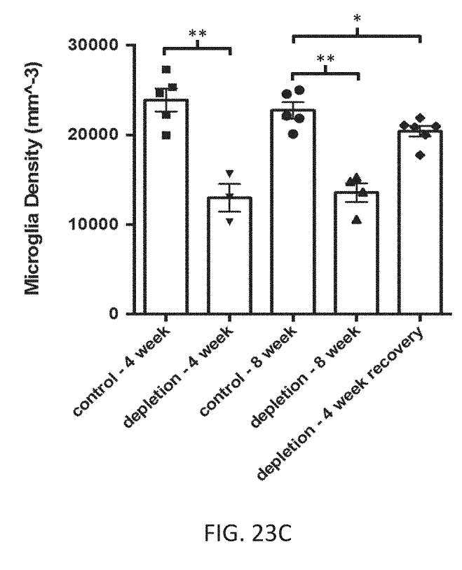

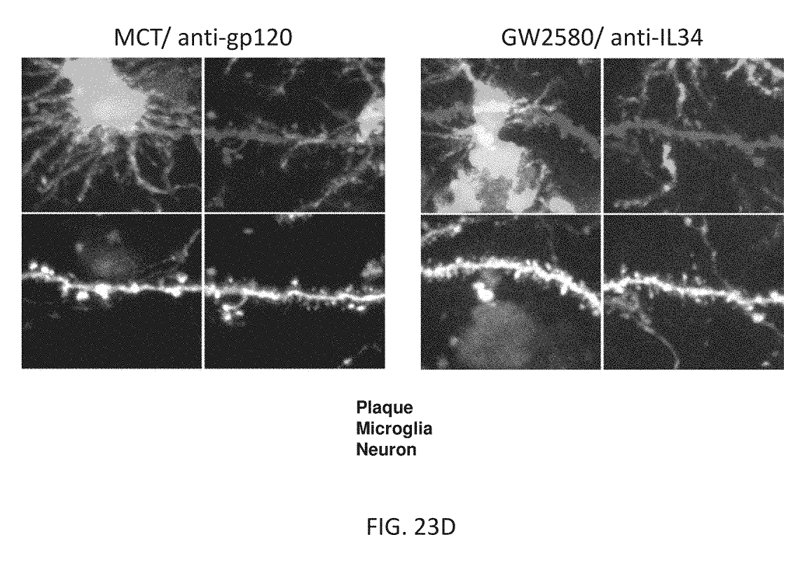

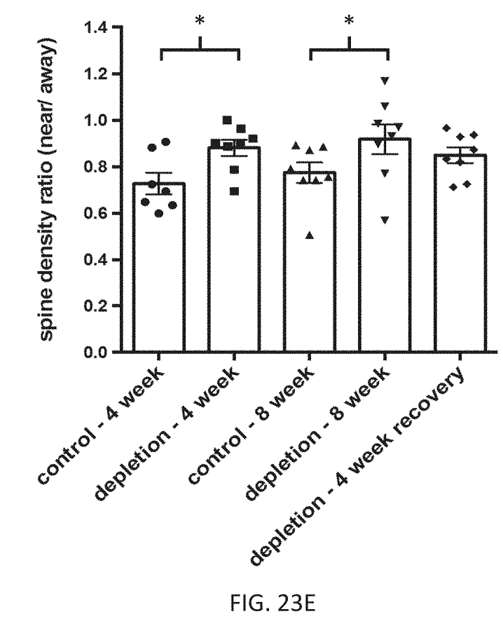

5 groups (as described in table 2 above) of PS2APP^{+/+} CX3CR1-GFP mice were treated with a combination of CSF1R small molecule inhibitor GW2580 plus anti-IL-34 antibody (depletion, GW2580/anti-IL-34), or vehicle plus anti-gp120 antibody (control, MCT/anti-gp120) following the dosing schedule described in FIG. 23A. Mice treated with the CSF1R small molecule inhibitor GW2580 plus anti-IL-34 antibody for four weeks showed reduced microglia density when compared to mice treated with the vehicle plus anti-gp120 antibody control (FIG. 23B). Mice treated for four or eight weeks with the CSF1R small molecule inhibitor GW2580 plus anti-IL-34 antibody had an approximate two-fold reduction in microglia density relative to the control four and eight week treated mice (FIG. 23C). Mice treated for four weeks with the CSF1R small molecule inhibitor GW2580 plus anti-IL-34 antibody, and then allowed to rebound for four weeks with no drug treatment, revealed that while microglia density in these mice did not recover to the levels observed in the control group, microglia density increased significantly relative to both the four and eight week depletion groups (FIG. 23C).

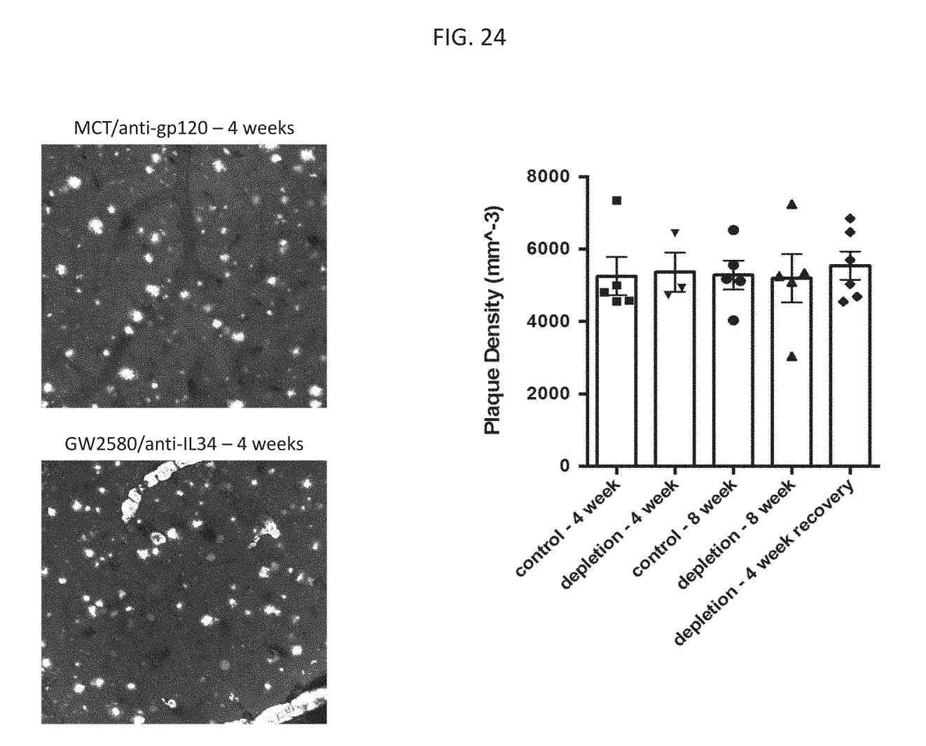

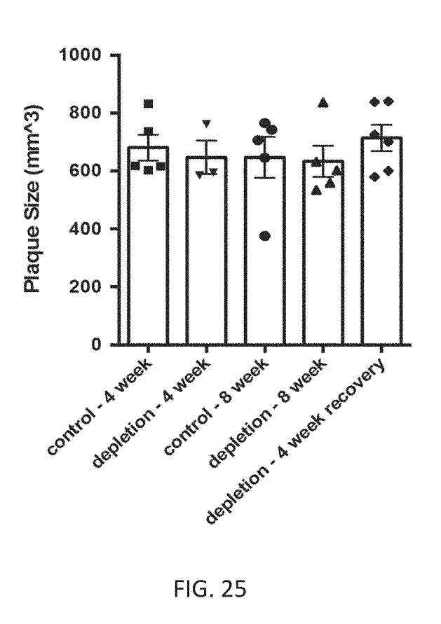

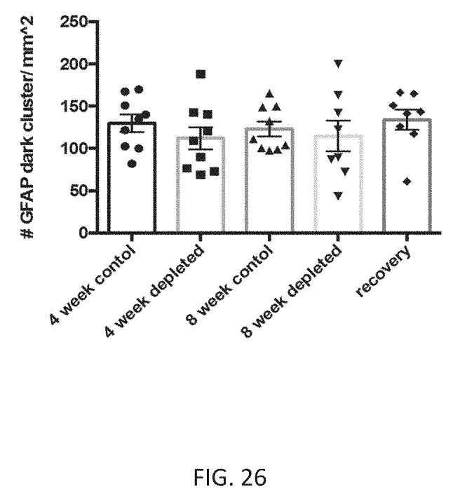

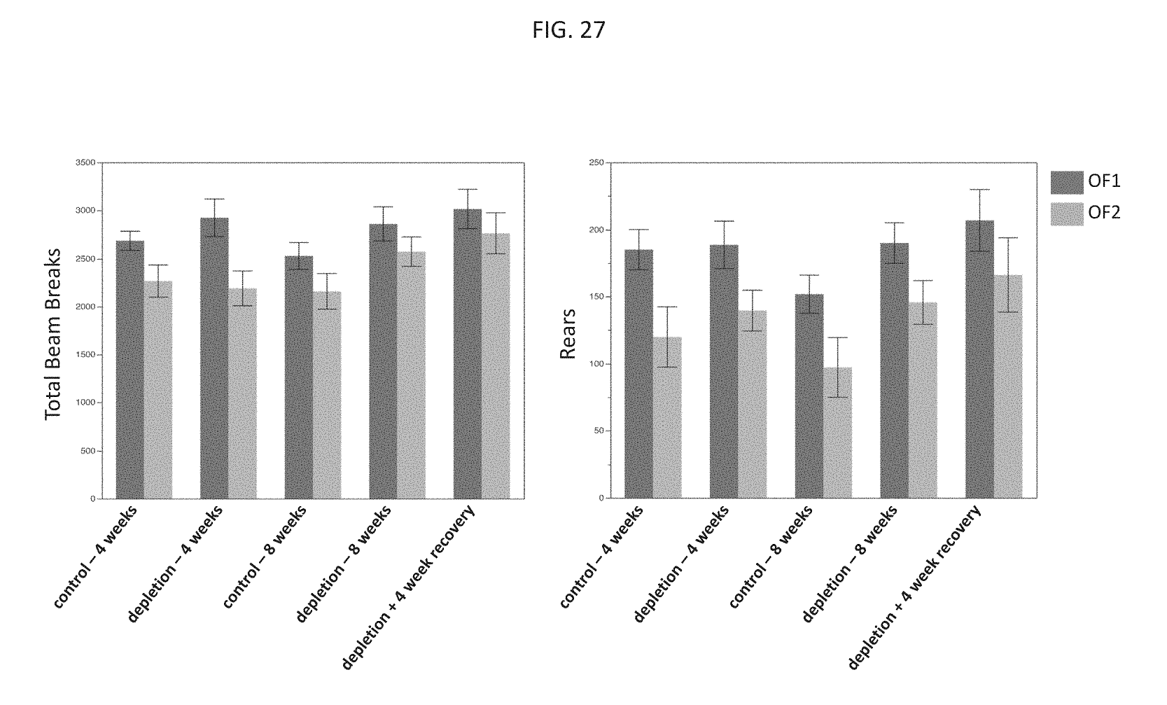

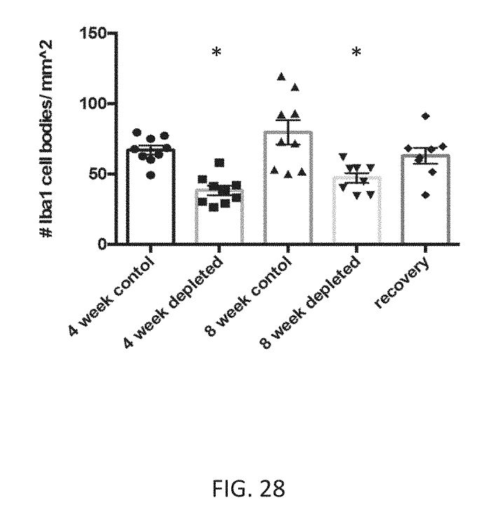

To test the effect of microglia depletion on AD pathology in these mice, dendritic spine density was measured for the control and depleted groups (FIG. 23D). The ratio of dendritic spine density near to (less than 20 microns), and far away from (more than 50 microns), plaques was measured in the mice from each group. Dendritic spine density near the plaques was significantly increased in the four week and eight week depleted mice relative to the dendritic spine density in the relevant control mice (FIG. 23E). Microglia depletion had no effect on plaque density (FIG. 24), no effect on plaque size (FIG. 25), no effect on astrocytes (FIG. 26), and no effect on activity in an open field task (FIG. 27). Depletion of microglia was confirmed by Iba1 immunohistochemistry (FIG. 28). Without wishing to be bound by theory, this data suggests that dendritic spines near plaques are significantly less stable, and are turned over more rapidly in the presence of microglia.

### Example 6: Microglial Depletion in a Mouse Model of Neuropathic Pain

Spinal microglia are believed to contribute to neuropathic pain. A mouse model is used to determine whether depletion of microglia can prevent or ameliorate neuropathic pain by determining 1) whether spinal microglia are affected similarly to cortical microglia by inhibitor treatment in intact (uninjured) animals, 2) the effects of inhibitor treatment on the focal proliferation of spinal microglia induced by peripheral nerve injury, and 3) how microglial depletion impacts the development of hypersensitivity in a mouse model of neuropathic pain.

### Methods

Spared Nerve Injury (SNI) Model of Neuropathic Pain

The techniques discussed herein are based on Shields et al. (2003, J Pain 4(8): 465-470). The surgeon(s) use aseptic techniques and abide by the IACUC's Rodent Survival Surgery Guidelines. The site of planned incision (over the sciatic nerve at the level of its trifurcation at the popliteal fossa at the back of the knee) is shaved and prepped with betadine followed by alcohol swabbing. Topical lidocaine is applied prior to incision. During and post surgery, the animal is kept warm (e.g., using a circulating heating pad). The procedure is performed as follows: a skin incision is made into the popliteal area on one side, the muscles overlying the sciatic nerve are separated, and the sciatic nerve is isolated. At this level, the sciatic nerve trifurcates into the sural nerve, the common peroneal nerve, and the tibial nerve. The sural and common peroneal nerves are individually tightly ligated with fine silk suture and cut distal to the suture. Care is taken not to contact or damage the spared tibial nerve. In the case of sham surgery, the sciatic nerve is exposed as described, but not manipulated. Subsequently, the muscles are brought back into their original anatomical position, and the overlying skin is closed using surgical staples. This is a unilateral procedure; the contralateral side is left intact. Animals are recovered on a circulating heating pad. They are observed until they have recovered from anesthesia and then returned to the animal room in their cages.

Dosing

For all studies, inhibitor treatment groups receive anti-IL-34 antibody 60 mg/kg i.p. 2×/week+GW2580 150 mg/kg p.o. q.d., and vehicle treatment groups receive the same dose regimen but with vehicle only. The vehicle for anti-IL-34 antibody is sterile phosphate-buffered saline (PBS). The vehicle for GW2580 is MCT. Intraperitoneal (i.p.) and oral (p.o.) dosing is performed in a volume no greater than 10 ml/kg.

Behavioral Testing

Von Frey Test of Mechanical Threshold

Mice are habituated for 45-60 minutes in individual plexiglas test chambers on an elevated wire mesh surface. The chambers are of sufficient size that the animals can move about freely without restraint. Nylon filaments that have been calibrated to deliver precise forces are applied one at a time to the plantar surface of the hindpaw of each animal, following the up-down method (Chaplan et al., 1994, J Neurosci Methods 53:55-63). Briefly, if the mouse withdraws its hindpaw in response to stimulation with a filament, it is stimulated again later with the next weaker filament in the series; if the mouse does not react to a given filament, it is stimulated again with the next stronger filament in the series. Stimulation continues until six responses have been recorded surrounding the withdrawal threshold. Stimuli presented to mice range from 0.008 g to 2.0 g.

Acetone Evaporative Cooling Test

Mice are habituated as described above. A 1 ml syringe without a needle is filled with acetone and held tip upwards, and the plunger is pressed until a small amount of acetone emerges from the tip, held on by surface tension. This bubble of acetone is touched to the plantar surface of one hindpaw of each animal one at a time, and the amount of time the mouse spends reacting to this stimulus is recorded. The acetone begins to evaporate immediately upon contact with the skin, producing a cooling sensation to which the animal typically reacts by shaking the affected hindpaw, holding it aloft, or licking the hindpaw or ankle. Normal mice without neuropathy spend one second or less reacting to the acetone stimulus, while neuropathic mice may spend up to 20 seconds reacting.

Study Groups

### Groups 1-6: Depletion of Microglia Prior to SNI

Experiments using Groups 1-6 (Table 3) are used to determine the maximum effect of microglial depletion on neuropathic pain. Mice in groups 1-6 begin vehicle or inhibitor treatment on Day −7 (seven days prior to SNI). Mice in groups 1-2 are taken down by perfusion under anesthesia after 7 days of vehicle or inhibitor treatment without undergoing SNI surgery, in order to establish a baseline pre-SNI microglial status and to assess the effect of inhibitor treatment on spinal microglia. Mice in groups 3-6 undergo SNI surgery on Day 0. Mice in groups 3-4 are taken down by perfusion under anesthesia on Day 7 after SNI, a time when untreated animals display maximal microglial activation in the spinal cord; this is used to determine how effective the inhibitor treatment is to not only deplete microglia at baseline but also to inhibit microglia proliferation caused by peripheral nerve injury. Mice in groups 5-6 undergo behavioral assessment on Days −1, 1, 3, 7, 10, and 14 relative to SNI. Behavioral assessments consist of the von Frey test of mechanical threshold and the acetone evaporative cooling test (as described previously). After behavioral testing on Day 14 post-SNI, mice in groups 5-6 are euthanized by CO2 inhalation.

TABLE 3
Treatment groups 1-6: Depletion of microglia prior to SNI

| Group | Parameters |
|---|---|
| 1 | Histology - Vehicle treatment, tissue collection prior |
| | to SNI (n = 5) |
| 2 | Histology - Inhibitor treatment, tissue collection prior |
| | to SNI (n = 5) |
| 3 | Histology - Vehicle treatment beginning prior to |
| | SNI, tissue collection 7 days post-SNI (n = 5) |
| 4 | Histology - Inhibitor treatment beginning prior to |
| | SNI, tissue collection 7 days post-SNI (n = 5) |
| 5 | Behavior - Vehicle treatment beginning prior to SNI (n = 10) |
| 6 | Behavior - Inhibitor treatment beginning prior to SNI (n = 10) |

Groups 7-10: Depletion of Microglia after SNI

The ability of inhibitor treatment initiated shortly after a nerve injury to protect against the development of neuropathic pain is evaluated. Experiments using Groups 7-10 (Table 4) are used to determine if dampening microglial response in a clinically relevant setting (i.e. after nerve injury rather than before) is protective against neuropathic pain.

Mice in groups 7-10 undergo SNI surgery on Day 0, and then begin vehicle or inhibitor treatment on Day 3 post-SNI. Mice in groups 7-8 are taken down by perfusion under anesthesia on Day 7 after SNI (when spinal microglial activation are maximal in untreated animals) to assess the effect of inhibitor treatment on microglial activation when administered shortly after nerve injury. Mice in groups 9-10 undergo behavioral assessment on Days −1, 1, 3, 7, 10, and 14 relative to SNI. Behavioral assessments consist of the von Frey test of mechanical threshold and the acetone evaporative cooling test. After behavioral testing on Day 14 post-SNI, mice in groups 9-10 are euthanized by CO2 inhalation.

TABLE 4
Treatment groups 7-10: Depletion of microglia after SNI

| Group | Parameters |
|---|---|
| 7 | Histology - Vehicle treatment beginning 3 days |
| | after SNI, tissue collection 7 days post-SNI (n = 5) |
| 8 | Histology - Inhibitor treatment beginning 3 days |
| | after SNI, tissue collection 7 days post-SNI (n = 5) |
| 9 | Behavior - Vehicle treatment beginning 3 days after SNI |
| | (n = 10) |
| 10 | Behavior - Inhibitor treatment beginning 3 days after SNI |
| | (n = 10) |

### Groups 11-13: Control for Microglia-Independent Effects of the Inhibitor Treatment on Neuropathic Pain

Experiments using Groups 11-13 (Table 5) are used to determine whether the inhibitor treatment is acting through microglia. Administering the treatment at a time when neuropathic pain is still present but microglial involvement is minimal enables the determination of whether the inhibitor is acting to normalize pain thresholds in a manner separate from its effects on microglia.

Mice in groups 11-13 undergo SNI surgery on Day 0. Mice in group 11 are taken down by perfusion under anesthesia on Day 28 after SNI, a time when nerve injury-induced microglial activation returns to baseline. Mice in groups 12-13 undergo behavioral assessment as described above on Days −1, 1, 3, 7, 10, 14, 21, 28, 35, and 42 relative to SNI, and begin vehicle or inhibitor treatment on Day 28 post-SNI. After behavioral testing on Day 42 post-SNI, mice in groups 12-13 are euthanized by CO2 inhalation.

TABLE 5
Treatment groups 11-13: Control for microglia-independent
effects of the inhibitor on neuropathic pain

| Group | Parameters |
|---|---|
| 11 | Histology - Tissue collection 28 days after SNI (n = 5) |
| 12 | Behavior - Vehicle treatment beginning 28 days after SNI |
| | (n = 10) |
| 13 | Behavior - Inhibitor treatment beginning 28 days after SNI |
| | (n = 10) |

### Example 7: Effect of Microglia Depletion on ALS Pathology in the SOD1 Mouse Model

Microglia play numerous roles in both tissue homeostasis and response to damage. Neuroinflammation, including microglia activation, is a hallmark of both familial and sporadic ALS patients, as well as mouse models of the disease.

The effect of microglia depletion on ALS pathology in the SOD1 mouse model is evaluated by assessing microglial activation, astrogliosis, and motor neuron survival after microglia depletion by immunohistochemistry, as well as axon degeneration in the sciatic nerve by EM.

### Methods

Mice are dosed with a neutralizing antibody to IL-34, or a control anti-gp120 antibody IP (<0.25 mL), twice per week for 6 weeks, from 8-14 weeks of age. Mice in groups 2 and 3 are additionally treated with either vehicle (MCT, group 2) or a CSF1R small molecule inhibitor, GW2580 (group 3), PO, once per day at 150 mg/kg (<0.25 mL), for 6 weeks. Details of the experimental design and treatment groups are shown in Table 6. After treatment, ALS pathology is evaluated by assessing microglial activation, astrogliosis, and motor neuron survival, as well as axon degeneration in the sciatic nerve by EM.

TABLE 6
Experimental Design

| Group | Parameters |
|---|---|
| 1 | 10 SOD1 female mice; anti-IL-34 60 mg/kg |
| 2 | 10 SOD1 female mice; anti-gp120 60 mg/kg + MCT |
| 3 | 10 SOD1 female mice; anti-IL-34 60 mg/kg + GW280 |
| | 150 mg/kg |
| 4 | 5 CX3CR1-GFP female mice; anti-IL-34 60 mg/kg |
| 5 | 5 CX3CR1-GFP female mice; no treatment |

1. A method of treating a neurological disease in an individual comprising administering to the individual an effective amount of an anti-IL-34 antibody.

2. A method of treating an individual exhibiting one or more symptoms of a neurological disease comprising administering to the individual an effective amount of an anti-IL-34 antibody.

3. A method of reducing the density of microglia in the brain of an individual comprising administering to the individual an effective amount of an anti-IL-34 antibody.

4. The method of claim 1, wherein the anti-IL-34 antibody is an isolated antibody that binds to human IL-34, which antibody binds to an epitope comprising at least one of amino acid residues Glu103, Leu109, Gln106, Asn150, Leu127, Asn128, Ser184, Leu186, Asn187, Lys44, Glu121, Asp107, Glu111, Ser104, Gln120, Trp116, and Asn61 of a human IL-34, wherein the position of the amino acid residues is based on the position in SEQ ID NO:1, and wherein the antibody inhibits the binding between human IL-34 and human CSF-1R.

5. The method of claim 1, wherein the anti-IL-34 antibody is an isolated antibody that binds to human IL-34, which antibody binds to an epitope comprising at least one of amino acid residues from Glu103 to Asn150 of a human IL-34, wherein the position of the amino acid residues is based on SEQ ID NO:1, and wherein the antibody inhibits the binding between human IL-34 and human CSF-1R.

6-8. (canceled)

9. The method of claim 4, wherein the antibody binds to an epitope comprising at least one of amino acid residues Asn128, Ser184, Leu186, Asn187, Lys44, and Glu121 of the human IL-34, wherein the position of the amino acid residues is based on the position in SEQ ID NO:1.

10. The method of claim 9, wherein the epitope further comprises at least one of amino acid residues Phe40, Asp43, Leu125, Gln189, Thr36, and Val185 of the human IL-34, wherein the position of the amino acid residues is based on the position in SEQ ID NO:1.

11-12. (canceled)

13. The method of claim 4, wherein the antibody binds to an epitope comprising at least one of amino acid residues Asp107, Glu111, Ser104, Gln120, Glu103, Leu109, Trp116, and Asn61 of the human IL-34, wherein the position of the amino acid residues is based on the position in SEQ ID NO:1.

14. The method of claim 13, wherein the epitope further comprises at least one of amino acid residues Pro152, Val108, Leu110, Gln106, Glu123, Leu127, Lys117, Ile60 and Lys55 of the human IL-34, wherein the position of the amino acid residues is based on the position in SEQ ID NO:1.

15-22. (canceled)

23. The method of claim 1 wherein the anti-IL-34 antibody is an isolated antibody that binds to a human IL-34, wherein the antibody inhibits the binding between human IL-34 and human CSF-1R, and wherein the antibody binds to a dimer of the IL-34.

24. The method of claim 23, wherein the antibody comprises (a) a HVR-H3 comprising an amino acid sequence GLGKGSKRGAMDY (SEQ ID NO: 33) or GINQGSKRGAMDY (SEQ ID NO: 32); (b) a HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41); and (c) a HVR-H2 comprising an amino acid sequence RISPYYYYSDYADSVKG (SEQ ID NO: 52) or RISPYSGYTNYADSVKG (SEQ ID NO: 51).

25. The method of claim 23, wherein the antibody comprises (a) a HVR-H1 comprising an amino acid sequence STWIH (SEQ ID NO: 59); (b) a HVR-H2 comprising an amino acid sequence RISPYYYYSDYADSVKG (SEQ ID NO: 52) or RISPYSGYTNYADSVKG (SEQ ID NO: 51); and (c) a HVR-H3 comprising an amino acid sequence GLGKGSKRGAMDY (SEQ ID NO: 33) or GINQGSKRGAMDY (SEQ ID NO: 32).

26. The method of claim 23, wherein the antibody comprises (a) a HVR-L1 comprising an amino acid sequence RASQDVSTAVA (SEQ ID NO: 50); (b) a HVR-L2 comprising an amino acid sequence SASFLYS (SEQ ID NO: 53); and (c) a HVR-L3 comprising an amino acid sequence QQSFYFPNT (SEQ ID NO: 39) or QQSYTTPPT (SEQ ID NO: 43) or QQYTALPYT (SEQ ID NO: 49) or QQYSDLPYT (SEQ ID NO: 45) or QQYSDVPYT (SEQ ID NO: 47) or QQSRTARPT (SEQ ID NO: 41) or QQSFYFPN (SEQ ID NO: 38) or QQSYTTPP (SEQ ID NO: 42) or QQYTALPY (SEQ ID NO: 48) or QQYSDLPY (SEQ ID NO: 44) or QQYSDVPY (SEQ ID NO: 46) or QQSRTARP (SEQ ID NO: 40).

27-41. (canceled)

42. The method of claim 1, wherein the anti-IL-34 antibody is an isolated antibody that binds to human IL-34, wherein the antibody inhibits the binding between human IL-34 and human CSF-1R, and wherein the antibody neutralizes IL-34 activity.

43. The method of claim 42, wherein the antibody comprises (a) a HVR-H3 comprising an amino acid sequence SRGAYRFAY (SEQ ID NO: 56); (b) a HVR-L3 comprising an amino acid sequence QQSYTTPPT (SEQ ID NO: 43); and (c) a HVR-H2 comprising an amino acid sequence SITPASGDTDYADSVKG (SEQ ID NO: 54).

44. The method of claim 42, wherein the antibody comprises (a) a HVR-H1 comprising an amino acid sequence SNYIH (SEQ ID NO: 55); (b) a HVR-H2 comprising an amino acid sequence SITPASGDTDYADSVKG (SEQ ID NO: 54); and (c) a HVR-H3 comprising an amino acid sequence SRGAYRFAY (SEQ ID NO: 56).

45. The method of claim 42, wherein the antibody comprises (a) a HVR-L1 comprising an amino acid sequence RASQDVSTAVA (SEQ ID NO: 50); (b) a HVR-L2 comprising an amino acid sequence SASFLYS (SEQ ID NO: 53); and (c) a HVR-L3 comprising an amino acid sequence QQSYTTPPT (SEQ ID NO: 43).

46-50. (canceled)

51. The method of claim 1, wherein the antibody is a bispecific antibody.

52. The method of claim 51, wherein the bispecific antibody comprises a second binding specificity to human CSF-1.

53. The method of claim 52, wherein the bispecific antibody inhibits binding of human CSF-1 to human CSF-1R.

54-58. (canceled)

59. The method of claim 1 further comprising administering to the individual an effective amount of a CSF-1R inhibitor.

60. The method of claim 59 wherein the CSF-1R inhibitor is a small molecule inhibitor.

61. The method of claim 60 wherein the small molecule inhibitor is GW2580.

62. The method of claim 59 wherein the CSF-1R inhibitor is an anti-CSF-1R antibody.

63. The method of claim 62 wherein the anti-CSF-1R antibody is an isolated antibody that binds human CSF-1R, which antibody binds to an epitope comprising at least one of amino acid residues Arg144, Gln248, Gln249, Ser250, Phe252, and Asn254 of human CSF-1R, wherein the position of amino acid residue is based on the position in SEQ ID NO:2, and wherein the antibody inhibits the binding between human IL-34 and human CSF-1R.

64. The method of claim 63, wherein the antibody binds to an epitope comprising amino acid residue Arg144 of CSF-1R, wherein the position of amino acid residue is based on the position in SEQ ID NO:2.

65. The method of claim 64, wherein the epitope further comprises at least one of amino acid residues Arg142, Arg146, and Arg150 of human CSF-1R, and wherein the position of amino acid residues is based on the position in SEQ ID NO:2.

66-68. (canceled)

69. The method of claim 63, wherein the antibody binds to an epitope comprising at least one of amino acid residues Gln248, Gln249, Ser250, Phe252, and Asn254 of human CSF-1R, wherein the position of amino acid residue is based on the position in SEQ ID NO:2.

70-73. (canceled)

74. The method of claim 1 further comprising administering to the individual an effective amount of an anti-CSF-1 antibody.

75. The method of claim 74 wherein the anti-CSF-1 antibody inhibits binding of human CSF-1 to human CSF-1R.

76. The method of claim 1 wherein the individual is a human.

77. The method of claim 1, wherein the neurological disease is selected from the group consisting of Alzheimer's disease, Parkinson's disease, Huntington's disease, amyotrophic lateral sclerosis, neuropathic pain, prion disease, spinocerebellar ataxia, spinal muscular atrophy, autism, and autism spectrum disorders.

78-83. (canceled)

84. A kit comprising a pharmaceutical composition comprising an anti-IL-34 antibody and a pharmaceutically acceptable carrier.

85-90. (canceled)
